(19) United States
(12) Patent Application Publication
(10) Pub. No.: US 2023/0037448 A1
(43) Pub. Date: Feb. 9, 2023

(54) FLUORINATED QUINOLINE AND QUINOXALINE DERIVATIVES AS DIHYDROOROTATE DEHYDROGENASE (DHODH) INHIBITORS FOR THE TREATMENT OF CANCER, AUTOIMMUNE AND INFLAMMATORY DISEASES

(71) Applicant: Janssen Biotech, Inc., Horsham, PA (US)

(72) Inventors: Justin Cisar, North Wales, PA (US); Colleen Keohane, Conshohocken, PA (US); Scott Kuduk, Harleysville, PA (US)

(21) Appl. No.: 17/773,098

(22) PCT Filed: Oct. 30, 2020
(86) PCT No.: PCT/IB2020/060221

§ 371 Date: Apr. 29, 2022

Related U.S. Application Data
(60) Provisional application No. 62/929,163, filed on Nov. 1, 2019.

(51) Int. Cl. C07D 401/04; C07D 401/14; C07D 405/14; A61P 35/00; A61P 35/02

(52) U.S. Cl. CPC C07D 401/04; C07D 401/14; C07D 405/14; A61P 35/00; A61P 35/02

(57) ABSTRACT

Disclosed are compounds, compositions and methods for treating diseases, disorders, or medical conditions that are affected by the modulation of DHODH. Such compounds are represented by Formula (I) as follows:

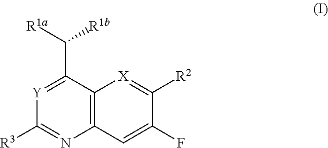

wherein R^{1a}, R^{1b}, R², and R³, are defined herein.

### CROSS-REFERENCE TO RELATED APPLICATIONS

This application claims the benefit of priority of U.S. Provisional Application No. 62/929,163, filed on Nov. 1, 2019, which is incorporated by reference herein, in its entirety and for all purposes.

### FIELD OF THE INVENTION

The present invention relates to novel compounds that are dihydroorotate dehydrogenase (DHODH) inhibitors. These compounds may be useful for the treatment of a disease, disorder, or medical condition where there is an advantage in inhibiting DHODH. The invention also relates to pharmaceutical compositions comprising one or more of such compounds, to processes to prepare such compounds and compositions, and to the use of such compounds or pharmaceutical compositions for the method of treatment of cancer, and autoimmune and inflammatory diseases, syndromes, and disorders.

### BACKGROUND OF THE INVENTION

Acute myelogenous leukemia (AML) is a clonal disease of the blood and bone marrow resulting from mutations that occur in normal hematopoietic stem cells. AML is a heterogenous disease in that it presents with a range of cytogenetic, morphological and immunophenotypic features, and is characterized by an accumulation of clonal, abnormal myeloid progenitor cells, known as myeloblasts. These cells demonstrate disruption of normal myeloid differentiation and excessive proliferation, resulting in the decreased formation of hematopoietic cells. Disease remission can be achieved with standard induction chemotherapy, but refractory and relapsed disease remains a challenge due to persistence of leukemic stem cells. Therefore, AML represents an unmet medical need with >20,000 new cases per year in the US with 5-year overall survival below 30% (Stein E T et al., Health Qual Life Outcomes 16: 193, 2018).

Differentiation therapy is considered an attractive approach to AML treatment based on the knowledge that differentiation and loss of stem cell self-renewal are coupled in normal cells. Treatment of acute promyelocytic leukemia, which represents 10-15% of all AML, with all-trans retinoic acid is the paradigm for differentiation therapy. Retinoic acid targets the promyelocytic leukemia protein (PML)-retinoic acid receptor-α (RAR-α) fusion protein encoded by a t(15,17) chromosomal translocation. Targeting PML-RAR specifically lifts the transcriptionally mediated differentiation block induced by the fusion protein and early clinical trials with single agent ATRA demonstrated complete hematologic remission in all treated patients (McCulloch D et al. Onco Targets Ther 2017; 10: 1585-1601; Nowak D et al. Blood 113: 3655, 2009).

Although differentiation therapy is successful, it is only applicable to a small population of AML patients. Research efforts have aimed at identifying additional differentiation inducing agents, but with limited success. Recently dihydroorotate dehydrogenase (DHODH) emerged as a potentially more broadly applicable differentiation target in a phenotypic screen aimed at identifying small molecules that overcome blockade of the maturation of primary murine bone marrow cells expressing the homeobox protein HoxA9. Ibis protein is a key transcription factor involved in balancing stem cell maintenance/differentiation and is normally expressed in hematopoietic progenitor cells and downregulated upon induction of differentiation and has been found to be widely overexpressed in AML (Sykes et al., Cell 167: 171, 2016).

DHODH is a flavin mononucleotide (FMN) flavoprotein located in the inner mitochondrial membrane that catalyzes the oxidation of dihydroorotate to orotate, the fourth step in the de novo pyrimidine biosynthesis pathway. Inhibition of DHODH leads to decreased pyrimidine synthesis important precursors for nucleotide synthesis, but also glycoprotein and phospholipid biosynthesis (Reis RAG et al., Archives Biochem Biophysics 632: 175, 2017; Vyas V K et al., Mini Rev Med Chem 11: 1039, 2011).

DHODH is a validated target for the treatment of autoimmune diseases with the FDA approved small molecule DHODH inhibitors leflunomide and teriflunomide for rheumatoid arthritis and multiple sclerosis, respectively (Lolli M L et al., Recent patents on Anti-Cancer Drug Discovery 13: 86, 2018).

Since the first observation by Sykes et al. demonstrating that DHODH inhibition drives AML differentiation in vitro, as evidenced by upregulation of the differentiation markers CD11b and CD14, and results in dose dependent anti-leukemic effects, decreased leukemic stem cells and prolonged survival in vivo, additional evidence emerged demonstrating that small molecule DHODH inhibitors mediate antiproliferative activity against AML cells with concomitant cell cycle arrest, upregulation of CD11b and CD14, and induction of apoptosis (Wu D et al., Haematologica 103: 1472, 2018; Sainas S et al., J Med Chem 61: 6034, 2018; Cao L et al., Mol Cancer Ther, October 23rd Epub ahead of print). Moreover, preclinical solid tumor in vitro and in vivo models demonstrated effectiveness of DHODH inhibition and DHODH was identified as a synthetic lethality in PTEN and KRAS mutant solid tumors (Pharmacology and Therapeutics, Epub Oct. 19, 2018; Mathur D et al., Cancer Discovery 7: 1, 2017; Cell Chemical Biology 25: 1, 2018).

Thus, there remains a need for DHODH inhibitors that provide a therapeutic benefit to patients suffering from cancer and/or inflammatory and immunological diseases.

### SUMMARY OF THE INVENTION

Embodiments of the present invention relate to compounds, pharmaceutical compositions containing them, methods of making and purifying them, methods of using them as inhibitors of DHODH enzymatic activity and methods for using them in the treatment of a subject suffering from or diagnosed with a disease, disorder, or medical condition such as autoimmune or inflammatory disorders, or diseases such as cancer.

Embodiments of this invention are compounds of Formula (I),

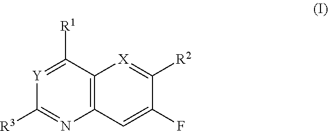

wherein
X is CH or N;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
   R² is selected from the group consisting of:

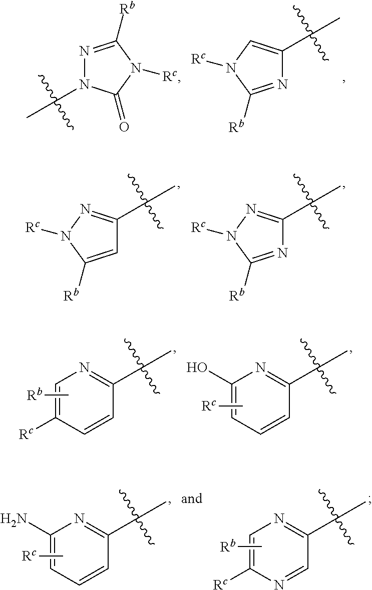

where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₃₋₆cycloalkyl, and tetrahydro-2H-pyranyl;
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

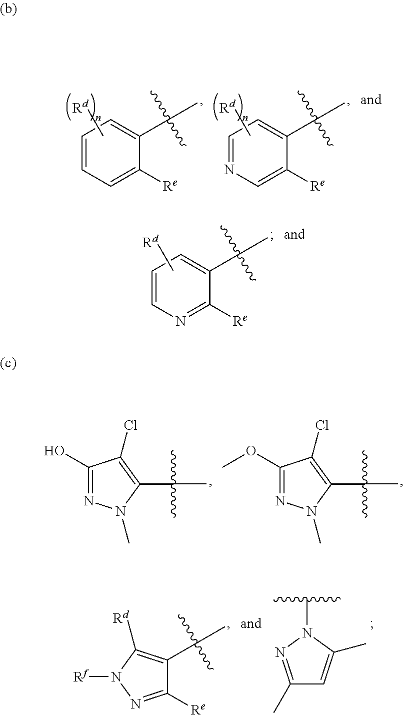

where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, isotope, N-oxide, solvate, or stereoisomer thereof.

The present invention further provides methods for treating or ameliorating a disease, syndrome, condition, or disorder in a subject, including a mammal and/or human in which the disease, syndrome, condition, or disorder is affected by the inhibition of DHODH enzymatic activity, including but not limited to, cancer and/or inflammatory or immunological diseases, using a compound of Formula (I) or a pharmaceutically acceptable salt, isotope, N-oxide, solvate, or stereoisomer thereof.

Additional embodiments, features, and advantages of the invention will be apparent from the following detailed description and through practice of the invention.

### DETAILED DESCRIPTION OF THE INVENTION

Unless defined otherwise, all technical and scientific terms used herein have the same meaning as is commonly understood by one of ordinary skill in art. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

With reference to substituents, the term “independently” refers to the situation where when more than one substituent is possible, the substituents may be the same or different from each other.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Unless qualified specifically in particular instances of use, the term “alkyl” refers to a straight- or branched-chain alkyl group having from 1 to 8 carbon atoms in the chain.

Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. “C₁₋₆alkyl” refers to straight- or branched-chain alkyl group having from 1 to 6 carbon atoms in the chain. “C₁₋₄alkyl” refers to straight- or branched-chain alkyl group having from 1 to 4 carbon atoms in the chain.

The term “cycloalkyl” refers to a saturated or partially saturated, monocyclic, fused polycyclic, or spiro polycyclic carbocycle having from 3 to 12 ring atoms per carbocycle. “C₃₋₆cycloalkyl” refers to a carbocycle having from 3 to 6 ring atoms per carbocycle. Illustrative examples of cycloalkyl groups include the following entities, in the form of properly bonded moieties:

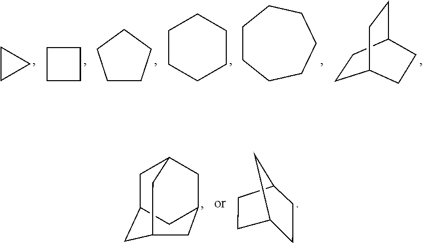

The term “halogen” or “halo” represents chlorine, fluorine, bromine, or iodine.

The term “haloalkyl” refers to a straight- or branched-chain alkyl group having from 1 to 6 carbon atoms in the chain optionally substituting hydrogens with halogens. The term “C₁₋₆ haloalkyl” as used here refers to a straight- or branched-chain alkyl group having from 1 to 6 carbon atoms in the chain, optionally substituting hydrogens with halogens. The term “C₁₋₄ haloalkyl” as used here refers to a straight- or branched-chain alkyl group having from 1 to 4 carbon atoms in the chain, optionally substituting hydrogens with halogens. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “aryl” refers to a monocyclic, aromatic carbocycle (ring structure having ring atoms that are all carbon) having 6 atoms per ring. (Carbon atoms in the aryl groups are sp2 hybridized.)

The term “phenyl” represents the following moiety:

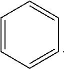

The term “oxetanyl” represents the following moiety:

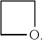

The term “tetrahydrofuranyl” represents the following moiety:

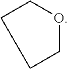

The term “tetrahydropyranyl” represents the following moiety:

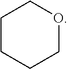

The term “piperidinyl” represents the following moiety:

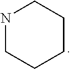

The term “heteroaryl” refers to a monocyclic or fused bicyclic heterocycle (ring structure having ring atoms selected from carbon atoms and up to four heteroatoms selected from nitrogen, oxygen, and sulfur) having from 3 to 9 ring atoms per heterocycle. Illustrative examples of heteroaryl groups include the following entities, in the form of properly bonded moieties:

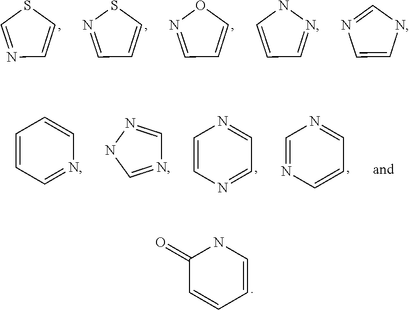

The term “tautomeric” or “tautomeric form” refers to structural isomers of different energies that are interconvertible through low energy barriers. For example, proton tautomers (also known as proton tautomers) include interconversions through the transfer of protons, such as keto-enol and imine-enamine isomerization. The valence tautomers include interconversions by restructuring some bond electrons.

For example, hydroxypyridine or the tautomeric pyridone is represented below.

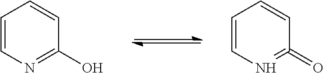

For example, pyrazole tautomers are represented below.

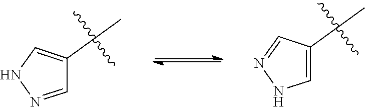 [SMILES: *c1cn[nH]c1 ; *c1cn[nH]c1]

Those skilled in the art will recognize that the species of heterocycloalkyl, cycloalkyl, heteroaryl and aryl groups listed or illustrated above are not exhaustive, and that additional species within the scope of these defined terms may also be selected.

The term “variable point of attachment” means that a group is allowed to be attached at more than one alternative position in a structure. The attachment will always replace a hydrogen atom on one of the ring atoms. In other words, all permutations of bonding are represented by the single diagram, as shown in the illustrations below.

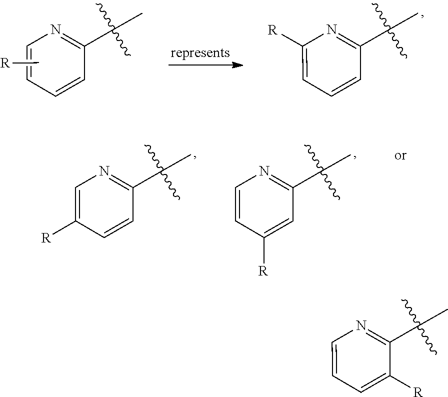

Those skilled in the art will recognize that that if more than one such substituent is present for a given ring, the bonding of each substituent is independent of all of the others. The groups listed or illustrated above are not exhaustive.

As used herein, the term “or” means “and/or” unless stated otherwise.

As used herein, the terms “including”, “containing” and “comprising” are used in their open, non-limiting sense.

As used herein, the term “composition” is intended to encompass a product comprising the specified ingredients in the specified amounts, as well as any product which results, directly or indirectly, from combination of the specified ingredients in the specified amounts.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physiological or biochemical parameter associated with or causative of the disease, condition, syndrome or disorder, including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

The terms “subject” and “patient” are used interchangeably herein and may refer to an animal, preferably a mammal, most preferably a human.

As used herein, the terms active compound, pharmaceutical agent and active ingredient are used interchangeably to refer to a pharmaceutically active compound. Other ingredients in a drug composition, such as carriers, diluents or excipients, may be substantially or completely pharmaceutically inert. A pharmaceutical composition (also referred to herein as a composition or formulation) may comprise the active ingredient in combination with one or more carriers and/or one or more excipients and/or one or more diluents.

The term “therapeutically effective amount” (used interchangeably herein with “effective amount”) refers to an amount (e.g., of an active compound or pharmaceutical agent, such as a compound of the present invention), which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease. Stated another way, the term therapeutically effective amount may refer to an amount that, when administered to a particular subject, achieves a therapeutic effect by inhibiting, alleviating or curing a disease, condition, syndrome or disorder in the subject or by prophylactically inhibiting, preventing or delaying the onset of a disease, condition, syndrome or disorder, or symptom(s) thereof. A therapeutically effective amount may be an amount which relieves to some extent one or more symptoms of a disease, condition, syndrome or disorder in a subject; and/or returns to normal either partially or completely one or more physiological or biochemical parameters associated with or causative of the disease, condition, syndrome or disorder; and/or reduces the likelihood of the onset of the disease, condition, syndrome or disorder, or symptom(s) thereof.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

A “pharmaceutically acceptable salt” is intended to mean a salt of an acid or base of a compound represented by Formula (I) (as well as compounds of Formula (IA), (IB), and (IC)) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response.

Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

Compounds of Formula (I) may contain at least one nitrogen of basic character, so desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, funaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents.

Compounds of Formula (I) may contain a carboxylic acid moiety, a desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, piperazine, N-methyl-glucamine and tromethamine and inorganic salts derived from sodium, calcium, potassium, magnesium, manganese, iron, copper, zinc, aluminum, and lithium.

Each compound used herein may be discussed interchangeably with respect to its chemical formula, chemical name, abbreviation, etc.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof. Additionally, any formula given herein is intended to refer also to any one of: hydrates, solvates, polymorphs and of such compounds, and mixtures thereof, even if such forms are not listed explicitly.

The term “R” at a stereocenter designates that the stereocenter is purely of the R-configuration as defined in the art; likewise, the term “S” means that the stereocenter is purely of the S-configuration. As used herein, the term “RS” refers to a stereocenter that exists as a mixture of the R- and S-configurations.

Compounds containing one stereocenter drawn without a stereo bond designation are a mixture of 2 enantiomers. Compounds containing 2 stereocenters both drawn without stereo bond designations are a mixture of 4 diastereomers. Compounds with 2 stereocenters both labeled “RS” and drawn with stereo bond designations are a 2-component mixture with relative stereochemistry as drawn. Unlabeled stereocenters drawn without stereo bond designations are a mixture of the R- and S-configurations. For unlabeled stereocenters drawn with stereo bond designations, the absolute stereochemistry is as depicted.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Reference to a compound herein stands for a reference to any one of: (a) the recited form of such compound, and (b) any of the forms of such compound in the medium in which the compound is being considered when named. For example, reference herein to a compound such as R—COOH, encompasses reference to any one of: for example, R—COOH(s), R—COOH(sol), and R—COO-(sol). In this example, R—COOH(s) refers to the solid compound, as it could be for example in a tablet or some other solid pharmaceutical composition or preparation; R—COOH(sol) refers to the undissociated form of the compound in a solvent; and R—COO-(sol) refers to the dissociated form of the compound in a solvent, such as the dissociated form of the compound in an aqueous environment, whether such dissociated form derives from R—COOH, from a salt thereof, or from any other entity that yields R—COO— upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH(aq) and/or R—COO-(aq), where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds of the invention in a form that exceeds natural abundances include isotopes of hydrogen, carbon, nitrogen, oxygen, phosphorous, fluorine, chlorine, and iodine, such as ²H (or chemical symbol D), ³H (or chemical symbol T), ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H, or D) may afford certain therapeutic advantages resulting from greater metabolic stability, for example increased in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

The term Cₙ₋ₘ alkyl refers to an aliphatic chain, whether straight or branched, with a total number N of carbon members in the chain that satisfies n≤N≤m, with m>n.

When the same plurality of substituents is assigned to various groups, the specific individual substituent assignment to each of such groups is meant to be independently made with respect to the specific individual substituent assignments to the remaining groups. By way of illustration, but not as a limitation, if each of groups Q and R can be H or F, the choice of H or F for Q is made independently of the choice of H or F for R, so the choice of assignment for Q does not determine or condition the choice of assignment for R, or vice-versa, unless it is expressly indicated otherwise. Illustrative claim recitation in this regard would read as “each of Q and R is independently H or F”, or “each of Q and R is independently selected from the group consisting of H and F”.

In another example, a zwitterionic compound would be encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts, and dipolar ions in the known and well-established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention.

No further examples in this regard are provided herein because the interactions and transformations in a given medium that lead to the various forms of a given compound are known by any one of ordinary skill in the art.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein.

The nomenclature “Cᵢ-Cⱼ” with j>i, when applied herein to a class of substituents, is meant to refer to embodiments of this invention for which each and every one of the number of carbon members, from i to j including i and j, is independently realized. By way of example, the term C₁-C₃ refers independently to embodiments that have one carbon member (C₁), embodiments that have two carbon members (C₂), and embodiments that have three carbon members (C₃).

Embodiments of this invention include compounds of Formula (I),

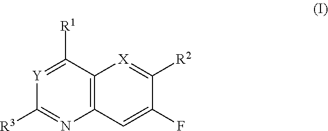

wherein
X is CH or N;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of:

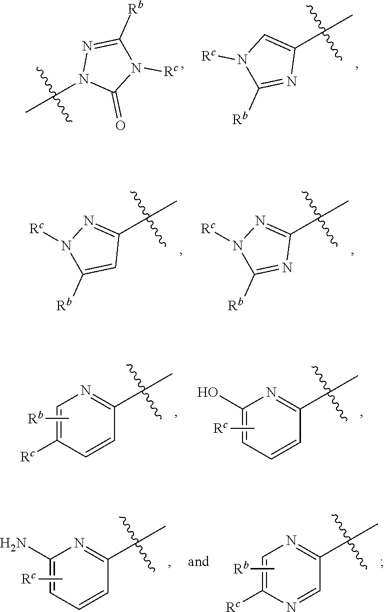

where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₃₋₆cycloalkyl, and tetrahydro-2H-pyranyl;
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

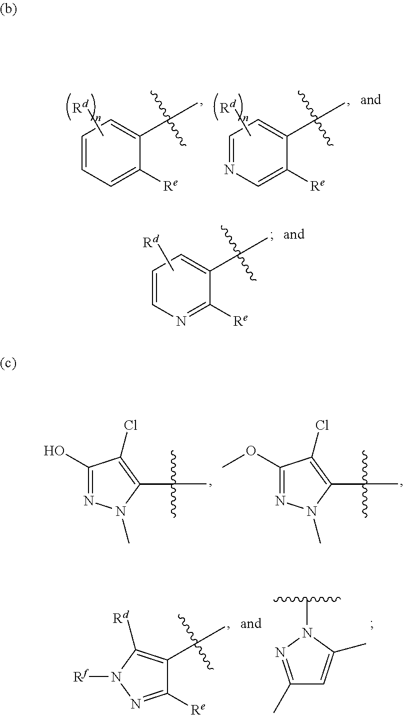

where
   R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
   R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
   R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
   n is 1, or 2;
      or pharmaceutically acceptable salts, isotopes, tautomers, N-oxides, solvates, or stereoisomers thereof.

An additional embodiment of the invention is a compound of Formula (I) wherein

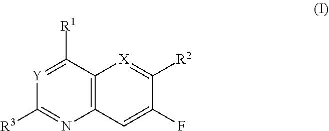

wherein
X is CH;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of:

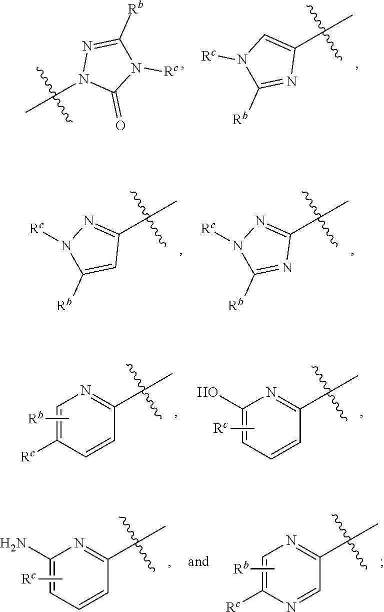

where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₃₋₆cycloalkyl, and tetrahydro-2H-pyranyl;
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

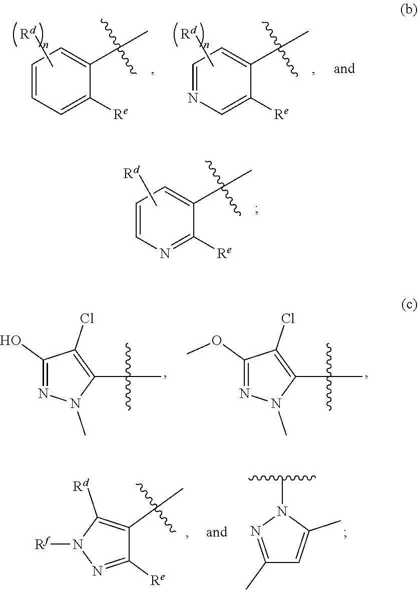

where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2.

An additional embodiment of the invention is a compound of Formula (I) wherein X is CH, and Y is CH.

An additional embodiment of the invention is a compound of Formula (I) wherein X is CH, and Y is N.

An additional embodiment of the invention is a compound of Formula (I) wherein R¹ is C₁₋₆alkyl; C₁₋₆alkyl substituted with OH, or OCH₃; C₂₋₆alkenyl; C₁₋₄haloalkyl; C₁₋₄haloalkyl substituted with OH, or OCH₃; C₃₋₆cycloalkyl; C₁₋₆cycloalkyl independently substituted with one, two, three or four members each independently selected from the group consisting of: halo, OH, C₁₋₆alkyl, and C₁₋₄haloalkyl; oxetanyl; tetrahydrofuranyl; and tetrahydropyranyl.

An additional embodiment of the invention is a compound of Formula (I) wherein R¹ is CH(CH₃)₂.

An additional embodiment of the invention is a compound of Formula (I) wherein R¹ is CH(CH₃)(CF₃).

An additional embodiment of the invention is a compound of Formula (I) wherein R¹ is

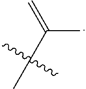 [SMILES: *C(=C)C]

An additional embodiment of the invention is a compound of Formula (I) wherein R¹ is cyclopropyl, cyclobutyl, cyclopentyl, or cyclohexyl; cyclopropyl, cyclobutyl, cyclopentyl, or cyclohexyl each independently substituted with one, two, three or four members selected from the group consisting of: halo, OH, C₁₋₄alkyl, and C₁₋₄haloalkyl; oxetanyl; tetrahydrofuranyl; and tetrahydropyranyl.

An additional embodiment of the invention is a compound of Formula (I) wherein R² is

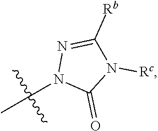

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl.

An additional embodiment of the invention is a compound of Formula (I) wherein
R² is

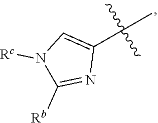

where R^{b} is C₁₋₄alkyl substituted with OH; and R^{c} is C₁₋₄alkyl.

An additional embodiment of the invention is a compound of Formula (I) wherein R² is

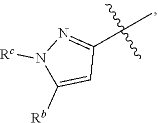

where R^{b} is C₁₋₄alkyl substituted with OH, halo, CN, OC₁₋₄alkyl, OC₁₋₄haloalkyl or OC₃₋₆cycloalkyl; and R^{c} is C₁₋₄alkyl, C₁₋₄haloalkyl, or C₃₋₆cycloalkyl.

An additional embodiment of the invention is a compound of Formula (I) wherein R² is

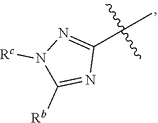

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl.

An additional embodiment of the invention is a compound of Formula (I) wherein R² is

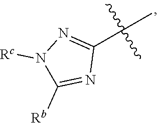

where R^{b} is C₁₋₄alkyl substituted with OH; and R^{c} is C₁₋₄alkyl.

An additional embodiment of the invention is a compound of Formula (I) wherein R² is

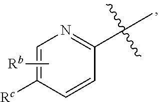

where
R^{b} is C₁₋₄alkyl substituted with OH, halo, CN, OC₁₋₄alkyl, OC₁₋₄haloalkyl or OC₃₋₆cycloalkyl; and R^{c} is C₁₋₄alkyl, C₁₋₄haloalkyl, or C₃₋₆cycloalkyl.

An additional embodiment of the invention is a compound of Formula (I) wherein R² is

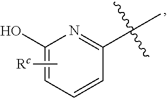

where R^{c} is C₁₋₄alkyl, C₁₋₄haloalkyl, or C₃₋₆cycloalkyl.

An additional embodiment of the invention is a compound of Formula (I) wherein R² is

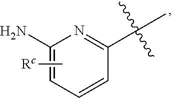

where R^{c} is C₁₋₄alkyl, C₁₋₄haloalkyl, or C₃₋₆cycloalkyl.

An additional embodiment of the invention is a compound of Formula (I) wherein R² is

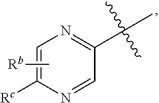

where

R^{b} is C₁₋₄alkyl substituted with OH, halo, CN, OC₁₋₄alkyl, OC₁₋₄haloalkyl or OC₃₋₆cycloalkyl; and

R^{c} is C₁₋₄alkyl, C₁₋₄haloalkyl, or C₃₋₆cycloalkyl.

An additional embodiment of the invention is a compound of Formula (I) wherein R² is

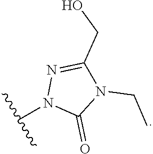 [SMILES: *n1nc(CO)n(CC)c1=O]

An additional embodiment of the invention is a compound of Formula (I) wherein R³ is O—(C₁₋₄alkyl), N(C₁₋₄alkyl)₂, piperidinyl, piperidinyl substituted with CH₃, O—C₃₋₆cycloalkyl, or N—C₁₋₆cycloalkyl.

An additional embodiment of the invention is a compound of Formula (I) wherein R³ is

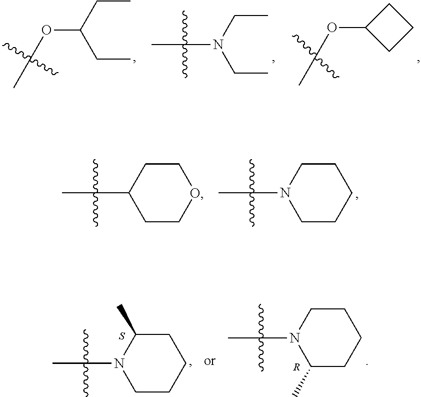

An additional embodiment of the invention is a compound of Formula (I) wherein R³ is

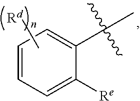

where
R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; and OC₁₋₄alkyl;
R^{c} is halo; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
n is 1 or 2.

An additional embodiment of the invention is a compound of Formula (I) wherein R³ is

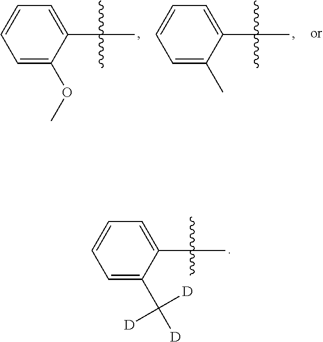

An additional embodiment of the invention is a compound of Formula (I) wherein R³ is

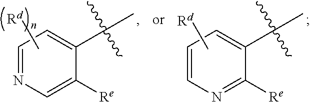

wherein R^{d} is independently selected from the group consisting of: CH₃, OCH₃ and OH; R is halo, CH₃, or OCH₃; and n is 1 or 2.

An additional embodiment of the invention is a compound of Formula (I) wherein R³ is

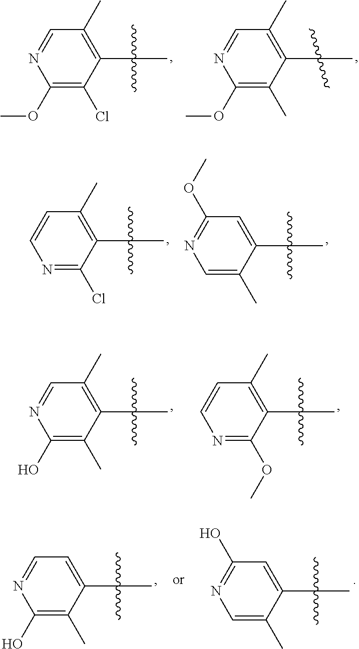

An additional embodiment of the invention is a compound of Formula (I) wherein R³ is selected from the group consisting of:

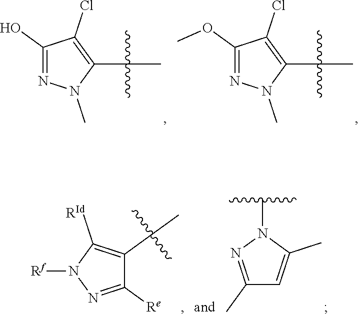

where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl;
R^{e} is halo; C₁₋₄alkyl; C₁₋₄alkyl substituted with OH, OCH₃, SCH₃, or OCF₃; C₁₋₄haloalkyl; C₃₋₆cycloalkyl; or C₁₋₄haloalkyl substituted with OH, or OCH₃; and
R^{f} is H; C₁₋₆alkyl; C₁₋₆alkyl substituted with OH, OCH₃, SCH₃, or OCF₃; C₁₋₄haloalkyl; or C₁₋₄haloalkyl substituted with OH, or OCH₃.

An additional embodiment of the invention is a compound of Formula (I) wherein R³ is

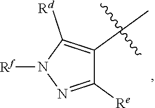

wherein
R^{d} is H, Cl, C₁₋₄alkyl or C₁₋₄haloalkyl;
R^{e} is halo, C₁₋₄alkyl, C₁₋₄haloalkyl or cyclopropyl; and
R_{f} is H.

An additional embodiment of the invention is a compound of Formula (I) wherein R³ is

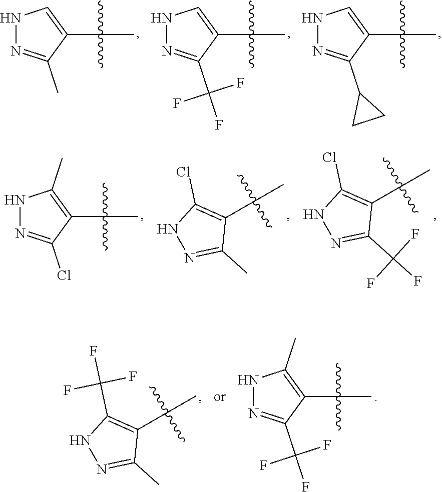

An additional embodiment of the invention is a compound of Formula (I) wherein R³ is

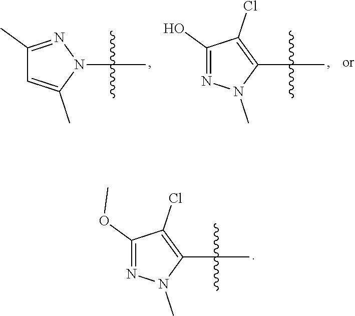

An additional embodiment of the current invention is a compound selected from the compounds shown below in Table 1, and pharmaceutically acceptable salts, isotopes, N-oxides, solvates, and stereoisomers thereof:

TABLE 1

| Ex # | Compound Name |
|---|---|
| 1 | 4-Ethyl-1-(7-fluoro-4-isopropyl-2-(2-methoxyphenyl)quinolin-6-yl)-3- |
| | (hydroxymethyl)-1H-1,2,4-triazol-5(4H)-one; |
| 2 | 1-(2-(3-Chloro-5-methyl-1H-pyrazol-4-yl)-7-fluoro-4-isopropylquinolin-6- |
| | yl)-4-ethyl-3-(hydroxymethyl)-1H-1,2,4-triazol-5(4H)-one; |
| 3 | 4-Ethyl-1-(7-fluoro-4-isopropyl-2-(5-methyl-3-(trifluoromethyl)-1H- |
| | pyrazol-4-yl) quinolin-6-yl)-3-(hydroxymethyl)-1H-1,2,4-triazol-5(4H)- |
| | one; |
| 4 | 4-Ethyl-1-(7-fluoro-4-isopropyl-2-(2-methoxy-3,5-dimethylpyridin-4- |
| | yl)quinolin-6-yl)-3-(hydroxymethyl)-1H-1,2,4-triazol-5(4H)-one; |
| 5 | 4-Ethyl-1-(7-fluoro-4-isopropyl-2-(pentan-3-yloxy)quinolin-6-yl)-3- |
| | (hy droxymethyl)-1H-1,2,4-triazol-5(4H)-one; |
| 6 | 1-(2-Cyclobutoxy-7-fluoro-4-isopropylquinolin-6-yl)-4-ethyl-3- |
| | (hy droxymethyl)-1H-1,2,4-triazol-5(4H)-one; |
| 7 | 1-(2-(3-Chloro-2-methoxy-5-methylpyridin-4-yl)-7-fluoro-4- |
| | isopropylquinolin-6-yl)-4-ethyl-3-(hydroxymethyl)-1H-1,2,4-triazol-5(4H)- |
| | one; |
| 8 | 4-Ethyl-1-(7-fluoro-4-isopropyl-2-(2-methoxy-5-methylpyridin-4- |
| | yl)quinolin-6-yl)-3-(hydroxymethyl)-1H-1,2,4-triazol-5(4H)-one; |
| 9 | 4-Ethyl-1-(7-fluoro-4-isopropyl-2-(o-tolyl)quinolin-6-yl)-3- |
| | (hydroxymethyl)-1H-1,2,4-triazol-5(4H)-one; |
| 10 | 4-Ethyl-2-(7-fluoro-4-isopropyl-2-(o-tolyl)quinazolin-6-yl)-5- |
| | (hydroxymethyl)-2,4-dihydro-3H-1,2,4-triazol-3-one; |
| 11 | 4-Ethyl-2-(7-fluoro-4-isopropyl-2-(2-methoxy-4-methylpyridin-3- |
| | yl)quinolin-6-yl)-5-(hydroxymethyl)-2,4-dihydro-3H-1,2,4-triazol-3-one; |
| 12 | 1-(2-(3,5-Dimethyl-1H-pyrazol-1-yl)-7-fluoro-4-isopropylquinolin-6-yl)- |
| | 4-ethyl-3-(hydroxymethyl)-1H-1,2,4-triazol-5(4H)-one; |
| 13 | 1-(2-(Diethylamino)-7-fluoro-4-isopropylquinolin-6-yl)-4-ethyl-3- |
| | (hydroxymethyl)-1H-1,2,4-triazol-5(4H)-one; |
| 14 | 4-Ethyl-1-(7-fluoro-4-isopropyl-2-(piperidin-1-yl)quinolin-6-yl)-3- |
| | (hydroxymethyl)-1H-1,2,4-triazol-5(4H)-one; |
| 15 | 1-(2-(3-Cyclopropyl-1H-pyrazol-4-yl)-7-fluoro-4-isopropylquinolin-6-yl)- |
| | 4-ethyl-3-(hydroxymethyl)-1H-1,2,4-triazol-5(4H)-one; |
| 16 | 4-Ethyl-2-(7-fluoro-4-isopropyl-2-(3-methyl-1H-pyrazol-4-yl)quinolin-6- |
| | yl)-5-(hydroxymethyl)-2,4-dihydro-3H-1,2,4-triazol-3-one; |
| 17 | (S)-4-Ethyl-2-(7-fluoro-4-isopropyl-2-(2-methylpiperidin-1-yl)quinolin-6- |
| | yl)-5-(hydroxymethyl)-2,4-dihydro-3H-1,2,4-triazol-3-one; |
| 18 | (R)-4-Ethyl-2-(7-fluoro-4-isopropyl-2-(2-methylpiperidin-1-yl)quinolin-6- |
| | yl)-5-(hydroxymethyl)-2,4-dihydro-3H-1,2,4-triazol-3-one; |
| 19 | 4-Ethyl-2-(7-fluoro-2-(2-hydroxy-3,5-dimethylpyridin-4-yl)-4- |
| | isopropylquinolin-6-yl)-5-(hydroxymethyl)-2,4-dihydro-3H-1,2,4-triazol-3- |
| | one; |
| 20 | 4-Ethyl-1-(7-fluoro-4-isopropyl-2-(tetrahydro-2H-pyran-4-yl)quinolin-6- |
| | yl)-3-(hydroxymethyl)-1H-1,2,4-triazol-5(4H)-one; |
| 21 | 6-(4-Ethyl-3-(hydroxymethyl)-5-oxo-4,5-dihydro-1H-1,2,4-triazol-1-yl)-7- |
| | fluoro-4-isopropyl-2-(o-tolyl)quinoline 1-oxide; |
| 22 | 4-Ethyl-1-(7-fluoro-2-(2-hydroxy-3-methylpyridin-4-yl)-4- |
| | isopropylquinolin-6-yl)-3-(hydroxymethyl)-1H-1,2,4-triazol-5(4H)-one; |
| 23 | 4-Ethyl-1-(7-fluoro-4-isopropyl-2-(3-(trifluoromethyl)-1H-pyrazol-4- |
| | yl)quinolin-6-yl)-3-(hydroxymethyl)-1H-1,2,4-triazol-5(4H)-one; |
| 24 | 4-Ethyl-1-(7-fluoro-2-(2-hydroxy-5-methylpyridin-4-yl)-4- |
| | isopropylquinolin-6-yl)-3-(hydroxymethyl)-1H-1,2,4-triazol-5(4H)-one; |
| 25 | 2-(2-(4-Chloro-3-hydroxy-1-methyl-1H-pyrazol-5-yl)-7-fluoro-4- |
| | isopropylquinolin-6-yl)-4-ethyl-5-(hydroxymethyl)-2,4-dihydro-3H-1,2,4- |
| | triazol-3-one; |
| 26 | 2-(2-(4-Chloro-3-methoxy-1-methyl-1H-pyrazol-5-yl)-7-fluoro-4- |
| | isopropylquinolin-6-yl)-4-ethyl-5-(hydroxymethyl)-2,4-dihydro-3H-1,2,4- |
| | triazol-3-one; |
| 27 | 1-(2-(5-Chloro-3-(trifluoromethyl)-1H-pyrazol-4-yl)-7-fluoro-4- |
| | isopropylquinolin-6-yl)-4-ethyl-3-(hydroxymethyl)-1H-1,2,4-triazol-5(4H)- |
| | one; |
| 28 | (3-(7-Fluoro-4-isopropyl-2-(3-methyl-5-(trifluoromethyl)-1H-pyrazol-4- |
| | yl)quinolin-6-y 1)-1-methyl-1H-1,2,4-triazol-5-yl)methanol; |
| 29 | 1-(3-(7-Fluoro-4-isopropyl-2-(3-methyl-5-(trifluoromethyl)-1H-pyrazol-4- |
| | yl)quinolin-6-yl)-1-methyl-1H-1,2,4-triazol-5-yl)ethan-1-ol; |
| 30 | (3-(2-(5-Chloro-3-methyl-1H-pyrazol-4-yl)-7-fluoro-4-isopropylquinolin- |
| | 6-yl)-1-methyl-1H-1,2,4-triazol-5-yl)methanol; |
| 31 | 1-(3-(2-(5-Chloro-3-methyl-1H-pyrazol-4-yl)-7-fluoro-4- |
| | isopropylquinolin-6-yl)-1-methyl-1H-1,2,4-triazol-5-yl)ethan-1-ol; |
| 32 | (4-(2-(5-Chloro-3-methyl-1H-pyrazol-4-yl)-7-fluoro-4-isopropylquinolin- |
| | 6-yl)-1-methyl-1H-imidazol-2-yl)methanol; |
| 33 | (4-(7-Fluoro-4-isopropyl-2-(3-methyl-5-(trifluoromethyl)-1H-pyrazol-4- |
| | yl)quinolin-6-yl)-1-methyl-1H-imidazol-2-yl)methanol; |
| 34 | 4-Ethyl-2-(7-fluoro-4-isopropyl-2-(3-methyl-5-(trifluoromethyl)-1H- |
| | pyrazol-4-yl)quinazolin-6-yl)-5-(hydroxymethyl)-2,4-dihydro-3H-1,2,4- |
| | triazol-3-one; |
| 35 | 1-(2-(2-Chloro-4-methylpyridin-3-yl)-7-fluoro-4-isopropylquinolin-6-yl)- |
| | 4-ethyl-3-(hydroxymethyl)-1H-1,2,4-triazol-5(4H)-one; |
| 36 | (S*)-4-Ethyl-1-(7-fluoro-2-(o-tolyl)-4-(1,1,1-trifluoropropan-2- |
| | yl)quinolin-6-yl)-3-(hydroxymethyl)-1H-1,2,4-triazol-5(4H)-one; |
| 37 | (R*)-4-Ethyl-1-(7-fluoro-2-(o-tolyl)-4-(1,1,1-trifluoropropan-2- |
| | yl)quinolin-6-yl)-3-(hydroxymethyl)-1H-1,2,4-triazol-5(4H)-one; |
| 38 | 4-Ethyl-1-(7-fluoro-4-isopropyl-2-(o-methyl-d3-phenyl)quinolin-6-yl)-3- |
| | (hydroxymethyl)-1H-1,2,4-triazol-5(4H)-one; and |
| 39 | 4-Ethyl-1-(7-fluoro-4-(prop-1-en-2-yl)-2-(o-tolyl)quinolin-6-yl)-3- |
| | (hydroxymethyl)-1H-1,2,4-triazol-5(4H)-one. |

An additional embodiment of the invention is a compound of Formula (I) having the Formula (IA):

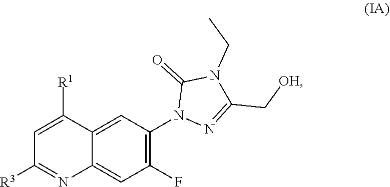

wherein

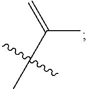 [SMILES: *C(=C)C]

R¹ is selected from the group consisting of: C₁₋₆alkyl, C₁₋₆haloalkyl and; and

R³ is selected from the group consisting of:

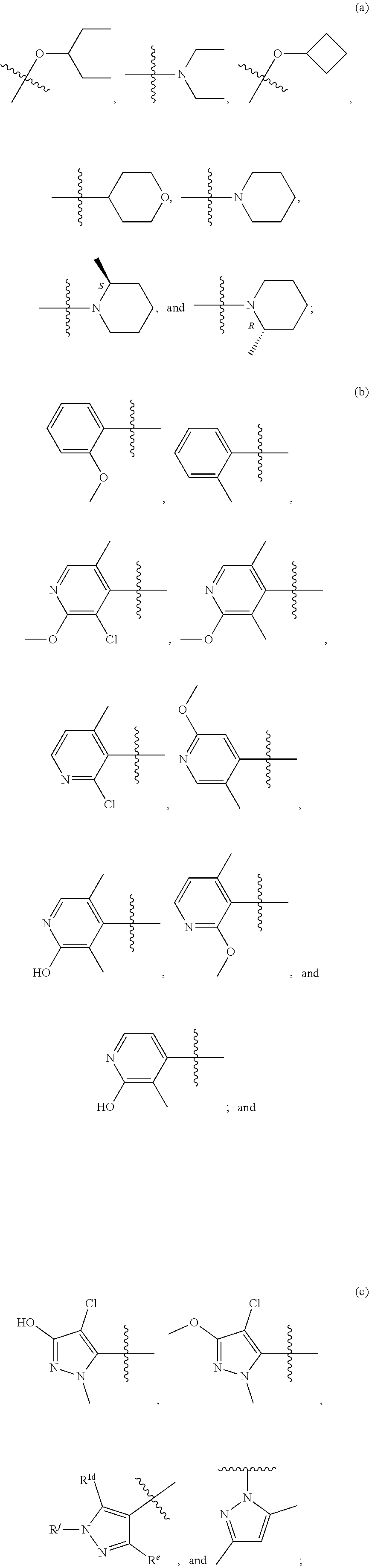

wherein

R^{d} is H, Cl, C₁₋₄alkyl or C₁₋₄haloalkyl;

R^{e} is halo, C₁₋₄alkyl, C₁₋₄haloalkyl or cyclopropyl; and

R^{f} is H.

An additional embodiment of the invention is a compound of Formula (I) having the Formula (IB):

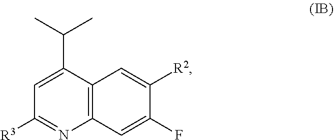

wherein

R² is

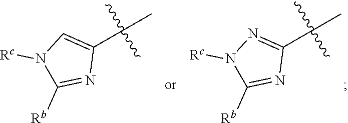

where
R^{b} is C₁₋₄alkyl substituted with OH;
and R^{c} is C₁₋₄alkyl;

R³ is

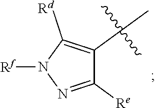

wherein
R^{d} is H, Cl, C₁₋₄alkyl or C₁₋₄haloalkyl;
R^{e} is halo, C₁₋₄alkyl, C₁₋₄haloalkyl or cyclopropyl; and
R_{f} is H.

An additional embodiment of the invention is a compound of Formula (I) having the Formula (IC):

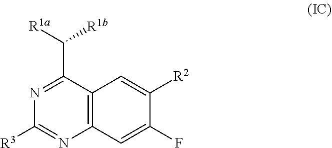

R^{1a} is CH₃;
R^{1b} is CH₃;
R² is

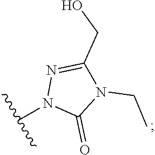 [SMILES: *n1nc(CO)n(CC)c1=O]

and
R³ is

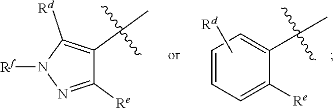

wherein
R^{d} is selected from the group consisting of: H, halo, C₁₋₆alkyl, and C₁₋₆haloalkyl;
R^{e} is selected from the group consisting of: halo, C₁₋₆alkyl, C₁₋₆haloalkyl, and cyclopropyl; and
R^{f} is H.

An additional embodiment of the invention is a compound of Formula (I) having the Formula (IA), wherein R¹ is CH(CH₃)₂; CH(CH₃)(CF₃) or

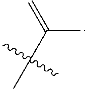 [SMILES: *C(=C)C]

An additional embodiment of the invention is a compound of Formula (I) having the Formula (IB), wherein R^{c} is CH₃.

An additional embodiment of the invention is a compound of Formula (I) having the Formula (IC), wherein R^{e} is C₁₋₄alkyl.

Also within the scope of the invention are enantiomers and diastereomers of the compounds of Formula (I) (as well as Formulas (IA), (IB), and (IC)). Also within the scope of the invention are pharmaceutically acceptable salts, N-oxides or solvates of the compounds of Formula (I) (as well as Formulas (IA), (IB), and (IC)). Also within the scope of the invention are the pharmaceutically acceptable prodrugs of compounds of Formula (I) (as well as Formulas (IA), (IB), and (IC)), and pharmaceutically active metabolites of the compounds of Formula (I) (as well as Formulas (IA), (IB), and (IC)).

Also within the scope of the invention are isotopic variations of compounds of Formula (I) (as well as Formulas (IA), (IB), and (IC)), such as, e.g., deuterated compounds of Formula (I). Also within the scope of the invention are the pharmaceutically acceptable salts, N-oxides or solvates of the isotopic variations of the compounds of Formula (I) (as well as Formulas (IA), (IB), and (IC)). Also within the scope of the invention are the pharmaceutically acceptable prodrugs of the isotopic variations of the compounds of Formula (I) (as well as Formulas (IA), (IB), and (IC)), and pharmaceutically active metabolites of the isotopic variations of the compounds of Formula (I) (as well as Formulas (IA), (IB), and (IC)).

Even though the compounds of embodiments of the present invention (including their pharmaceutically acceptable salts and pharmaceutically acceptable solvates) can be administered alone, they will generally be administered in admixture with a pharmaceutically acceptable carrier, a pharmaceutically acceptable excipient and/or a pharmaceutically acceptable diluent selected with regard to the intended route of administration and standard pharmaceutical or veterinary practice.

Thus, particular embodiments of the present invention are directed to pharmaceutical and veterinary compositions comprising compounds of Formula (I) and at least one pharmaceutically acceptable carrier, pharmaceutically acceptable excipient, and/or pharmaceutically acceptable diluent. By way of example, in the pharmaceutical compositions of embodiments of the present invention, the compounds of Formula (I) may be admixed with any suitable binder(s), lubricant(s), suspending agent(s), coating agent(s), solubilizing agent(s), and combinations thereof.

An embodiment of the invention relates to a pharmaceutical composition comprising an effective amount of at least one compound selected from compounds of Formula (I), and pharmaceutically acceptable salts, isotopes, tautomers, N-oxides, solvates, and stereoisomers thereof, in accordance with any embodiment described herein; and at least one pharmaceutically acceptable excipient.

An additional embodiment of the invention is a pharmaceutical composition comprising:
(A) an effective amount of at least one compound selected from compounds of Formula (I)

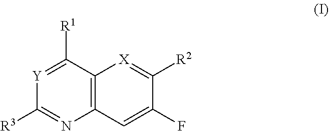

wherein
X is CH or N;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆-haloalkyl substituted with OH, or OCH₃; C₃₋₆cycloalkyl; C₃₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of:

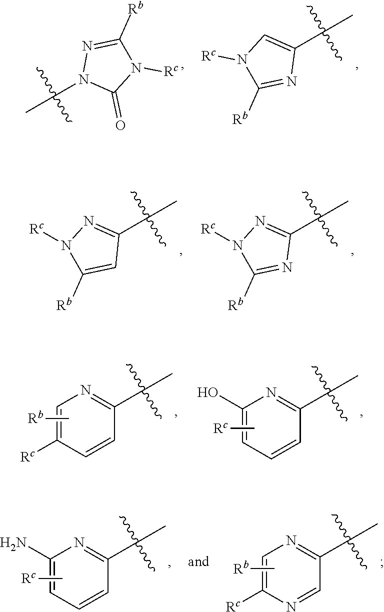

where
R^{b} is C₁₋₆alkyl substituted with a member selected from the group consisting of: OH, halo, CN, OC₁₋₆alkyl, OC₁₋₄haloalkyl and OC₃₋₆cycloalkyl;
R^{c} is selected from the group consisting of: C₁₋₆alkyl, C₁₋₆haloalkyl, C₃₋₆cycloalkyl, and tetrahydro-2H-pyranyl; and
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

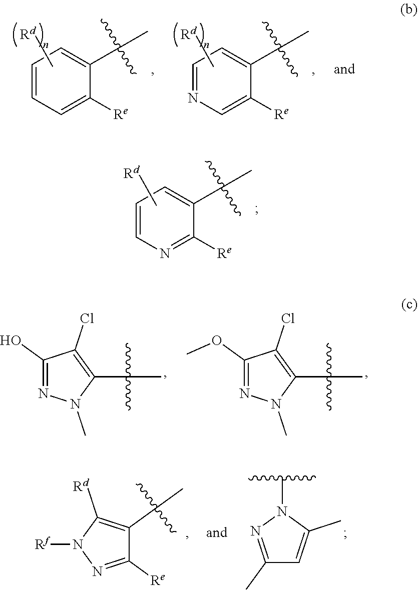

where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
   or pharmaceutically acceptable salts, isotopes, tautomers, N-oxides, solvates, or stereoisomers of a compound of Formula (I);
   and (B) at least one pharmaceutically acceptable excipient.

An additional embodiment of the invention is a pharmaceutical composition comprising an effective amount of a compound shown in Table 1 (e.g., a compound selected from Examples 1-39), or a pharmaceutically acceptable salt, isotope, N-oxide, solvate, or stereoisomer of the compound of Table 1, a pharmaceutically acceptable prodrug of the compound of Table 1, or a pharmaceutically active metabolite of the compound of Table 1; and at least one pharmaceutically acceptable excipient.

Solid oral dosage forms such as, tablets or capsules, containing one or more compounds of the present invention may be administered in at least one dosage form at a time, as appropriate. It is also possible to administer the compounds in sustained release formulations.

Additional oral forms in which the present inventive compounds may be administered include elixirs, solutions, syrups, and suspensions; each optionally containing flavoring agents and coloring agents.

Alternatively, one or more compounds of Formula (I) can be administered by inhalation (intratracheal or intranasal) or in the form of a suppository or pessary, or they may be applied topically in the form of a lotion, solution, cream, ointment or dusting powder. For example, they can be incorporated into a cream comprising, consisting of, and/or consisting essentially of an aqueous emulsion of polyethylene glycols or liquid paraffin. They can also be incorporated, at a concentration of between about 1% and about 10% by weight of the cream, into an ointment comprising, consisting of, and/or consisting essentially of a wax or soft paraffin base together with any stabilizers and preservatives as may be required. An alternative means of administration includes transdermal administration by using a skin or transdermal patch.

The pharmaceutical compositions of the present invention (as well as the compounds of the present invention alone) can also be injected parenterally, for example, intracavemosally, intravenously, intramuscularly, subcutaneously, intradermally, or intrathecally. In this case, the compositions will also include at least one of a suitable carrier, a suitable excipient, and a suitable diluent.

For parenteral administration, the pharmaceutical compositions of the present invention are best used in the form of a sterile aqueous solution that may contain other substances, for example, enough salts and monosaccharides to make the solution isotonic with blood.

For buccal or sublingual administration, the pharmaceutical compositions of the present invention may be administered in the form of tablets or lozenges, which can be formulated in a conventional manner.

By way of further example, pharmaceutical compositions containing at least one of the compounds of Formula (I) as the active ingredient can be prepared by mixing the compound(s) with a pharmaceutically acceptable carrier, a pharmaceutically acceptable diluent, and/or a pharmaceutically acceptable excipient according to conventional pharmaceutical compounding techniques. The carrier, excipient, and diluent may take a wide variety of forms depending upon the desired route of administration (e.g., oral, parenteral, etc.). Thus, for liquid oral preparations such as, suspensions, syrups, elixirs and solutions, suitable carriers, excipients and diluents include water, glycols, oils, alcohols, flavoring agents, preservatives, stabilizers, coloring agents and the like; for solid oral preparations such as, powders, capsules, and tablets, suitable carriers, excipients and diluents include starches, sugars, diluents, granulating agents, lubricants, binders, disintegrating agents and the like. Solid oral preparations also may be optionally coated with substances such as, sugars, or be enterically coated so as to modulate the major site of absorption and disintegration. For parenteral administration, the carrier, excipient and diluent will usually include sterile water, and other ingredients may be added to increase solubility and preservation of the composition. Injectable suspensions or solutions may also be prepared utilizing aqueous carriers along with appropriate additives such as, solubilizers and preservatives.

According to particular embodiments, a therapeutically effective amount of a compound of Formula (I) or a pharmaceutical composition thereof may comprise a dose range from about 0.1 mg to about 3000 mg, or any particular amount or range therein, in particular from about 1 mg to about 1000 mg, or any particular amount or range therein, or, more particularly, from about 10 mg to about 500 mg, or any particular amount or range therein, of active ingredient in a regimen of about 1 to about (4×) per day for an average (70 kg) human; although, it is apparent to one skilled in the art that the therapeutically effective amount for a compound of Formula (I) will vary as will the diseases, syndromes, conditions, and disorders being treated.

For oral administration, a pharmaceutical composition may be provided in the form of one or more tablets containing about 1.0, about 10, about 50, about 100, about 150, about 200, about 250, or about 500 milligrams of a compound of Formula (I).

An embodiment of the present invention is directed to a pharmaceutical composition for oral administration, comprising a compound of Formula (I) in an amount of from about 1 mg to about 500 mg.

Advantageously, a compound of Formula (I) may be administered in a single daily dose, or the total daily dosage may be administered in divided doses of two, three and (4×) daily.

Optimal dosages of a compound of Formula (I) to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

Compounds of Formula (I) may be administered in any of the foregoing compositions and dosage regimens or by means of those compositions and dosage regimens established in the art whenever use of a compound of Formula (I) is administered to a subject in need thereof.

According to particular embodiments, one or more compounds of Formula (I) are useful in methods for treating, ameliorating and/or preventing a disease, a syndrome, a condition, or a disorder that is affected by the inhibition of DHODH enzymatic activity.

An additional embodiment of the invention relates to the use of compounds of Formula (I), e.g., by inhibiting dihydroorotate oxygenase enzyme activity, in treating disorders like inflammatory disorders, autoimmune disorders, or cancer;

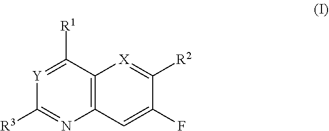

wherein
X is CH or N;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₃₋₆cycloalkyl; C₁₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of:

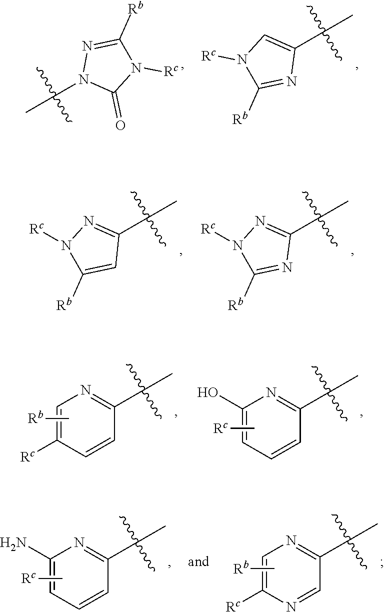

where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₁₋₆cycloalkyl, and tetrahydro-2H-pyranyl; and
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

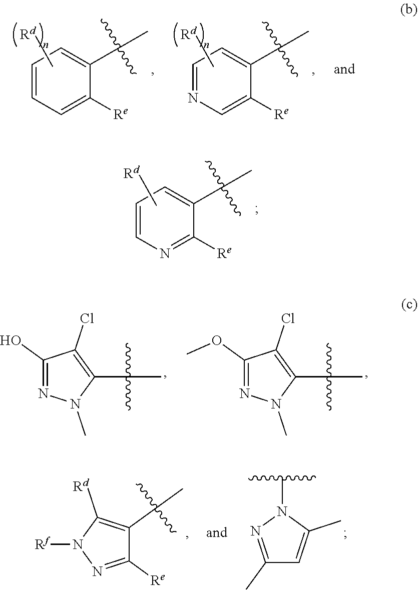

where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₁₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
   or pharmaceutically acceptable salts, isotopes, tautomers, N-oxides, solvates, or stereoisomers thereof.

In a further aspect the present invention provides a method for inhibiting or altering Dihydroorotate Dehydrogenase (DHODH) enzymatic activity, the method comprising contacting DHODH with any compound of Formula (I), aspect or embodiment disclosed herein, thereby inhibiting or otherwise altering DHODH enzymatic activity.

An additional embodiment of the present invention provides methods for treating diseases, disorders, or medical conditions mediated or otherwise affected by dihydroorotate dehydrogenase (DHODH) enzyme activity comprising administering a compound of Formula (I) to a subject in need thereof.

As used herein, the term “DHODH inhibitor” may refer to an agent that inhibits or reduces DHODH activity.

In one embodiment, the term “therapeutically effective amount” (or “effective amount”) refers to the amount of a compound of the present invention that, when administered to a subject, is effective to (1) at least partially alleviate, inhibit, prevent, and/or ameliorate a condition, or a disorder or a disease (i) mediated by DHODH enzymatic activity; or (ii) associated with DHODH enzymatic activity; or (iii) characterized by activity (normal or abnormal) of DHODH enzyme; or (2) reduce or inhibit the activity of DHODH enzyme; or (3) reduce or inhibit the expression of DHODH; or (4) modify the protein levels of DHODH. Without being bound by a particular theory, DHODH inhibitors are believed to act by inhibiting nucleic acid synthesis, cell cycle arrest or altering post-translational glycosylation of proteins involved in regulating myeloid differentiation within progenitor tumor cells.

An additional embodiment of the invention is a method of treating a subject suffering from or diagnosed with a disease, disorder, or medical condition mediated or otherwise affected by DHODH enzymatic activity, comprising administering to a subject in need of such treatment an effective amount of at least one compound selected from compounds of Formula (I) (as well as Formulas (IA), (IB), and (IC), such as a compound of Table 1), enantiomers and diastereomers of the compounds of Formula (I) (as well as Formulas (IA), (IB), and (IC), such as a compound of Table 1), isotopic variations of the compounds of Formula (I) (as well as Formulas (IA), (IB), and (IC), such as a compound of Table 1), and pharmaceutically acceptable salts of all of the foregoing. Stated another way, according to an embodiment, a method of treating a subject suffering from or diagnosed with a disease, disorder, or medical condition comprises inhibiting or otherwise altering dihydroorotate oxygenase enzyme activity in the subject by administering to the subject an effective amount of at least one compound selected from compounds of Formula (I) (as well as Formulas (IA), (IB), and (IC), such as a compound of Table 1).

In another embodiment, inhibitors of DHODH of the present invention may be used for the treatment of immunological diseases including, but not limited to, autoimmune and inflammatory disorders, e.g. arthritis, inflammatory bowel disease, gastritis, ankylosing spondylitis, ulcerative colitis, pancreatitis, Crohn's disease, celiac disease, multiple sclerosis, systemic lupus erythematosus, lupus nephritis, rheumatic fever, gout, organ or transplant rejection, chronic allograft rejection, acute or chronic graft-versus-host disease, dermatitis including atopic, dermatomyositis, psoriasis, Behcet's diseases, uveitis, myasthenia gravis, Grave's disease, Hashimoto thyroiditis, Sjogren's syndrome, blistering disorders, antibody-mediated vasculitis syndromes, immune-complex vasculitides, allergic disorders, asthma, bronchitis, chronic obstructive pulmonary disease (COPD), cystic fibrosis, pneumonia, pulmonary diseases including edema, embolism, fibrosis, sarcoidosis, hypertension and emphysema, silicosis, respiratory failure, acute respiratory distress syndrome, BENTA disease, berylliosis, and polymyositis.

As used herein, unless otherwise noted, the term “affect” or “affected” (when referring to a disease, disorder, or medical condition that is affected by the inhibition or alteration of DHODH enzymatic activity) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

An additional embodiment of the invention provides a method of treatment of cancer comprising administering to a subject in need thereof, a therapeutically effective amount of a compound of Formula (I), or a pharmaceutically acceptable salt, isotope, N-oxide, solvate, or stereoisomer thereof.

According to an embodiment, the cancer is selected from but not limited to, lymphomas, leukemias, carcinomas, and sarcomas.

An additional embodiment of the invention provides the use of a compound of Formula (I), or a pharmaceutically acceptable salt, isotope, N-oxide, solvate, or stereoisomer thereof, for the treatment of one or more cancer types.

According to particular embodiments, the uses and methods of treatment described herein are directed to the treatment of cancer, wherein the cancer is selected from but not limited to:
leukemias including but not limited to acute lymphoblastic leukemia (ALL), acute myeloid leukemia (AML), (acute) T-cell leukemia, acute monocytic leukemia, acute promyelocytic leukemia (APL), bisphenotypic B myelomonocytic leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia,
lymphomas including but not limited to AIDS-related lymphoma, Hodgkin lymphoma, non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell DLBCL, double-hit lymphoma and double-expressor lymphoma; anaplastic large cell lymphoma, marginal B cell lymphoma and primary mediastinal B-cell lymphoma, immunoblastic large cell lymphoma, Burkitt lymphoma, follicular lymphoma, hairy cell leukemia, Hodgkin's disease, mantle cell lymphoma (MCL), lymphoplasmatic lymphoma, precursorB-lymphoblastic lymphoma, lymphoma of the central nervous system, small lymphocytic lymphoma (SLL) and chronic lymphocytic leukemia (CLL); T-cell NHL such as precursor T-lymphoblastic lymphoma/leukemia, peripheral T-cell lymphoma (PTCL), cutaneous T-cell lymphoma (CTCL), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma
sarcomas including but not limited to sarcoma of the soft tissue, gliosarcoma, osteosarcoma, malignant fibrous histiocytoma, lymphosarcoma, and rhabdomyosarcoma;
   and
other cancers, such as solid tumors, including but not limited to breast cancer, colorectal carcinoma, gastric cancer, gliosarcoma, head & neck cancer, hepatocellular carcinoma, lung cancer, multiple myeloma, neuroblastoma, ovarian cancer, pancreatic cancer, prostate cancer, renal cell carcinoma and sarcoma.

In an embodiment, cancers that may benefit from a treatment with inhibitors of DHODH of the present invention include, but are not limited to, lymphomas, leukemias, carcinomas, and sarcomas, e.g. non-Hodgkin's lymphoma, diffuse large B-cell lymphoma (DLBCL), mantle cell lymphoma (MCL), follicular lymphoma (FL), marginal zone lymphoma, T-cell lymphoma, Hodgkin's lymphoma, Burkitt's lymphoma, multiple myeloma, brain (gliomas), glioblastomas, breast cancer, colorectal/colon cancer, prostate cancer, lung cancer including non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head & neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

In another embodiment of the present invention, the compounds of the present invention may be employed in combination with one or more other medicinal agents, more particularly with one or more anti-cancer agents, e.g. chemotherapeutic, antiproliferative or immunomodulating agents, or with adjuvants in cancer therapy, e.g. immunosuppressive or anti-inflammatory agents. Additional non-limiting examples of anti-cancer agents that may be administered in combination with a compound of the present invention include biologic compounds, such as monoclonal antibodies (e.g., that mediate effector function upon binding to cancer cell-associated antigens, or block interaction of a receptor expressed on cancer cells with a soluble or cell bound ligand), bispecific antibodies that mediate immune cell redirection, etc. According to an embodiment, a method of treating cancer comprises administering an effective amount of a compound of the present invention (e.g., selected from compounds of Formula (I), such as a compound shown in Table 1, pharmaceutically acceptable salts, isotopes, tautomers, N-oxides, solvates, and stereoisomers thereof) and an effective amount of one or more additional anti-cancer agents, wherein the method comprises administering the compound of the present invention and the additional anti-cancer agent(s) either simultaneously (e.g., as part of the same pharmaceutical composition) or sequentially. According to an embodiment, a pharmaceutical composition comprises an effective amount of a compound of the present invention (e.g., selected from compounds of Formula (I), such as a compound shown in Table 1, pharmaceutically acceptable salts, isotopes, tautomers, N-oxides, solvates, and stereoisomers thereof), an effective amount of one or more additional anti-cancer agents, and optionally one or more excipients.

An additional embodiment of the invention provides the use of a compound of Formula (I), or pharmaceutically acceptable salts, isotopes, tautomers, N-oxides, solvates, or stereoisomers thereof, as part of chemotherapeutic regimens for the treatment of cancers, lymphomas and leukemias alone or in combination with classic antitumoral compounds well known by the one skilled in the art.

### General Synthetic Methods

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Abbreviations used in the instant specification, particularly the schemes and examples, are as follows:
ACN acetonitrile
AcOH glacial acetic acid
aq. Aqueous
B₂Pin₂ Bis(pinacolato)diboron
Bn or Bzl benzyl
Boc tert-butyloxycarbonyl
conc. Concentrated
CPhos Chloro[(2-dicyclohexylphosphino-2′,6′-bis(N,N-dimethylamino)-1,1′-biphenyl)-2-(2′-amino-1,1′-biphenyl)]palladium(II)
DCC N,N′-dicyclohexyl-carbodiimide
DCE dichloroethane
DCM dichloromethane
DIPEA or DIEA diisopropyl-ethyl amine
DMA dimethylaniline
DMAP 4-dimethylaminopyridine
DME dimethoxyethane
DMF N,N-dimethylformamide
DMSO dimethylsulfoxide
EA ethyl acetate
EDCI 1-ethyl-3-(3-dimethylaminopropyl) carbodiimide
ESI electrospray ionization
EtOAc or EA ethyl acetate
EtOH ethanol
GCMS gas chromatography-mass spectrometry
h or hr(s) hour or hours
HPLC high performance liquid chromatography
KHMDS Potassium bis(trimethylsilyl)amide
LAH Lithium aluminum hydride
LiHMDS Lithium bis(trimethylsilyl)amide
mCPBA meta-Chloroperoxybenzoic acid
MeOH methanol
MHz megahertz
min minute or minutes
MS mass spectrometry
NaBH₄ sodium borohydride
NaHMDS Sodium bis(trimethylsilyl)amide
NBS N-bromosuccinimide
NMR nuclear magnetic resonance
Pd-118 [1,1′-Bis(di-tert-butylphosphino)ferrocene]dichloropalladium(II)
PE petroleum ether
PEPPSI-iPr [1,3-Bis(2,6-Diisopropylphenyl)imidazol-2-ylidene](3-chloropyridyl)palladium(II) dichloride
RP reverse-phase
rt or RT room temperature
R& retention time
Sec second or seconds
TBDPS tert-Butyldiphenylchlorosilane
TBS tert-Butyldimethylsilyl
TES triethylsilane
TIPS triisopropylsilane
TEA or Et₃N triethylamine
TFA trifluoroacetic acid
THF tetrahydrofuran
TLC thin layer chromatography
XPhos (2-Dicyclohexylphosphino-2′,4′,6′-triisopropyl-1,1′-biphenyl)[2-(2′-amino-1,1′-biphenyl)]palladium(II) methanesulfonate

### PREPARATIVE EXAMPLES

Exemplary compounds useful in methods of the invention will now be described by reference to the illustrative synthetic schemes for their general preparation below and the specific examples to follow.

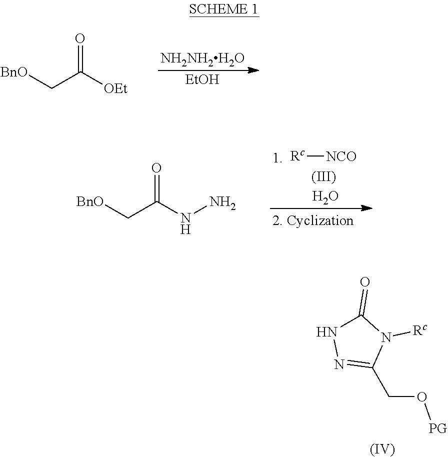

According to SCHEME 1, a 1,2,4-triazol-5 (4H)-one compound of formula (IV), where PG is Bn, is prepared from ethyl 2-(benzyloxy)acetate in three steps. In a first step 2-(benzyloxy)acetohydrazide is prepared by the reaction of ethyl 2-(benzyloxy)acetate with hydrazine hydrate, in a suitable solvent such as EtOH, and the like; at temperatures ranging from 70-85° C. Reaction of the hydrazide with an isocyanate of formula (III), where R^{c} is C₁₋₆alkyl, in a suitable solvent such as water, and the like; provides the corresponding semicarbazide. Subsequent cyclization of the semicarbazide with a suitable base such as NaOH, in a suitable solvent such as water, provides a compound of formula (IV), where PG is Bn.

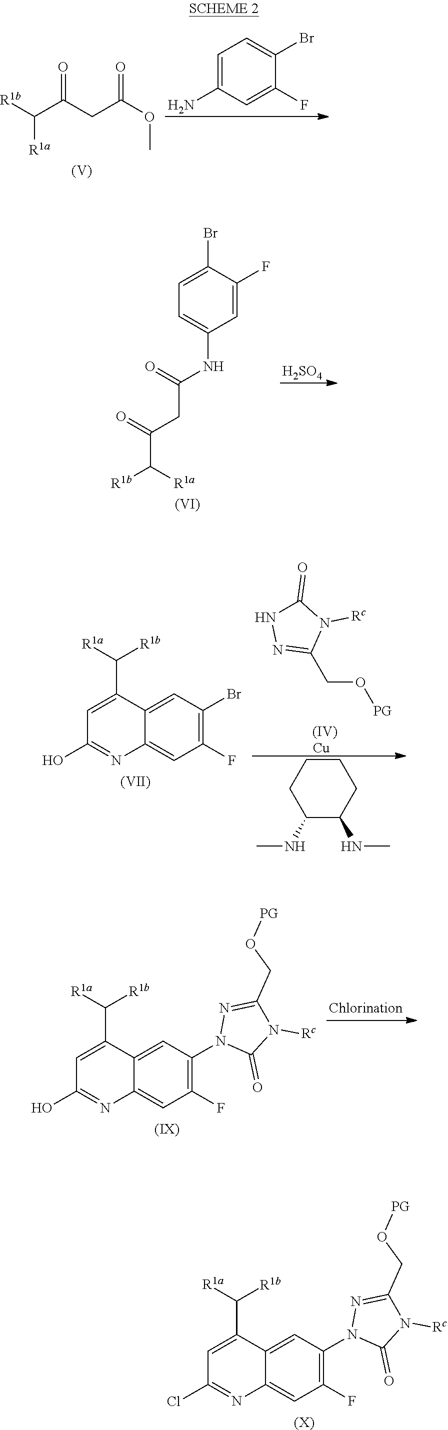

According to SCHEME 2, condensation of 4-bromo-3-fluoro aniline with a commercially available or synthetically accessible compound of formula (V), where R^{1a} and R^{1b} are as defined in claim 1; in the presence of a suitable base such as Et₃N, and the like; in a suitable solvent such as THF, toluene, ACN, and the like, preferably toluene; affords a compound of formula (VI). In a Knorr reaction, a compound of formula (VI) is cyclized employing a strong acid such as H₂SO₄, polyphosphoric acid, and the like; at temperatures ranging from 25° C. to 60° C.; for a period of 24-48 hours; to afford a hydroxy quinoline compound of formula (VII). A compound of formula (VII), is reacted with a commercially available or synthetically accessible compound of formula (IV) where R^{c} is as defined in claim 1, and PG is benzyl; employing copper catalyzed arylation conditions to provide a compound of formula (IX). For example, reaction of compound of formula (VII) with a compound of formula (IV); a Cu(I) or Cu(II) salt such as CuI or CuBr; with or without KI as an additive; a ligand such as trans-N,N-dimethylcyclohexane-1,2-diamine, 1,2-cyclohexanediamine, N,N′-dimethylglycine, 2-((2,6-dimethylphenyl)amino)-2-oxoacetic acid and the like; a base such as Cs₂CO₃, K₂CO₃, K₃PO₄, K₂HPO₄, KHCO₃, Na₂CO₃, NaHCO₃, and the like; in a suitable solvent such as dioxane, and the like; at temperatures ranging from 90 to 110° C.; for a period of about 16 to 24 hours; provides a compound of formula (IX).

Chlorination of a compound of formula (IX) is achieved employing conditions known to one skilled in the art, for example, a compound of formula (IX) is treated with a chlorinating agent such as POCl₃, and the like, at temperatures ranging from 70-90° C., to provide a chloro-quinoline compound of formula (X).

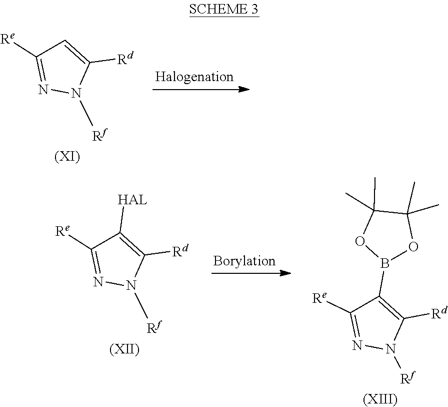

According to SCHEME 3, a commercially available or synthetically accessible compound of formula (XI), where R^{d} and R^{e} are as described in claim 1, and R is H; is halogenated under conditions known to one skilled in the art. For example, 3-chloro-5-methyl-1H-pyrazole is halogenated using a reagent such as NBS, and like, in a suitable solvent such as DCM, DMF, and the like, at a temperature ranging from 0° C. to rt, to provide a compound of formula (XII), where HAL is Br. A compound of formula (XII), is treated with a borylating agent such as bis(pinacolato)diboron; in the presence of a palladium catalyst such as Pd(dppf)Cl₂, and the like; a suitable base such as potassium acetate; employing conventional heating, at a temperature ranging from 75° C. to 100° C.; in a suitable solvent such as 1,4-dioxane, and the like; to provide a compound of formula (XIII).

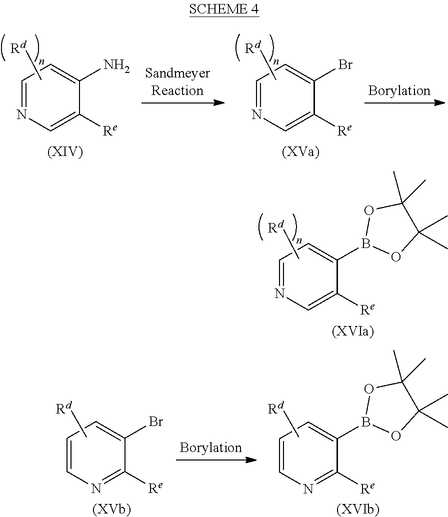

According to SCHEME 4, commercially available or synthetically accessible compound of formula (XIV), where R^{d} and R^{e} are as described in claim 1, and n is 1 or 2; is converted to a bromide compound of formula (XV), using a Sandmeyer reaction conditions known to one skilled in the art. For example, a compound of formula (XIV), R^{d} and R^{e} are as described in claim 1, and n is 1 or 2; is reacted with tert-butyl nitrite, isoamyl nitrite or sodium nitrite, and the like; in a suitable solvent such as acetonitrile (MeCN), or 1,4-dioxane, and the like; CuBr; at temperatures ranging from room temperature to 70° C.; for a period of 16 to 24 h; to provide a compound of formula (XVa). A compound of formula (XVa) or (XVb), where R^{d} and R^{e} are as described in claim 1, and n is 1 or 2; is borylated employing conditions known to one skilled in the art or as previously described to afford compounds of formula (XVIa) or (XVIb).

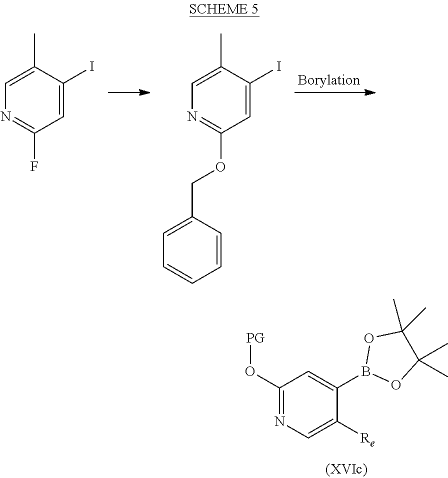

According to SCHEME 5, phenylmethanol is reacted with a suitable base such as NaH, and the like; 2-fluoro-4-iodo-5-methylpyridine; in a suitable solvent such as THF, and the like; at temperatures ranging from 0° C. to room temperature; for a period of 1-3 hrs; to provide 2-(benzyloxy)-4-iodo-5-methylpyridine. 2-(Benzyloxy)-4-iodo-5-methylpyridine is reacted under borylation conditions known to one skilled in the art, or as previously described to provide a compound of formula (XVIc), where PG is benzyl.

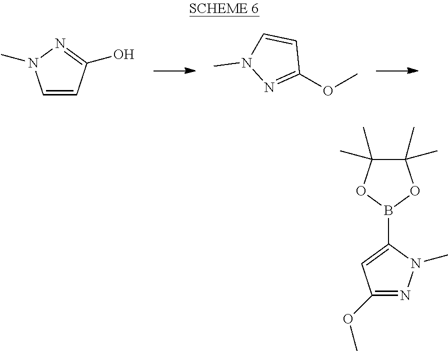

According to SCHEME 6, 1-methyl-1H-pyrazol-3-ol is alkylated with a suitable alkylating agent such as iodomethane, and the like; a suitable base such K2CO3, and the like; in a suitable solvent such as acetonitrile, and the like; at room temperature; to provide 3-methoxy-1-methyl-1H-pyrazole. 3-Methoxy-1-methyl-1H-pyrazole is borylated employing (1,5-cyclooctadienexmethoxy)iridium(I) dimer, 4,4′-di-tert-butyl-2,2′-bipyridine, 4,4,5,5-tetramethyl-1,3,2-dioxaborolane, in a suitable solvent such as hexanes, to provide 3-methoxy-1-methyl-5-(4,4,5,5-tetramethyl-1,3,2-dioxaborolan-2-yl)-1H-pyrazole.

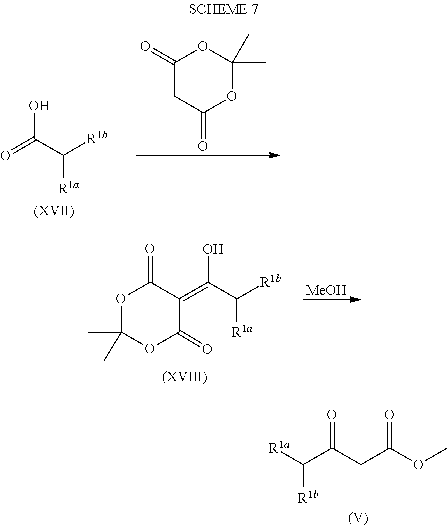

According to SCHEME 7, a compound of formula (XVIII) is prepared from a commercially available or synthetically accessible compound of formula (XVII), where R^{1a} is C₁₋₆haloalkyl and R^{1b} is CH₃. A compound of formula (XVII) is acylated with 2,2-dimethyl-1,3-dioxane-4,6-dione; a suitable coupling reagent such as DCC, DIC, and the like, preferably DCC; in a suitable solvent such as DCM, DMF, THF, and the like, preferably DCM; at temperatures ranging from 0° C. to 20° C.; for a time period of 16 hours; to provide a compound of formula (XVIII). A compound of formula (XVIII) undergoes a pericyclic reaction and pyrolysis in the presence of an alcohol, for example methanol; at refluxing temperatures; for a period of 5 hours; to afford a compound of formula (V).

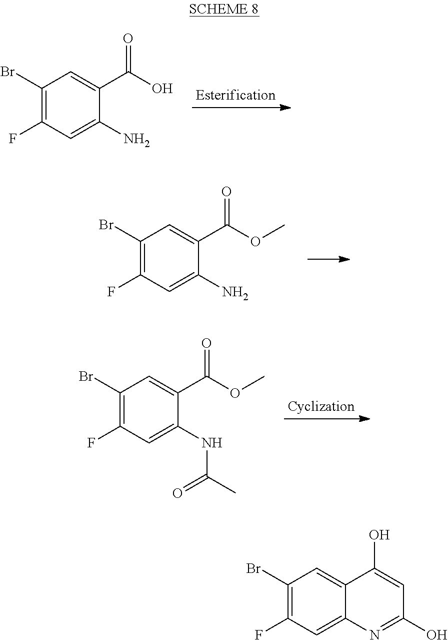 [SMILES: Nc1cc(F)c(Br)cc1C(=O)O ; COC(=O)c1cc(Br)c(F)cc1N ; COC(=O)c1cc(Br)c(F)cc1NC(C)=O ; Oc1cc(O)c2cc(Br)c(F)cc2n1]

According to SCHEME 8, 2-amino-5-bromo-4-fluorobenzoic acid is esterified with an alcohol such as MeOH, and the like; thionyl chloride; and DMF; to provide methyl 2-amino-5-bromo-4-fluorobenzoate. Methyl 2-amino-5-bromo-4-fluorobenzoate is reacted with acetic anhydride; DMAP; and pyridine; to provide methyl 2-(N-acetylacetamido)-5-bromo-4-fluorobenzoate. Methyl 2-(N-acetylacetamido)-5-bromo-4-fluorobenzoate is reacted with Na₂CO₃; in a suitable solvent suitable solvent such as MeOH, and the like; at 20° C.; for a period of 1 hr; to provide methyl 2-acetamido-5-bromo-4-fluorobenzoate. Methyl 2-acetamido-5-bromo-4-fluorobenzoate is cyclized in the presence of a suitable base such as LiHMDS, KHMDS, and the like; in a suitable solvent such as THF, toluene, ACN, and the like, preferably THF; at temperatures ranging from −78° C. to 25° C. for a period of 3 hours; to provide 6-bromo-7-fluoroquinoline-2,4-diol.

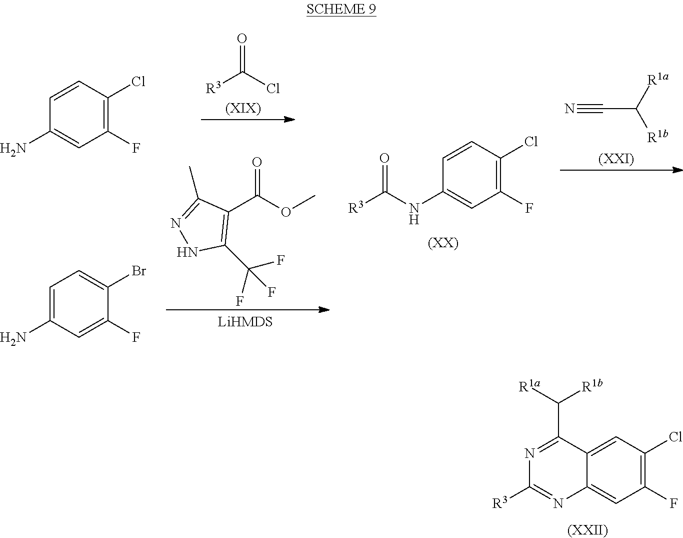

According to SCHEME 9, condensation of 4-chloro-3-fluoroaniline with a commercially available or synthetically accessible acid chloride of formula (XIX), where R³ is as defined in claim 1; in the presence of a suitable base such as NEt₃, and the like; in a suitable solvent such as THF, DCM, DMF, and the like, preferably DCM; affords a compound of formula (XX). In an alternate method, 4-bromo-3-fluoroaniline is reacted with methyl 3-methyl-5-(trifluoromethyl)-1H-pyrazole-4-carboxylate; a suitable base such as LiHMDS; in a suitable solvent such as DCM, and the like; to provide a compound of formula (XX), where R³ is 3-methyl-5-(trifluoromethyl)-1H-pyrazol-4-yl.

A compound of formula (XX), is reacted with a commercially available or synthetically accessible nitrile of formula (XXI) where R^{1a} and R^{1b} are CH₃; employing 2-chloropyridine and Tf₂O, in a suitable solvent such as DCM or DCE; at temperatures ranging from −78° C. to 140° C.; employing microwave heating; for a period of about 20 minutes to 16 hours; to provide a quinazoline of formula (XXII).

4-Bromo-3-fluoroaniline is reacted with methyl 3-methyl-5-(trifluoromethyl)-1H-pyrazole-4-carboxylate; a suitable base such as LiHMDS; in a suitable solvent such as DCM, and the like; to provide a compound of formula (XX), where R³ is 3-methyl-5-(trifluoromethyl)-1H-pyrazol-4-yl.

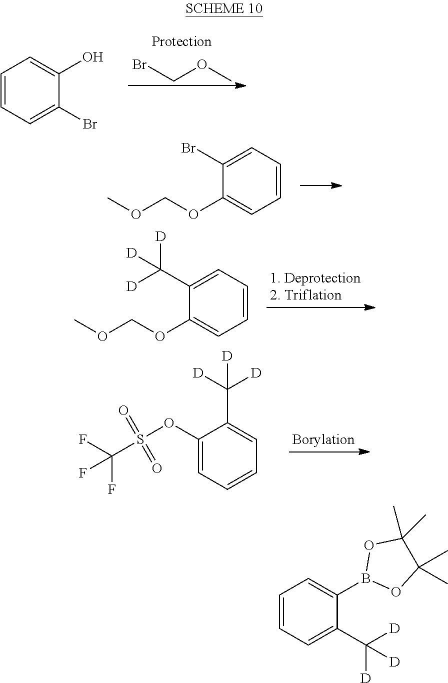

According to SCHEME 10, 2-bromophenol is reacted with bromo(methoxy)methane, in a suitable solvent such as DCM, and the like; at temperatures ranging from 0° C. to room temperature; for a period of 12-18 hr; to provide the methoxymethyl ether (MOM) compound 1-bromo-2-(methoxymethoxy)benzene. 1-Bromo-2-(methoxymethoxy)benzene is reacted with CD₃I; in a suitable solvent such as THF, and the like; to provide 1-(methoxymethoxy)-2-methyl-d₃-benzene. o-Methyl-d₃-phenyl trifluoromethanesulfonate is prepared in two steps from 1-(methoxymethoxy)-2-methyl-d₃-benzene. In a first step, the MOM protecting group is removed from 1-(methoxymethoxy)-2-methyl-d₃-benzen, employing conditions known to one skilled in the art, employing TsOH, in s suitable solvent such as MeOH, at 50° C.; to provide o-methyl-d₃-phenol. Subsequent derivation of o-methyl-d₃-phenol, with a sulfonate-based leaving group such as trifluoromethanesulfonyl (triflate), is achieved by is by reaction with a triflating agent such as trifluoromethanesulfonic anhydride (Tf₂O), a base such as triethylamine (TEA), pyridine, and the like, to provide o-methyl-d₃-phenyl trifluoromethanesulfonate. Borylation of o-methyl-d₃-phenyl trifluoromethanesulfonate is achieved employing conditions previously described or according to methods known to one skilled in the art to provide 4,4,5,5-tetramethyl-2-(o-methyl-d₃-phenyl)-1,3,2-dioxaborolane.

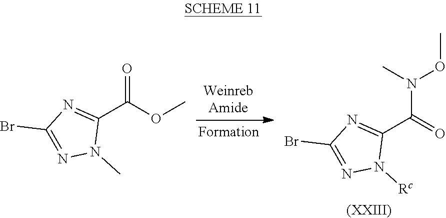

According to SCHEME 11, methyl 3-bromo-1-methyl-1H-1,2,4-triazole-5-carboxylate is reacted with N,O-dimethylhydroxylamine hydrochloride; bis(trimethylaluminum)-1,4-diazabicyclo[2.2.2]octane adduct; in a suitable solvent such as THF, and like; at temperatures ranging from 40° C. to 80° C.; for a period of about 18 hrs; to provide a compound of formula (XXIII), where R^{c} is CH₃.

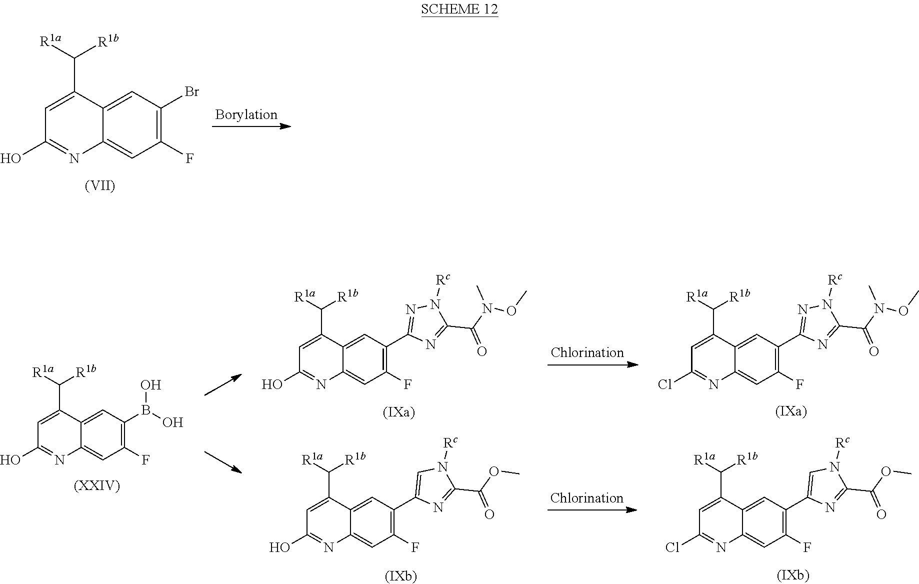

According to SCHEME 12, a compound of formula (VII), where R^{1a} and R^{1b} are as defined in claim 1; is borylated employing methods known to one skilled in the art or as previously described; to afford a compound of formula (XXIV). A compound of formula (XXIV) is reacted with a Weinreb amide compound of formula (XXIII), where R^{c} is CH₃ or methyl 4-bromo-1-methyl-1H-imidazole-2-carboxylate; in a metal mediated cross coupling reaction, in the presence of a palladium catalyst such as Pd-118, [1,1-bis(di-tert-butylphosphino)ferrocene]dichloropalladium(II) (Pd(dtbpf)Cl₂), XPhos Pd G3, Pd(PPh₃)₄, bis(triphenylphosphine)palladium(II)chloride (PdCl₂(PPh₃)₂), bis(diphenylphosphino)ferrocene]dichloropalladium(II) complex with dichloromethane, (2-dicyclohexylphosphino-2′,6′-diisopropoxy-1,1′-biphenyl)[2-(2′-amino-1,1′-biphenyl)]palladium(II) methanesulfonate (RuPhos Pd G3), [1,1′-bis(diphenylphosphino)ferrocene]dichloropalladium(II) (Pd(dppf)Cl₂), and the like; a base such as KF, K₃PO₄, aq. Na₂CO₃, Cs₂CO₃, and the like; in a suitable solvent such as 1,4-dioxane, dimethylformamide (DMF), acetonitrile (ACN), water, or a mixture thereof; at temperatures ranging from 60 to 120° C.; for a period of about 16 to 48 hours; to provide a compound of formula (IXa) or a compound of formula (IXb). Chlorination of compounds of formula (IXa) and (IXb) is achieved employing methods known to one skilled in the art, or as previously described to afford compounds of formula (Xa) and (Xb).

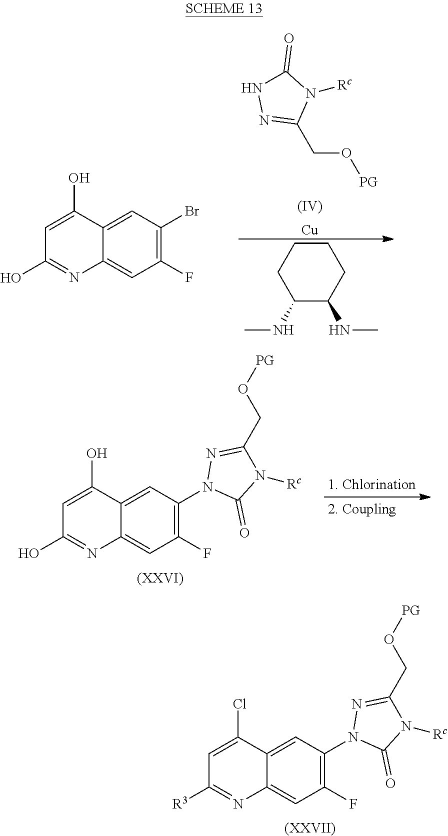

According to SCHEME 13, 6-bromo-7-fluoroquinoline-2,4-diol is reacted with a compound of formula (IV), where R^{c} is C₁₋₄alkyl and PG is benzyl; employing conditions previously described to afford a compound of formula (XXVI). A compound of formula (XXVI) is chlorinated employing conditions known to one skilled in the art, or as previously described. Subsequent coupling with a suitably substituted commercially available or synthetically accessible aryl or heteroaryl boronic acid or boronate ester, employing conditions known to one skilled in the art or as previously described, affords a compound of formula (XXVII), where R³ is as defined in claim 1.

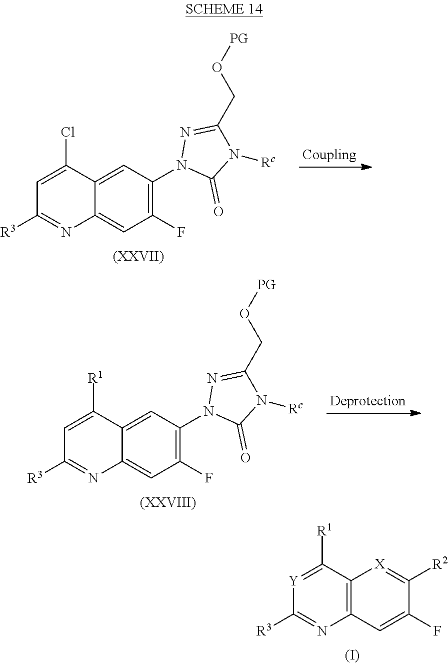

According to SCHEME 14, a compound of formula (XXVII), where R³ is as defined in claim 1; is reacted in a metal mediated cross coupling reaction with 4,4,5,5-tetramethyl-2-(prop-1-en-2-yl)-1,3,2-dioxaborolane; employing methods known to one skilled in the art or previously described, to afford a compound of formula (XXVIII), where R¹ is C₂₋₄alkenyl. Deprotection of the PG benzyl on a compound of formula (XXVIII) is achieved according to procedures known to one skilled in the art, for example, employing BBr₃, BCl₃, and the like; in a suitable solvent such as dichloromethane (DCM), and the like; at temperatures ranging from −78 to 0° C.; to provide a compound of Formula (I), where X and Y are CH.

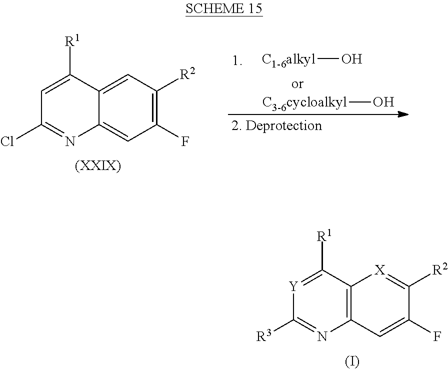

According to SCHEME 15, a compound of formula (XXIX) (which encompasses compounds of formulas (X), (Xa), and (Xb)) is reacted with a suitably substituted alkyl alcohol or cycloalkyl alcohol of formula C₁₋₆alkyl-OH or C₃₋₆cycloalkyl-OH; a suitable base such as NaH, K₂CO₃, Na₃CO₃, KHMDS, preferably NaH; at a temperature of about 50° C.; wherein when a compound of formula (XXIX) contains a benzyl protecting group on the R² moiety, subsequent cleavage of the benzyl protecting group is achieved according to procedures known to one skilled in the art or previously described to provide a compound of Formula (I), where X and Y are CH.

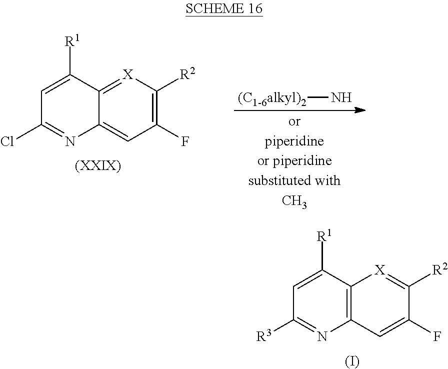

According to SCHEME 16, a compound of formula (XXIX) (which encompasses compounds of formulas (X), (Xa), and (Xb)) is reacted with a commercially available or synthetically accessible suitably substituted alkyl amine (C₁₋₆alkyl)₂-NH, piperidine, or methylpiperidine; with or without a base such as tBuONa; with our without a catalyst such as PEPPSI-iPr; neat or with a suitable solvent such as THF, dioxane, and the like; at a temperature of about 50° C. to 100° C.; for a period of 16 hours. Subsequent deprotection of benzyl protecting group on the R² moiety, is achieved according to procedures known to one skilled in the art or previously described to provide a compound of Formula (I), where X and Y are CH.

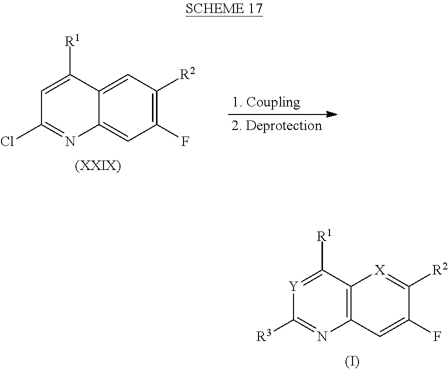

According to SCHEME 17, a compound of formula (XXIX) (which encompasses compounds of formulas (X), (Xa), and (Xb)) is reacted with a commercially available or synthetically accessible suitably substituted aryl, heteroaryl boronic ester or boronic acid; employing methods known to one skilled in the art, or as previously described to provide a compound of Formula (I). Subsequent deprotection of benzyl protecting group on the R² moiety, is achieved according to procedures known to one skilled in the art or previously described to provide a compound of Formula (I), where X and Y are CH. Oxidation of a compound Formula (I), where X and Y are CH is achieved employing mCPBA, in a suitable solvent such as CHCl₃, at temperatures ranging from rt to 70° C., to provide an N-oxide compound of Formula (I).

A compound of Formula (I), where R³ is 2-methoxy-3,5-dimethylpyridin-4-yl moiety is reacted with concentrated HCl, in dioxane, at 100° C., for 8 hours, to provide 2-hydroxy-3,5-dimethylpyridin-4-yl. Subsequent deprotection of benzyl protecting group on the R² moiety, is achieved according to procedures known to one skilled in the art or previously described to provide a compound of Formula (I), where X and Y are CH.

In an alternate method, a compound of formula (XXIX), is reacted with a heteroaryl compound such as 3,5-dimethyl-1H-pyrazole, and the like; a suitable base such as NaH, and the like; in a suitable solvent such as DMF; at temperatures ranging from 0° C. to 50° C.; for a period of 12-18 hrs; to afford a compound of Formula (I). Subsequent deprotection of benzyl protecting group on the R² moiety, is achieved according to procedures known to one skilled in the art or previously described to provide a compound of Formula (I), where X and Y are CH.

In an alternate method, a compound of formula (XXIX), is reacted with a suitably substituted aryl zinc reagent such as tetrahydropyran-4-ylzinc bromide, in a metal mediated coupling reaction; employing 2-dicyclohexylphosphino-2′,6′-bis(N,N-dimethylamino)biphenyl (Cphos); a palladium catalyst such as Pd(OAc)₂; in a suitable solvent such as DMA; at a temperature of about 35° C.; for a period of 16 hours. Subsequent deprotection of benzyl protecting group on the R² moiety, is achieved according to procedures known to one skilled in the art or previously described to provide a compound of Formula (I), where X and Y are CH.

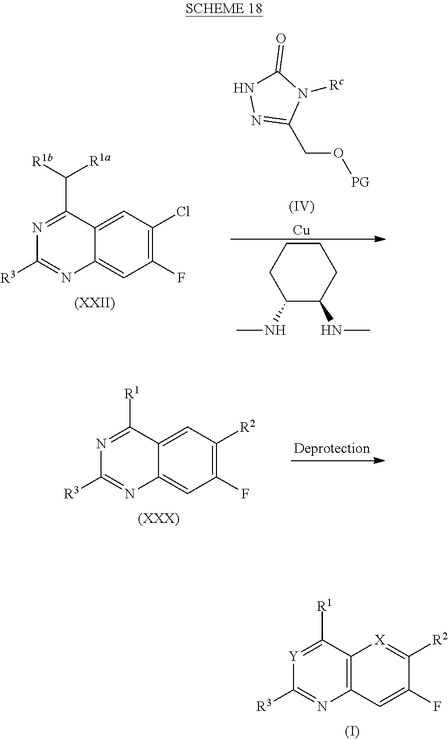

According to SCHEME 18, a compound of formula (XXII), is reacted with a commercially available or synthetically accessible compound of formula (IV) where R^{1a} and R^{1b} are CH₃, R^{c} is as defined in claim 1 and PG is benzyl; employing copper catalyzed arylation conditions previously described to provide a compound of formula (XXX). Subsequent cleavage of the benzyl protecting group on a compound of formula (XX) is achieved according to procedures known to one skilled in the art or previously described to provide a compound of Formula (I), where X is CH and Y is N.

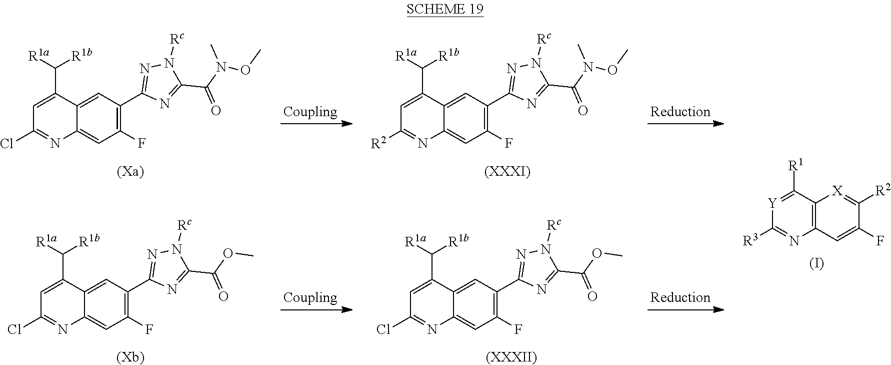

According to SCHEME 19, compounds of formula (Xa) and (Xb) are reacted in metal mediated cross coupling reaction with a commercially available or synthetically accessible suitably substituted aryl or heteroaryl boronic acid or boronic ester or cyclic boronate; employing conditions known to one skilled in the art or as previously described to provide compounds of (XXXI) and (XXXII). Compounds of formula (XXXI) or (XXXII) are reduced in either one or two steps employing a reducing agent such as LAH, NaBH₄, diisobutylaluminum hydride, and the like; in a suitable solvent such as THF, and the like, at temperatures ranging from −78° C. to 20°; to provide a compound of Formula (I), where X and Y are CH, and the R² moiety has a R^{b} substituent which is C₁₋₆alkyl substituted with OH.

Alternately, a compound of formula (XXXI), is reacted first with methylmagnesiun bromide, and subsequently reduced with a reducing agent such as NaBH₄, diisobutylaluminum hydride, and the like; in a suitable solvent such as THF, and the like, at temperatures ranging from −78° C. to 20°; to provide a compound of Formula (I), where X and Y are CH, and the R² moiety has a R substituent which is CH(OH)CH₃).

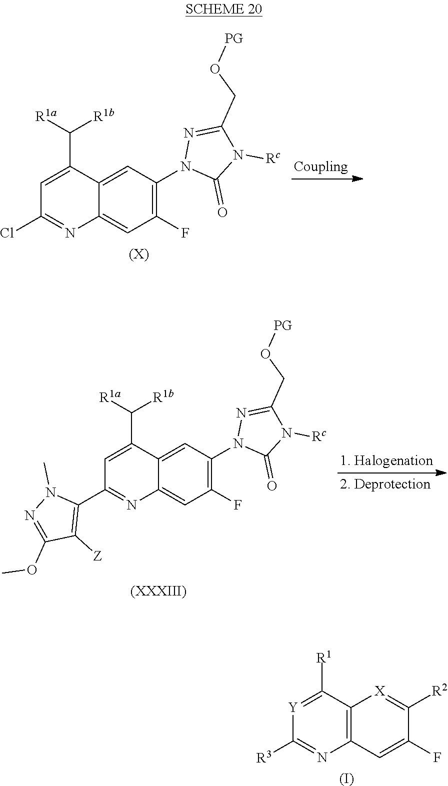

According to SCHEME 20, a compound of formula (X), is coupled with a suitably substituted commercially available or synthetically accessible heteroaryl boronic ester or boronic acid, employing methods known to one skilled in the art or previously described to provide a compound of formula (XXXIII), where Z is H. Halogenation of a compound of formula (XXXIII), is achieved employing 1-chloropyrrolidine-2,5-dione (NCS), in a suitable solvent such as ACN, and the like. Subsequent deprotection of benzyl protecting group on the R² moiety, is achieved according to procedures known to one skilled in the art or previously described to provide a compound of Formula (I), where X and Y are CH, and R³ is

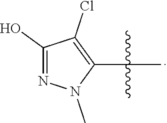 [SMILES: *c1c(Cl)c(O)nn1C]

A compound of Formula (I), where X and Y are CH, and R³ is

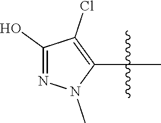 [SMILES: *c1c(Cl)c(O)nn1C]

alkylated employing conditions previously described to provide a compound of Formula (I) where R³ is

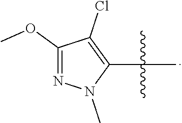 [SMILES: *c1c(Cl)c(OC)nn1C]

In a similar fashion, a compound of formula (X), is coupled with (3-(trifluoromethyl)-1H-pyrazol-4-yl)boronic acid, employing methods previously described. Subsequent chlorination and deprotection of the benzyl moiety is achieved employing methods previously described to afford a compound of Formula (I), where R3 is

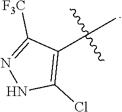 [SMILES: *c1c(C(F)(F)F)n[nH]c1Cl]

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form.

Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

The following specific examples are provided to further illustrate the invention and various preferred embodiments.

### Examples

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on either:

METHOD A. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (with 0.225% FA) over 10 min and then hold at 100% ACN for 2 min, at a flow rate of 25 mL/min.
or
METHOD B. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.1% TFA) over 10 min and then hold at 100% ACN for 2 min, at a flow rate of 25 mL/min.
or
METHOD C. A Gilson GX-281 semi-prep-HPLC with Phenomenex Synergi C18 (10 μm, 150×25 mm), or Boston Green ODS C18 (5 μm, 150×30 mm), and mobile phase of 5-99% ACN in water (0.05% HCl) over 10 min and then hold at 100% ACN for 2 min, at a flow rate of 25 mL/min.
or
METHOD D. a Gilson GX-281 semi-prep-HPLC with Phenomenex Gemini C18 (10 μm, 150×25 mm), AD (10 μm, 250 mm×30 mm), or Waters XBridge C18 column (5 μm, 150×30 mm), mobile phase of 0-99% ACN in water (with 0.05% ammonia hydroxide v/v) over 10 min and then hold at 100% ACN for 2 min, at a flow rate of 25 mL/min.
or
METHOD E. a Gilson GX-281 semi-prep-HPLC with Phenomenex Gemini C18 (10 μm, 150×25 mm), or Waters XBridge C18 column (5 μm, 150×30 mm), mobile phase of 5-99% ACN in water (10 mM NH₄HCO₃) over 10 min and then hold at 100% ACN for 2 min, at a flow rate of 25 mL/min.
or
METHOD F. Teledyne ISCO ACCQPrep HP150 semi-prep-HPLC with Phenomenex Gemini-NX C18 (5 μm, 150×30 mm), mobile phase of 10-100% ACN in water (10 mM NH₄OH) over 10 min and then hold at 100% ACN for 2 min, at a flow rate of 30 mL/min.

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature

Mass spectra (MS) were obtained on a SHIMADZU LCMS-2020 MSD or Agilent 1200\G611OA MSD using electrospray ionization (ESI) in positive mode unless otherwise indicated. Calculated (calcd.) mass corresponds to the exact mass.

Nuclear magnetic resonance (NMR) spectra were obtained on Bruker model AVIII 400 spectrometers. Definitions for multiplicity are as follows: s=singlet, d=doublet, t=triplet, q=quartet, td=triplet of doublets, sept=septet; m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 17.1 (CambridgeSoft Corp., Cambridge, Mass.) or OEMetaChem V1.4.0.4 (Open Eye).

Compounds designated as R* or S* are enantiopure compounds where the absolute configuration was not determined.

### EXAMPLES

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

### Intermediate 1: 3-((Benzyloxy)methyl)-4-ethyl-1H-1,2,4-triazol-5 (4H)-one

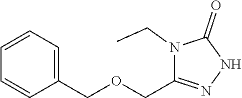 [SMILES: CCn1c(COCc2ccccc2)n[nH]c1=O]

Step A. 2-(Benzyloxy)acetohydrazide. To a solution of ethyl 2-(benzyloxy)acetate (55 g, 283.17 mmol) in EtOH (500 mL) was added NH₂NH₂.H₂O (28.3 g, 566 mmol, 27.5 mL). The mixture was heated reflux at 78° C. stirred for 6 hr. The reaction mixture was concentrated under reduced pressure to get the title compound (52 g, crude) was obtained as a colorless oil, which was used directly to next step without further purification.

Step B. 3-((Benzyloxy)methyl)-4-ethyl-1H-1,2,4-triazol-5 (4H)-one. To a solution of 2-(benzyloxy)acetohydrazide (52 g, 288 mmol) in H₂O (500 mL) was added dropwise isocyanatoethane (25.1 g, 346 mmol, 27.9 mL) at 0° C. After addition, the mixture was stirred at 25° C. for 12 hr. To the reaction mixture was added H₂O (20 mL), and an aqueous solution of NaOH (57.7 g, 1.44 mol, in 120 mL of H₂O). The reaction mixture was stirred at 95° C. for 12 hr. The reaction mixture was quenched with HCl (12M) at 0° C. and adjusted to pH to 6. The resulting solid was filtered and dried under reduced pressure to get the title product (61 g, 261 mmol, 91% yield) as a white solid. ¹H NMR (400 MHz, CDCl₃) S=9.23-9.09 (m, 1H), 7.41-7.31 (m, 5H), 4.58-4.53 (m, 2H), 4.45-4.42 (m, 2H), 3.82-3.75 (m, 2H), 1.33-1.29 (m, 3H).

### Intermediate 2: 3-((Benzyloxy)methyl)-1-(2-chloro-7-fluoro-4-isopropylquinolin-6-yl)-4-ethyl-1H-1,2,4-triazol-5 (4H)-one

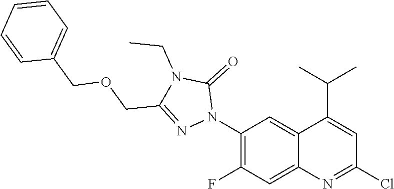 [SMILES: CCn1c(COCc2ccccc2)nn(-c2cc3c(C(C)C)cc(Cl)nc3cc2F)c1=O]

Step A. N-(4-Bromo-3-fluorophenyl)-4-methyl-3-oxopentanamide. A mixture of methyl 4-methyl-3-oxopentanoate (10 g, 69.36 mmol), 4-bromo-3-fluoroaniline (14.5 g, 76.31 mmol), and Et₃N (1.8 g, 17.79 mmol) in toluene (70 mL) was heated to 70° C. The reaction mixture was stirred at 70° C. for 1 hour and then gradually heated to 110° C. and stirred at 110° C. overnight. After cooling to room temperature, the mixture was washed with 5% aq. HCl (100 mL) and water (100 mL×2). The organic layer was dried with anhydrous Na₂SO₄, filtered, and concentrated under reduced pressure. The residue was purified by flash column chromatography (SiO₂, petroleum ether/ethyl acetate=I/O to 9/1) to give the title compound (7.8 g, 21.29 mmol, 30.70% yield) as a brown solid. MS (ESI): mass calcd. for C₁₂H₁₃BrFNO₂, 301.0; m/z found, 303.8 [M+H]⁺. ¹H NMR (400 MHz, CDCl₃) δ=9.51 (br s, 1H), 7.67 (dd, J=2.3, 10.5 Hz, 1H), 7.48 (t, J=8.2 Hz, 1H), 7.13 (dd, J=1.5, 8.8 Hz, 1H), 3.63 (s, 2H), 2.75 (td, J=7.0, 13.9 Hz, 1H), 1.20 (d, J=6.8 Hz, 6H).

Step B. 6-Bromo-7-fluoro-4-isopropylquinolin-2-ol. A solution of N-(4-bromo-3-fluorophenyl)-4-methyl-3-oxopentanamide (3.7 g, 10.05 mmol) in conc. H₂SO₄ (19 mL) was stirred at 50° C. for 2 days. The mixture was cooled to room temperature and poured on a mixture of ice and sat. aq. Na₂CO₃ (700 mL). The mixture was filtered, and the filter cake was washed with H₂O (100 mL×2). The solid was purified by flash column chromatography (SiO₂, petroleum ether/ethyl acetate=I/O to 7/3) to give the title compound (1.75 g, 6.14 mmol, 61.05% yield) as a yellow solid. MS (ESI): mass calcd. for C₁₂H₁₁BrFNO, 283.0; m/z found, 285.8 [M+H]⁺. ¹H NMR (400 MHz, CDCl₃) δ=11.99 (br s, 1H), 7.97 (d, J=7.0 Hz, 1H), 7.20 (d, J=8.8 Hz, 1H), 6.61 (s, 1H), 3.33 (td, J=6.9, 13.6 Hz, 1H), 1.36 (d, J=6.8 Hz, 6H); ¹⁹F NMR (376 MHz, CDCl₃) δ=−103.06 (s, 1F).

Step C. 3-((Benzyloxy)methyl)-4-ethyl-1-(7-fluoro-2-hydroxy-4-isopropylquinolin-6-yl)-1H-1,2,4-triazol-5 (4H)-one. 3-((Benzyloxy)methyl)-4-ethyl-1H-1,2,4-triazol-5 (4H)-one (Intermediate 1, 295 mg, 1.27 mmol) and Cs₂CO₃ (618 mg, 1.90 mmol) was added slowly into the solution of 6-bromo-7-fluoro-4-isopropylquinolin-2-ol (300 mg, 1.05 mmol) in dioxane (8 mL) at room temperature under N₂. Then to the mixture was added CuI (100 mg, 525.1 μmol), KI (123 mg, 741 μmol) and trans-N,N-dimethylcyclohexane-1,2-diamine (90 mg, 632.7 μmol) under N₂. After addition, the reaction mixture was stirred at 115° C. for 16 hours. The mixture was diluted with H₂O (20 mL) and extracted with ethyl acetate (20 mL×3). The organic layer was dried over Na₂SO₄, filtered, and evaporated under reduced pressure. The crude product was purified by flash column chromatography (SiO₂, 0-80% ethyl acetate in petroleum ether) to give the title compound (360 mg, 719.8 μmol, 68.28% yield) as a white solid. MS (ESI): mass calcd. for C₂₄H₂₅FN₄O₃, 436.2; m/z found, 437.2 [M+H]⁺. ¹H NMR (400 MHz, CDCl₃) δ=7.93 (d, J=7.5 Hz, 1H), 7.37-7.32 (m, 5H), 7.17 (br d, J=10.4 Hz, 1H), 6.59 (s, 1H), 4.60 (s, 2H), 4.50 (s, 2H), 3.85 (m, J=7.4 Hz, 2H), 3.36-3.28 (m, 1H), 1.37-1.31 (m, 9H); ¹⁹F NMR (376 MHz, CDCl₃) δ=−116.37-−116.46 (m, 1F).

Step D. 3-((Benzyloxymethyl-1-(2-chloro-7-fluoro-4-isopropylquinolin-6-yl-4-ethyl-1H-1,2,4-triazol-5 (4H)-one. POCl₃ (3 mL) was added slowly into the solution of 3-((benzyloxy)methyl)-4-ethyl-1-(7-fluoro-2-hydroxy-4-isopropylquinolin-6-yl)-1H-1,2,4-triazol-5 (4H)-one (360 mg, 719.8 μmol) in toluene (5 mL) at room temperature. After addition, the mixture was stirred at 95° C. for 1 hour. The mixture was slowly added into H₂O (20 mL) and extracted with DCM (20 mL×3). The organic layer was dried over Na₂SO₄, filtered, and evaporated under reduced pressure. The crude product was purified by flash column chromatography (SiO₂, 0-80% ethyl acetate in petroleum ether) to give the title compound (280 mg, 408.35 μmol, 56.73% yield) as a white solid. MS (ESI): mass calcd. for C₂₄H₂₄ClFN₄O₂, 454.2; m/z found, 455.1 [M+H]⁺. ¹H NMR (400 MHz, CDCl₃) δ=8.31 (d, J=8.0 Hz, 1H), 7.84 (d, J=11.0 Hz, 1H), 7.45-7.33 (m, 5H), 7.31 (s, 1H), 4.65 (s, 2H), 4.60-4.54 (m, 2H), 3.91 (m, J=7.1 Hz, 2H), 3.67 (td, J=6.8, 13.7 Hz, 1H), 1.45-1.40 (m, 9H); ¹⁹F NMR (376 MHz, CDCl₃) δ=−116.37 (s, 1F).

### Intermediate 3: 3-Chloro-5-methyl-4-(4,4,5,5-tetramethyl-1,3,2-dioxaborolan-2-yl)-1H-pyrazole

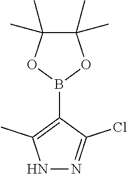 [SMILES: Cc1[nH]nc(Cl)c1B1OC(C)(C)C(C)(C)O1]

Step A. 4-Bromo-3-chloro-5-methyl-1H-pyrazole. 3-Chloro-5-methyl-1H-pyrazole (2.5 g, 21.45 mmol) was dissolved in DCM (50 mL), and then NBS (3.8 g, 21.45 mmol) was added. The reaction mixture was stirred at room temperature overnight. The reaction mixture was diluted with DCM (100 mL), washed with sat. aq. Na₂S₂O₃ (100 mL) and brine (100 mL). The organic layer was separated, dried over Na₂SO₄, filtered, and concentrated under reduced pressure. Purification by flash column chromatography (SiO₂, petroleum ether/DCM=I/O to 0/1, then DCM/MeOH=I/O to 20/1) afforded the title compound (3.9 g, 19.95 mmol, 93.03% yield) as a white solid. MS (ESI): mass calcd. for C₄H₄BrClN₂, 193.9; m/z found, 196.8 [M+H]⁺. ¹H NMR (400 MHz, CDCl₃) δ=11.49 (br s, 1H), 2.35 (s, 3H).

Step B. 3-Chloro-5-methyl-4-(4,4,5,5-tetramethyl-1,3,2-dioxaborolan-2-yl)-1H-pyrazole. [1,1′-Bis(diphenylphosphino)ferrocene]dichloropalladium(II) (Pd(dppf)Cl₂) (418 mg, 511.65 μmol) was added to 4-bromo-3-chloro-5-methyl-1H-pyrazole (1 g, 5.12 mmol), bis(pinacolato)diboron (2.6 g, 10.23 mmol) and AcOK (2 g, 20.47 mmol) in dioxane (13 mL) at room temperature under N₂. The mixture was stirred at 100° C. overnight under N₂ then cooled down to room temperature. The reaction mixture was filtered through a pad of Celite® and the solid was rinsed with 20 mL ethyl acetate. The filtrate was concentrated under reduced pressure. Purification by flash column chromatography (SiO₂, petroleum ether/DCM=I/O to 0/1, then DCM/MeOH=I/O to 20/1) afforded the title compound (500 mg, 991.98 μmol, 19.39% yield) as a white solid. MS (ESI): mass calcd. for C₁₀H₁₆BClN₂O₂, 242.1; m/z found, 243.0 [M+H]⁺. ¹H NMR (400 MHz, CDCl₃) δ=2.46 (s, 3H), 1.34 (s, 12H).

### Intermediate 4: 5-Methyl-4-(4,4,5,5-tetramethyl-1,3,2-dioxaborolan-2-yl)-3-(trifluoromethyl)-pH-pyrazole

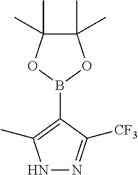 [SMILES: Cc1[nH]nc(C(F)(F)F)c1B1OC(C)(C)C(C)(C)O1]

Bis(pinacolato)diboron (646 mg, 2.55 mmol), AcOK (416 mg, 4.24 mmol) and Pd(dppf)Cl₂ (69 mg, 84.84 μmol) was added to 4-bromo-5-methyl-3-(trifluoromethyl)-1H-pyrazole (200 mg, 848.4 μmol) in dioxane (4 mL) at room temperature under N₂. The mixture was stirred at 100° C. overnight under N₂ then cooled down to room temperature. The reaction mixture was filtered over Celite®, and the solid was rinsed with ethyl acetate (10 mL). The filtrate was concentrated under reduced pressure. Purification by flash column chromatography (SiO₂, petroleum ether/DCM=I/O to 0/1) to give the title compound (45 mg, 129.68 μmol, 15.29% yield) as a yellow solid. MS (ESI): mass calcd. for C₁₁H₁₆BF₃N₂O₂, 276.1; m/z found, 277.1 [M+H]⁺. ¹H NMR (400 MHz, CDCl₃) δ=2.49 (s, 3H), 1.32 (s, 12H); ¹⁹F NMR (376 MHz, CDCl₃) δ=−61.83 (s, 1F).

### Intermediate 5: 2-Methoxy-3,5-dimethyl-4-(4,4,5,5-tetramethyl-1,3,2-dioxaborolan-2-yl)pyridine

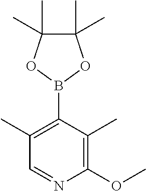

Step A. 4-Bromo-2-methoxy-3,5-dimethylpyridine. To a mixture of t-butyl nitrite (0.8 mL, 6.57 mmol) in MeCN (10 mL) was added CuBr (1.4 g, 9.86 mmol). The mixture was stirred at 70° C. for 10 minutes. Then a mixture of 2-methoxy-3,5-dimethylpyridin-4-amine (500 mg, 3.29 mmol) in MeCN (5 mL) was added drop wise to the reaction mixture. After stirring at 70° C. for one hour, the reaction mixture was cooled to room temperature. The reaction mixture was concentrated under reduced pressure. Ethyl acetate (20 mL) and sat. aq. NaHCO₃ (10 mL) were added to the residue, and the mixture was stirred at room temperature for 30 minutes. The mixture was filtered, and the filtrate was extracted with ethyl acetate (20 mL×2). The combined organic layers were dried over Na₂SO₄, filtered, and concentrated under reduced pressure. The crude product was purified by flash column chromatography (SiO₂, gradient elution: 0-2% ethyl acetate in petroleum ether) to give the title compound (480 mg, 2.19 mmol, 66.61% yield) as a colorless oil. MS (ESI): mass calcd. for C₈H₁₀BrNO, 215.0; m/z found, 216.0 [M+H]⁺. 15 ¹H NMR (400 MHz, CDCl₃) δ=7.80 (s, 1H), 3.92 (s, 3H), 2.30 (s, 3H), 2.29 (s, 3H).

Step B. 2-Methoxy-3,5-dimethyl-4-(4,4,5,5-tetramethyl-1,3,2-dioxaborolan-2-yl)pyridine. To a mixture of 4-bromo-2-methoxy-3,5-dimethylpyridine (430 mg, 1.96 mmol) in dioxane (10 mL) was added bis(pinacolato)diboron (995 mg, 3.92 mmol) and potassium acetate (769 mg, 7.84 mmol), followed by the addition of [1,1′-bis(diphenylphosphino)ferrocene]dichloropalladium(II) (Pd(dppf)Cl₂) (143 mg, 196.05 μmol) under N₂. The resulting mixture was charged with N₂ and stirred at 100° C. under N₂ overnight. The mixture was cooled to room temperature. The mixture was filtered, the filtrate was concentrated under reduced pressure. The residue was purified by flash column chromatography (SiO₂, gradient elution: 0-6% ethyl acetate in petroleum ether) to give the title compound (290 mg, 1.10 mmol, 56.21% yield) as a white powder. MS (ESI): mass calcd. for C₁₄H₂₂BNO₃, 263.2; m/z found, 264.2 [M+H]⁺. ¹H NMR (400 MHz, CDCl₃) δ=7.73 (s, 1H), 3.88 (s, 3H), 2.21 (d, J=12.6 Hz, 6H), 1.37 (s, 12H).

### Intermediate 6: 3-Chloro-2-methoxy-5-methyl-4-(4,4,5,5-tetramethyl-1,3,2-dioxaborolan-2-yl)pyridine

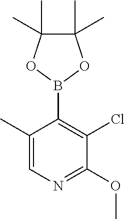

Step A. 4-Bromo-3-chloro-2-methoxy-5-methylpyridine. A mixture of CuBr (1.25 g, 8.69 mmol) and t-butyl nitrite (597 mg, 5.79 mmol) was stirred in MeCN (6 mL) at 70° C. for 10 minutes. A mixture of 3-chloro-2-methoxy-5-methylpyridin-4-amine (500 mg, 2.90 mmol) in MeCN (6 mL) was added drop wise to the reaction mixture and the mixture was stirred at 70° C. overnight. The reaction mixture was cooled to room temperature and concentrated under reduced pressure. Ethyl acetate (30 mL) and sat. aq. NaHCO₃ solution (25 mL) was added, and the reaction mixture was stirred at room temperature for 30 minutes. The reaction mixture was filtered, and the filtrate was extracted with ethyl acetate (25 mL×3). The combined organic phase was dried with anhydrous Na₂SO₄, filtered, and concentrated under reduced pressure. The crude product was purified by flash column chromatography (SiO₂, 0-30% ethyl acetate in petroleum ether) to give the title compound (390 mg, 1.57 mmol, 54.05% yield) as a yellow oil. MS (ESI): mass calcd. for C₇H₇BrClNO, 234.9; m/z found, 236.0 [M+H]⁺. ¹H NMR (400 MHz, CDCl₃) δ=7.84 (s, 1H), 4.01-3.92 (m, 3H), 2.31 (s, 3H).

Step B. 3-Chloro-2-methoxy-5-methyl-4-(4,4,5,5-tetramethyl-1,3,2-dioxaborolan-2-yl)pyridine. To a mixture of 4-bromo-3-chloro-2-methoxy-5-methylpyridine (390 mg, 1.57 mmol) in dioxane (12 mL) was added bis(pinacolato)diboron (596 mg, 2.35 mmol) and potassium acetate (615 mg, 6.26 mmol), [1,1′-bis(diphenylphosphino)ferrocene]dichloropalladium(II), complex with dichloromethane (Pd(dppf)Cl₂.DCM) (128 mg, 156.6 μmol) under N₂. The resulting reaction mixture was charged with N₂ and stirred at 100° C. for 16 hours. The reaction mixture was filtered, and the filter cake was washed with ethyl acetate (30 mL). The filtrate was evaporated under reduced pressure. The crude product was purified by flash column chromatography (SiO₂, gradient elution: 0-40% ethyl acetate in petroleum ether) to give the title compound (270 mg, 952.18 μmol, 60.82% yield) as a white solid. MS (ESI): mass calcd. for C₁₃H₁₉BClNO₃, 283.1; m/z found, 284.1 [M+H]⁺. ¹H NMR (400 MHz, CDCl₃) δ=7.74 (s, 1H), 3.90 (s, 3H), 2.18 (s, 3H), 1.34 (s, 12H).

### Intermediate 7: 3-Bromo-N-methoxy-N,1-dimethyl-1H-1,2,4-triazole-5-carboxamide

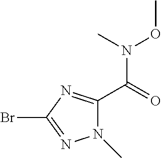

N,O-Dimethylhydroxylamine hydrochloride (399 mg, 4.1 mmol) was added to a solution of bis(trimethylaluminum)-1,4-diazabicyclo[2.2.2]octane adduct (838.9 mg, 3.3 mmol) in THF and warmed to 40° C. for 30 min. After 30 min, methyl 3-bromo-1-methyl-1H-1,2,4-triazole-5-carboxylate (600 mg, 2.3 mmol) was added and the reaction mixture was heated to 70° C. for 18 hours. The reaction mixture was cooled to room temperature and quenched, slowly, with 2N HCl. The reaction mixture was extracted with ethyl acetate (3×) and the combined organic layers were washed with brine, dried over Na₂SO₄, filtered, and concentrated under reduced pressure. The title compound (605 mg crude) was obtained as a pale yellow solid, which was used without further purification. MS (ESI): mass calcd. for C₆H₉BrN₄O₂, 247.99; m/z found, 248.9 [M+H]⁺.

### Intermediate 8: 3-(2-Chloro-7-fluoro-4-isopropylquinolin-6-yl)-N-methoxy-N,1-dimethyl-1H-1,2,4-triazole-5-carboxamide

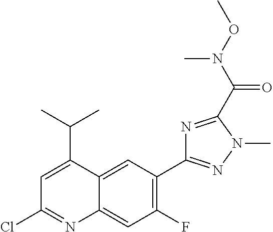 [SMILES: CON(C)C(=O)c1nc(-c2cc3c(C(C)C)cc(Cl)nc3cc2F)nn1C]

Step A: (7-Fluoro-2-hydroxy-4-isopropylquinolin-6-yl)boronic acid, Pd(dppf)Cl₂.DCM (4.91 g, 6.01 mmol) was added to a mixture of 6-bromo-7-fluoro-4-isopropylquinolin-2-ol (Intermediate 2, product from Step B, 20 g, 60.12 mmol), bis(pinacolato)diboron (22.90 g, 90.18 mmol) and KOAc (17.70 g, 180.35 mmol) in dioxane (500 mL) at room temperature under N₂. The reaction mixture was stirred at 85° C. overnight under N₂. The reaction mixture was evaporated under reduced pressure. H₂O (500 mL) was added to the residue, and the mixture was extracted with DCM (2×). The organic layer was separated, washed with brine, dried over Na₂SO₄, and evaporated under reduced pressure. The crude material was purified by flash column chromatography (SiO₂, 0-100% ethyl acetate in petroleum ether) to give an impure mixture of the desired compound (45 g) as a yellow solid. The mixture was further purified by preparative reversed phase HPLC (Stationary phase: YMC Exphere C18, 10 μm, 250×50 mm; Mobile phase: water (0.05% NH₃H₂O+10 mM NH₄HCO₃) (A) —MeCN (B), gradient elution: 5-45% B in A over 20 min, flow rate: 120 mL/min) to give the title compound (3.3 g, 12.52 mmol, 20.83% yield, and second fraction 1.2 g, 4.81 mmol, 8.00% yield) as off-white powder. MS (ESI): mass calcd. for C₁₂H₁₃BFNO₃, 249.1; m/z found, 250.0 [M+H]⁺. ¹H NMR (400 MHz, DMSO-d₆) δ=11.66 (s, 1H), 8.18 (s, 2H), 8.08 (d, J=6.4 Hz, 1H), 6.96 (d, J=10.4 Hz, 1H), 6.30 (s, 1H), 3.43 (quin, J=6.7 Hz, 1H), 1.26 (d, J=6.8 Hz, 6H); ¹⁹F NMR (376 MHz, DMSO-d₆) δ=−101.10-101.15 (m, 1F).

Step B: 3-(7-Fluoro-2-hydroxy-4-isopropylquinolin-6-yl)-N-methoxy-N,1-dimethyl-1H-1,2,4-triazole-5-carboxamide. A mixture of 3-bromo-N-methoxy-N,1-dimethyl-1H-1,2,4-triazole-5-carboxamide (Intermediate 7, 531.7 mg, 2.14 mmol), (7-fluoro-2-hydroxy-4-isopropylquinolin-6-yl)boronic acid (483.3 mg, 1.9 mmol), XPhos Pd G3 (164.3 mg, 0.19 mmol), cesium carbonate (1896.8 mg, 5.8 mmol) in dioxane/water (5:1) were combined and the reaction vessel was purged with argon, and then heated to 80° C. for 1 hour. The reaction mixture was cooled to room temperature and subsequently diluted with ethyl acetate and water. The organic layer was extracted (3×) with ethyl acetate and the combined organics were washed with brine, dried over Na₂SO₄, filtered, and concentrated under reduced pressure. The title compound (822 mg crude) was obtained as a white solid and was used in the next step without further purification. MS (ESI): mass calcd. for C₁₈H₂₀FN₅O₃, 373.16; m/z found, 374.1 [M+H]⁺.

Step C: 3-(2-Chloro-7-fluoro-4-isopropylquinolin-6-yl)-N-methoxy-N,1-dimethyl-1H-1,2,4-triazole-5-carboxamide. To a solution of 3-(7-fluoro-2-hydroxy-4-isopropylquinolin-6-yl)-N-methoxy-N,1-dimethyl-1H-1,2,4-triazole-5-carboxamide (735 mg, 1.9 mmol) in toluene was added POCl₃ (4.0 mL, 1.645 g/mL, 42.9 mmol). The reaction mixture was stirred at 95° C. for 1 hour. The reaction mixture was cooled to room temperature, quenched with H₂O and diluted with DCM. The reaction mixture was transferred to a separatory funnel, extracted with DCM (2×) and ethyl acetate (1×). The combined organic layers were washed with brine, dried over Na₂SO₄, filtered, and concentrated under reduced pressure. Purification (FCC, SiO₂, 0-80% ethyl acetate in heptane) afforded the title compound (442.1 mg, 1.13 mmol, 57% yield over 2 steps) as a pale yellow solid. MS (ESI): mass calcd. for C₁₈H₁₉ClFN₅O₂, 391.12; m/z found, 374.1 [M+H]⁺. ¹H NMR (400 MHz, CDCl₃) δ=8.79 (br s, 1H), 7.78 (d, J=11.25 Hz, 1H), 7.28 (s, 1H), 4.15 (br s, 3H), 3.95 (s, 3H), 3.65-3.91 (m, 2H), 3.42 (br s, 2H), 1.43 (d, J=6.85 Hz, 6H).

### Intermediate 9: Methyl 4-(2-chloro-7-fluoro-4-isopropylquinolin-6-yl)-1-methyl-1H-imidazole-2-carboxylate

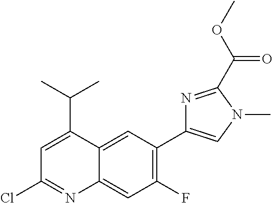 [SMILES: COC(=O)c1nc(-c2cc3c(C(C)C)cc(Cl)nc3cc2F)cn1C]

Step A: Methyl 4-(7-fluoro-2-hydroxy-4-isopropylquinolin-6-yl)-1-methyl-1H-imidazole-2-carboxylate. A solution of methyl 4-bromo-1-methyl-1H-imidazole-2-carboxylate (725.6 mg, 3.3 mmol), (7-fluoro-2-hydroxy-4-isopropylquinolin-6-yl)boronic acid (750 mg, 3.0 mmol), XPhos Pd G3 (254.9 mg, 0.301 mmol), cesium carbonate (2943.6 mg, 9.03 mmol) in 1,4-dioxane/water (5:1), was purged with argon, then heated to 80° C. After 5 minutes, solids crash out of reaction mixture. The reaction mixture was cooled to room temperature, diluted with water, and filtered. The resulting solids were washed with ethyl acetate and dried. The title compound (981 mg crude) was obtained as a white solid and was used in the next step without further purification. MS (ESI): mass calcd. for C₁₈H₁₈FN₃O₃, 343.13; m/z found, 344.1 [M+H]⁺.

Step B: Methyl 4-(2-chloro-7-fluoro-4-isopropylquinolin-6-yl)-1-methyl-1H-imidazole-2-carboxylate. To a solution of methyl 4-(7-fluoro-2-hydroxy-4-isopropylquinolin-6-yl)-1-methyl-1H-imidazole-2-carboxylate (980.6 mg, 2.9 mmol) in toluene (9.6 mL) was added POCl₃ (7.7 mL, 1.645 g/mL, 82.8 mmol). The reaction mixture was heated to 85° C. for 1 hour. The reaction mixture was cooled to room temperature, quenched with H₂O and diluted with DCM. The reaction mixture was extracted with DCM (2×) and ethyl acetate (1×). The combined organic layers were washed with brine, dried over Na₂SO₄, filtered, and concentrated under reduced pressure. Purification (FCC, SiO₂, 0-80% ethyl acetate in heptane) afforded the title compound (439 mg, 1.21 mmol, 43% yield over 2 steps) as a yellow solid. MS (ESI): mass calcd. for C₁₈H₁₇ClFN₃O₂, 361.10; m/z found, 362.1 [M+H]⁺. ¹H NMR (400 MHz, DMSO-d₆) δ=8.76 (d, J=8.31 Hz, 1H), 8.05 (d, J=4.40 Hz, 1H), 7.87 (d, J=12.23 Hz, 1H), 7.47 (s, 1H), 4.02-4.06 (m, 3H), 3.89-3.92 (m, 3H), 3.78 (quin, J=6.85 Hz, 1H), 1.39 (d, J=6.85 Hz, 6H).

### Intermediate 10: 2-Methoxy-4-methyl-3-(4,4,5,5-tetramethyl-1,3,2-dioxaborolan-2-yl)pyridine

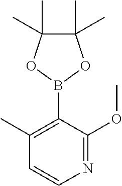

Bis(pinacolato)diboron (283 mg, 1.11 mmol) and KOAc (219 mg, 2.23 mmol) was added into a solution of 3-bromo-2-methoxy-4-methylpyridine (150 mg, 742.39 μmol) in dioxane (5 mL) at room temperature under N₂. To the reaction mixture was added Pd(dppf)Cl₂ (54 mg, 73.80 μmol) at room temperature under N₂. The reaction mixture was stirred at 85° C. overnight under N₂. Saturated aq. NH₄Cl (5 mL) was added into the reaction mixture and the reaction mixture was extracted with DCM. The organic layer was separated and washed with brine, dried over Na₂SO₄, and evaporated under reduced pressure. Purification (FCC, SiO₂, gradient elution: 0-100% ethyl acetate in petroleum ether) afforded the title compound (90 mg, 281.54 μmol, 37.92% yield) as a yellow solid. MS (ESI): mass calcd. for C₁₃H₂₀BNO₃, 249.2; m/z found, 250.1 [M+H]⁺. ¹H NMR (400 MHz, CDCl₃) δ=8.03 (d, J=5.2 Hz, 1H), 6.68 (d, J=5.2 Hz, 1H), 3.92 (s, 3H), 2.35 (s, 3H), 1.41 (s, 12H).

### Intermediate 11: 3-Methyl-4-(4,4,5,5-tetramethyl-1,3,2-dioxaborolan-2-yl)pyridin-2-ol

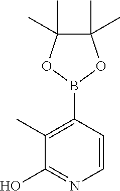

To a mixture of 4-bromo-3-methylpyridin-2-ol (300 mg, 1.60 mmol) in dioxane (10 mL) was added bis(pinacolato)diboron (1.22 g, 4.79 mmol) and KOAc (626.4 mg, 6.38 mmol). Pd(dppf)Cl₂ (116.7 mg, 159.6 mmol) was added to the reaction mixture and the reaction mixture was stirred under N₂. The resulting reaction mixture was charged with N₂ and stirred at 100° C. overnight then cooled down to room temperature. The reaction mixture was filtered, and the filtrate was concentrated under reduced pressure. The residue was purified by flash column chromatography (SiO₂, gradient elution: 0-80% ethyl acetate in petroleum ether) to give the title compound (210 mg, 392.5 μmol, 25% yield) as a light yellow solid. MS (ESI): mass calcd. for C₁₂H₁₈BNO₃, 235.1; m/z found, 236.2 [M+H]⁺.

### Intermediate 12: 2-(Benzyloxy)-5-methyl-4-(4,4,5,5-tetramethyl-1,3,2-dioxaborolan-2-yl)pyridine

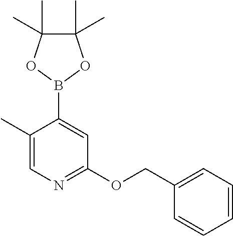 [SMILES: Cc1cnc(OCc2ccccc2)cc1B1OC(C)(C)C(C)(C)O1]

Step A. 2-(Benzyloxy)-4-iodo-5-methylpyridine. NaH (60% purity, 675 mg, 16.9 mmol) was added to a solution of phenylmethanol (2.28 g, 21.1 mmol) in THF (20 mL) at 0° C. under N₂. The reaction mixture was stirred at 0° C. for 1 hour, then 2-fluoro-4-iodo-5-methylpyridine (1 g, 4.2 mmol) was added and the reaction mixture stirred at room temperature for 2 hours. The reaction mixture was diluted with DCM (200 mL) and washed with brine (200 mL×3). The organic layer was dried with Na₂SO₄, filtered, and concentrated under reduced pressure. The crude product was purified by flash column chromatography (SiO₂, gradient elution: 0-10% ethyl acetate in petroleum ether), then further purified by preparative reversed phase HPLC (Stationary phase: Xtimate C18, 5 μm, 150×40 mm; Mobile phase: water (0.05% NH₃H₂O) (A) —MeCN (B), gradient elution: 80-100% B in A over 9 min, flow rate: 60 mL/min) to give the title compound (1.08 g, 3.3 mmol, 79% yield) as a colorless oil. MS (ESI): mass calcd. for C₁₃H₁₂INO, 325.0; m/z found, 325.9 [M+H]⁺. ¹H NMR (400 MHz, CDCl₃) δ=7.93 (s, 1H), 7.48-7.28 (m, 6H), 5.33 (s, 2H), 2.32 (s, 3H).

### Step B. 2-(Benzyloxy)-5-methyl-4-(4,4,5,5-tetramethyl-1,3,2-dioxaborolan-2-yl)pyridine

Bis(pinacolato)diboron (2.30 g, 9.04 mmol), AcOK (1.18 g, 12.06 mmol) and Pd(dppf)Cl₂ DCM (246 mg, 301.41 μmol) were added to a solution of 2-(benzyloxy)-4-iodo-5-methylpyridine (980 mg, 3.01 mmol) in dioxane (10 mL) at room temperature under N₂. The reaction mixture was stirred at 100° C. for 16 hours then cooled down to room temperature. The reaction mixture was filtered through a pad of Celite® and the solid was rinsed with DCM (50 mL). The filtrate was concentrated under reduced pressure and purified by flash column chromatography (SiO₂, gradient elution: 0-20% ethyl acetate in petroleum ether), then further purified by preparative reversed phase HPLC (Stationary phase: Xtimate C18, 5 μm, 150×40 mm; Mobile phase: H₂O (10 mM NH₄HCO₃) (A) —MeCN (B), gradient elution: 75-95% B in A over 8 min, flow rate: 60 mL/min) to give the title compound (220 mg, 613.8 μmol, 20% yield) as a green solid. MS (ESI): mass calcd. for C₁₉H₂₄BNO₃, 325.2; m/z found, 243.9 [M+H]⁺. ¹H NMR (400 MHz, CDCl₃) δ=7.99 (s, 1H), 7.49-7.33 (m, 5H), 7.18 (s, 1H), 5.36 (s, 2H), 2.40 (s, 3H), 1.36 (s, 12H).

### Intermediate 13: 3-Methoxy-1-methyl-5-(4,4,5,5-tetramethyl-1,3,2-dioxaborolan-2-yl)-1H-pyrazole

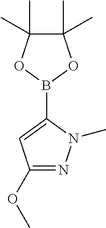

Step A. 3-Methoxy-1-methyl-1H-pyrazole. To a suspension of 1-methyl-1H-pyrazol-3-ol (2 g, 20.39 mmol) and K₂CO₃ (5.64 g, 40.77 mmol) in MeCN (20 mL) was added iodomethane (5.37 g, 37.83 mmol). The reaction mixture was stirred at room temperature for 2 hours. The reaction mixture was filtered and diluted with ethyl acetate (100 mL) and water (50 mL). The organic layer was separated, washed with brine (15 mL), dried over Na₂SO₄, filtered, and concentrated under reduced pressure. The residue was purified by flash column chromatography (SiO₂, gradient elution: 0-100% ethyl acetate in petroleum ether) to give the title compound (700 mg, 6.24 mmol, 91% yield) as a yellow oil. ¹H NMR (400 MHz, DMSO-d₆) δ=6.72 (d, J=1.4 Hz, 1H), 6.45 (d, J=1.4 Hz, 1H), 3.89 (s, 3H), 3.35-3.34 (m, 3H).

Step B. 3-Methoxy-1-methyl-5-(4,4,5,5-tetramethyl-1,3,2-dioxaborolan-2-yl)-1H-pyrazole. A flask was purged with N₂ and hexane (20 mL) was added. The solvent was bubbled with N₂ for 5 minutes and (1,5-cyclooctadienexmethoxy)iridium(I) dimer (70 mg, 105.60 μmol), 4,4′-di-tert-butyl-2,2′-bipyridine (56 mg, 208.6 μmol) and 4,4,5,5-tetramethyl-1,3,2-dioxaborolane (493 mg, 3.85 mmol) were added. The solution turned rapidly to deep red. After 15 minutes, 3-methoxy-1-methyl-1H-pyrazole (400 mg, 3.50 mmol) was added in one portion and the reaction mixture was stirred at room temperature for 16 hours under N₂. The solvent was removed under reduced pressure to give the crude product. The residue was purified by flash column chromatography (SiO₂, gradient elution: 0-100% ethyl acetate in petroleum ether) to give the title compound (450 mg, 1.89 mmol, 54% yield) as a yellow oil. MS (ESI): mass calcd. for C₁₁H₁₉BN₂O₃, 238.1; m/z found, 239.0 [M+H]⁺. ¹H NMR (400 MHz, CDCl₃) δ=6.06 (s, 1H), 3.94 (s, 3H), 3.87 (s, 3H), 1.35 (s, 12H).

### Intermediate 14: 4,4,5,5-Tetramethyl-2-(o-methyl-d₃-phenyl)-1,3,2-dioxaborolane

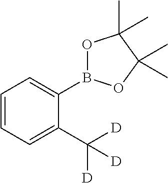 [SMILES: [2H]C([2H])([2H])c1ccccc1B1OC(C)(C)C(C)(C)O1]

Step A. 1-Bromo-2-(methoxymethoxy)benzene. To a solution of 2-bromophenol (4 g, 23.1 mmol) in DCM (40 mL) was added bromo(methoxy)methane (4.7 mL, 57.8 mmol) at 0° C. The reaction mixture was stirred at r.t. overnight. The reaction mixture was extracted with DCM (20 mL×3). The organic layers were separated, combined, dried with Na₂SO₄, filtered, and concentrated under reduced pressure. The residue was purified by flash column chromatography (SiO₂, gradient elution: 0-10% ethyl acetate in petroleum ether) give the title compound (4.4 g, 20.2 mmol, 87.7% yield) as yellow solid. ¹H NMR (400 MHz, CDCl₃) δ=7.55 (dd, J=1.5, 7.9 Hz, 1H), 7.32-7.24 (m, 1H), 7.16 (dd, J=1.3, 8.2 Hz, 1H), 6.90 (dt, J=1.5, 7.6 Hz, 1H), 5.26 (s, 2H), 3.54 (s, 3H) ppm.

Step B. 1-(Methoxymethoxy)-2-methyl-d₃-benzene. To a solution of n-BuLi (2.5 M in THF, 10.0 mL, 25.2 mmol) was added to a solution of 1-bromo-2-(methoxymethoxy)benzene (3.9 g, 17.9 mmol) in THF (15 mL) at −78° C. Immediately afterwards a solution of CD3I in THF (15 mL) was added to the reaction mixture. The reaction mixture was quenched with NH₄Cl (10 mL) at −78° C. The reaction mixture extracted with ethyl acetate (20 mL×3). The organic layer was separated, washed with brine (20 mL), dried with Na₂SO₄, filtered, and concentrated under reduced pressure. The residue was purified by flash column chromatography (SiO₂, gradient elution: 0-10% ethyl acetate in petroleum ether) give the title compound (2.5 g, 14.9 mmol, 83.1% yield) as colorless oil. ¹H NMR (400 MHz, CDCl₃) δ=7.30-7.28 (m, 1H), 7.27-7.24 (m, 1H), 7.19-7.15 (m, 1H), 7.04 (dt, J=1.1, 7.3 Hz, 1H), 5.33 (s, 2H), 3.63-3.61 (m, 3H) ppm.

Step C. o-Methyl-d₃-phenol. To a solution of 1-(methoxymethoxy)-2-methyl-d₃-benzene (2.5 g, 16.1 mol) in MeOH (20 mL) was added TsOH (306.4 mg, 1.61 mmol). The reaction mixture was stirred at 50° C. for 3 hours. The reaction mixture was extracted with ethyl acetate (20 mL×3). The organic layers were separated, combined, washed with brine (20 mL), dried with Na₂SO₄, filtered, and concentrated under reduced pressure. The residue was purified by flash column chromatography (SiO₂, gradient elution: 0-40% ethyl acetate in petroleum ether) give the title compound (1.6 g, 13.4 mmol, 83% yield) as colorless oil. ¹H NMR (400 MHz, CDCl₃) δ=7.15-7.11 (m, 1H), 7.10-7.06 (m, 1H), 6.86 (dt, J=1.0, 7.4 Hz, 1H), 6.78 (dd, J=0.9, 7.9 Hz, 1H), 4.79 (s 1H) ppm.

Step D. o-Methyl-d₃-phenyl trifluoromethanesulfonate. A solution of o-methyl-d₃-phenol (500 mg, 4.50 mmol) in pyridine (5 mL) was cooled in an ice-water bath. Tf2O (1.52 g, 5.40 mmol, 1.2 eq.) was added slowly to the reaction mixture. The reaction mixture was stirred at room temperature overnight. The reaction mixture was extracted with ethyl acetate (15 mL) and washed with H₂O (15 mL×2). The organic layer was washed with HCl (0.3 M in H₂O, 15 mL×4), sat. aq. NaHCO₃ solution (15 mL). The organic layer was dried with anhydrous Na₂SO₄, filtered, and concentrated under reduced pressure to give the title compound (690 mg, 2.8 mmol, 63% yield) as yellow oil. ¹H NMR (400 MHz, METHANOL-d₄) δ=7.43-7.19 (m, 4H); ¹⁹F NMR (376 MHz, METHANOL-d₄) δ=−75.89 (1F) ppm.

Step E. 4,4,5,5-Tetramethyl-2-(o-methyl-d₃-phenyl)-1,3,2-dioxaborolane. A solution of O-methyl-d₃-phenyl trifluoromethanesulfonate (300 mg, 1.23 mmol), 4,4,4′,4′,5,5,5′,5′-octamethyl-2,2′-bi(1,3,2-dioxaborolane) (626.5 mg, 2.47 mmol), Pd(dppf)Cl₂.DCM (201.5 mg, 246.94 μmol) and AcOK (302.6 mg, 3.08 mmol) in 1,4-dioxane (8 mL) was stirred at 90° C. under N₂ overnight. The reaction mixture was cooled to room temperature and ethyl acetate (20 mL) was added. The reaction mixture was filtered, and the filter cake was washed by ethyl acetate (10 mL). The filtrate was dried under vacuum to give the title compound (300 mg, 1.36 mmol, crude) as black oil.

### Intermediate 15: 3-methyl-4-(4,4,5,5-tetramethyl-1,3,2-dioxaborolan-2-yl)-5-(trifluoromethyl)-1H-pyrazole

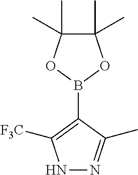 [SMILES: Cc1n[nH]c(C(F)(F)F)c1B1OC(C)(C)C(C)(C)O1]

Bis(pinacolato)diboron (646 mg, 2.55 mmol), AcOK (416 mg, 4.24 mmol) and Pd(dppf)Cl₂ (69 mg, 84.84 μmol) was added to 4-bromo-5-methyl-3-(trifluoromethyl)-1H-pyrazole (200 mg, 848.4 μmol) in dioxane (4 mL) at room temperature under N₂. The mixture was stirred at 100° C. overnight under N₂ then cooled down to room temperature.

The reaction mixture was filtered over Celite®, and the solid was rinsed with ethyl acetate (10 mL). The filtrate was concentrated under reduced pressure. Purification by flash column chromatography (SiO₂, petroleum ether/DCM=I/O to 0/1) to give the title compound (45 mg, 129.9 μmol, 15% yield) as a yellow solid. MS (ESI): mass calcd. for C₁₁H₁₆BF₃N₂O₂, 276.1; m/z found, 277.1 [M+H]⁺. ¹H NMR (400 MHz, CDCl₃) δ=2.49 (s, 3H), 1.32 (s, 12H); ¹⁹F NMR (376 MHz, CDCl₃) δ=−61.83 (s, 1F).

### Intermediate 16: 6-Chloro-7-fluoro-4-isopropyl-2-(o-tolyl)quinazoline

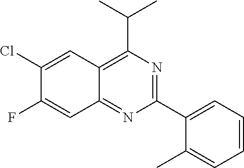 [SMILES: Cc1ccccc1-c1nc(C(C)C)c2cc(Cl)c(F)cc2n1]

Step A. N-(4-Chloro-3-fluorophenyl)-2-methylbenzamide. A mixture of 4-chloro-3-fluoroaniline (500 mg, 3.44 mmol), 2-methylbenzoyl chloride (531 mg, 3.44 mmol), Et₃N (71.6 μL, 0.515 mmol) in DCM (17.2 mL) was stirred at room temperature for 16 hours. The reaction was diluted with ethyl acetate and water and the layers were separated. The aqueous was extracted with ethyl acetate (3×) and the combined organic layers were washed with H₂O and brine. The dried organic layer was dried over Na₂SO₄, filtered, and concentrated under reduced pressure. Purification by flash column chromatography (SiO₂, 12G, 0 to 60% heptane/ethyl acetate) to give the title compound (837 mg, 3.17 mmol, 92% yield) as a white solid. MS (ESI): mass calcd. C₁₄H₁₁ClFNO, 263.05; m/z found 264.0 [M+H]⁺. ¹H NMR (400 MHz, CDCl₃) δ=ppm 2.50 (s, 3H) 7.18 (br d, J=8.31 Hz, 1H) 7.25-7.28 (m, 2H) 7.33-7.50 (m, 3H) 7.53 (br s, 1H) 7.75 (br d, J=10.76 Hz, 1H).

Step B. 6-Chloro-7-fluoro-4-isopropyl-2-(o-tolyl)quinazoline. Trifluoromethanesulfonic anhydride (1M in DCM) (1.21 mL, 1 M, 1.21 mmol) was added to N-(4-chloro-3-fluorophenyl)-2-methylbenzamide (290 mg, 1.1 mmol) and 2-chloropyridine (0.125 mL, 1.32 mmol) in dichloroethane (3.9 mL) at −78° C., over 1 minute. After 5 mins, reaction was warmed to 0° C. and isobutyronitrile (0.109 mL, 1.21 mmol) was added. The reaction mixture was warmed to room temperature for 5 mins and then heated under microwave irradiation at 140° C. for 20 mins. After reaction cools, 1 mL of 1N NaOH was slowly added. Reaction was diluted with DCM and transferred to a separatory funnel and the layers were separated. The organic layer was washed with brine, dried over Na₂SO₄, and concentrated under reduced pressure. Purification by flash column chromatography (SiO₂ 12G 0-40% heptane/ethyl acetate) yielded the title compound (235 mg, 0.747 mmol, yield 67.882%) as a yellow solid. MS (ESI): mass calcd. C₁₈H₁₆ClFN₂, 314.10; m/z found 315.0 [M+H]⁺. ¹H NMR (400 MHz, CDCl₃) δ=ppm 1.46-1.47 (m, 3H) 1.48-1.49 (m, 3H) 2.62-2.68 (m, 3H) 3.86 (dt, J=13.21, 6.60 Hz, 1H) 7.32-7.39 (m, 3H) 7.76-7.82 (m, 1H) 7.99-8.04 (m, 1H) 8.26 (d, J=7.83 Hz, 1H).

### Intermediate 17: 6-Bromo-7-fluoro-4-isopropyl-2-(3-methyl-5-(trifluoromethyl)-1H-pyrazol-4-yl)quinazoline

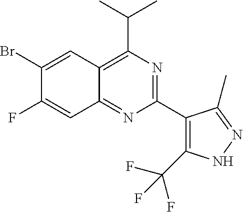 [SMILES: Cc1n[nH]c(C(F)(F)F)c1-c1nc(C(C)C)c2cc(Br)c(F)cc2n1]

Step A: N-(4-Bromo-3-fluorophenyl)-3-methyl-5-(trifluoromethyl)-pH-pyrazole-4-carboxamide. To a cooled solution (−78° C.) of methyl 3-methyl-5-(trifluoromethyl)-1H-pyrazole-4-carboxylate (780 mg, 3.7 mg) and 4-bromo-3-fluoroaniline (1780 mg, 9.4 mmol) in THF (15 mL) was added lithium bis(trimethylsilyl)amide solution (1.5M in THF, 10 mL, 15 mmol). The reaction mixture was allowed to warm to room temperature and stirred overnight. The reaction mixture was quenched with 1N HCl and stirred for 5 minutes. The reaction mixture was poured into water and extracted with ethyl acetate (3×). The combined organic layers were washed with brine, dried over Na₂SO₄, filtered, and concentrated under reduced pressure. The resulting residue was purified with flash column chromatography (SiO₂ eluting with a gradient of 0-50% ethyl acetate in heptane) to provide the title compound (1164 mg, 3.17 mmol, 84% yield) as an off-white solid. MS (ESI): mass calcd. for C₂₂H₂₁F₄N₅O, 364.98; m/z found, 366.0 [M+H]⁺. ¹H NMR (400 MHz, DMSO-d₆) δ=13.69 (s, 1H), 10.45 (s, 1H), 7.79 (dd, J=2.45, 11.25 Hz, 1H), 7.66 (t, J=8.56 Hz, 1H), 7.37 (dd, J=1.96, 8.80 Hz, 1H), 2.40 (s, 3H).

Step B: 6-Bromo-7-fluoro-4-isopropyl-2-(3-methyl-5-(trifluoromethyl)-1H-pyrazol-4-yl)quinazoline. Trifluoromethanesulfonic anhydride (1M in DCM) (0.45 mL, 1 M, 0.45 mmol) was added to N-(4-bromo-3-fluorophenyl)-3-methyl-5-(trifluoromethyl)-1H-pyrazole-4-carboxamide (150 mg, 0.41 mmol) and 2-chloropyridine (0.05 mL, 0.49 mmol) in dichloroethane (2.2 mL) at −78° C., over 1 minute. After 5 mins, the reaction mixture was warmed to ° C. Isobutyronitrile (0.04 mL, 0.45 mmol) was added to the reaction mixture. The reaction mixture was warmed to room temperature for 5 mins and then heated under microwave irradiation at 140° C. for 20 mins. The reaction mixture was cooled and 1N NaOH (1 mL) was slowly added. The reaction mixture was diluted with DCM, and the organic layer was washed with brine, dried over Na₂SO₄, and concentrated under reduced pressure. Purification by flash column chromatography (SiO2 12G 0-50% heptane/ethyl acetate) yielded the title compound (40 mg, 0.096 mmol, 23% yield) as a yellow oil, mixed with an inseparable impurity and used in the next step without further purification. MS (ESI): mass calcd. C₁₆H₁₃BrF₄N₄, 416.03; m/z found 417.0 [M+H]+.

### Intermediate 18: 2-Chloro-4-methyl-3-(4,4,5,5-tetramethyl-1,3,2-dioxaborolan-2-yl)pyridine

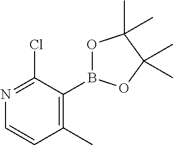 [SMILES: Cc1ccnc(Cl)c1B1OC(C)(C)C(C)(C)O1]

To a mixture of 3-bromo-2-chloro-4-methylpyridine (1 g, 4.84 mmol, 1.0 eq.) in dioxane (40 mL) was added B₂Pin₂ (1.23 g, 4.8 mmol, 1.0 eq.), Pd(dppf)Cl₂ (354.4 mg, 484.34 μmol, 0.1 eq.) and KOAc (1.43 g, 14.5 mmol, 3.0 eq.). The resulting reaction mixture was stirred at 100° C. for 12 hours under N₂. The reaction mixture was filtered and concentrated under vacuum. The resulting residue was purified by column chromatography (40 g, 0-10% ethyl acetate in petroleum ether) to give the title compound (630 mg, 2.44 mmol, 50% yield) as white solid. MS (ESI): mass calcd. for C₁₂H₁₇BclNO₂, 253.10; m/z found, 254.00 [M+H]⁺. ¹H NMR (400 MHz, CDCl₃) δ=8.22 (d, J=5.0 Hz, 1H), 7.00 (d, J=5.0 Hz, 1H), 2.38 (s, 3H), 1.42 (s, 12H) ppm.

### Intermediate 19: 6-Bromo-7-fluoro-4-(1,1,1-trifluoropropan-2-yl)quinolin-2-ol

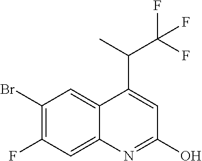 [SMILES: CC(c1cc(O)nc2cc(F)c(Br)cc12)C(F)(F)F]

Step A. 2,2-Dimethyl-5-(3,3,3-trifluoro-1-hydroxy-2-methylpropylidene)-1,3-dioxane-4,6-dione. To a solution of 3,3,3-trifluoro-2-methylpropanoic acid (4 g, 28.15 mmol, 1 eq.), 2,2-dimethyl-1,3-dioxane-4,6-dione (4.10 g, 28.43 mmol, 1.01 eq.) and DMAP (5.16 g, 42.23 mmol, 1.5 eq.) in DCM (80 mL) was added DCC (6.10 g, 29.56 mmol, 1.05 eq.) in DCM (20 mL) by dropwise at 0° C. The reaction mixture was stirred at 20° C. for 16 hours. The reaction mixture was diluted with DCM (200 mL), washed with 1M HCl (80 mL), then extracted with DCM (200 mL). The combined organic layer was dried over anhydrous Na₂SO₄ and concentrated under reduced pressure to give the title compound (7.3 g, 27.22 mmol, 97% yield) as yellow solid. ¹H NMR (400 MHz, CDCl₃) δ=15.65 (br s, 1H), 5.15-5.02 (m, 1H), 1.77 (s, 6H), 1.51 (d, J=7.2 Hz, 3H); ¹⁹F NMR (376 MHz, CDCl₃) δ=−68.34 (3F) ppm.

Step B. Methyl 5,5,5-trifluoro-4-methyl-3-oxopentanoate and methyl 5,5,5-trifluoro-3-hydroxy-4-methylpent-2-enoate. 2,2-Dimethyl-5-(3,3,3-trifluoro-1-hydroxy-2-methylpropylidene)-1,3-dioxane-4,6-dione (7.3 g, 27.22 mmol) was added to MeOH (50 mL). Then the reaction mixture was refluxed at 65° C. for 5 hours. The reaction mixture was concentrated under reduced pressure. The resulting residue was purified by flash column chromatography (SiO₂, gradient elution: 0-50% petroleum ether in ethyl acetate) to give the title compound (4.9 g, 24.73 mmol, 91% yield) as light brown oil. ¹H NMR (400 MHz, CDCl₃) δ=12.11 (s, 0.3H), 5.19 (s, 0.3H), 3.79-3.76 (m, 3H), 3.63 (d, J=8.0 Hz, 1H), 3.59-3.49 (m, 0.8H), 3.04-2.94 (m, 0.3H), 1.42-1.36 (m, 3H); ¹⁹F NMR (376 MHz, CDCl₃) δ=−68.26 (3F), −70.22 (3F) ppm.

Step C. N-(4-Bromo-3-fluorophenyl)-5,5,5-trifluoro-4-methyl-3-oxopentanamide and N-(4-bromo-3-fluorophenyl)-5,5,5-trifluoro-3-hydroxy-4-methylpent-2-enamide. A mixture of methyl 5,5,5-trifluoro-4-methyl-3-oxopentanoate and methyl 5,5,5-trifluoro-3-hydroxy-4-methylpent-2-enoate (4.9 g, 24.73 mmol, 1 eq.) was added to 4-bromo-3-fluoroaniline (4.7 g, 24.73 mmol, 1 eq.), then heated to 110° C. for 2 hours. The reaction mixture was purified by flash column chromatography (SiO₂, gradient elution: 0-30% petroleum ether in ethyl acetate) to give the title compound (1.6 g, 4.49 mmol, 18% yield) as light brown gum. ¹H NMR (400 MHz, CDCl₃) δ=13.51 (br s, 0.3H), 8.88 (br s, 0.5H), 7.66-7.56 (m, 1H), 7.52-7.45 (m, 1H), 7.13-6.99 (m, 1H), 5.14 (s, 0.3H), 3.75 (s, 1H), 3.55-3.43 (m, 0.6H), 2.99 (td, J=7.8, 15.7 Hz, 0.3H), 1.45-1.40 (m, 3H); ¹⁹F NMR (376 MHz, CDCl₃) δ=−68.15 (3F), −70.11 (3F), −104.81 (1F), −104.87 (1F) ppm.

Step D. 6-Bromo-7-fluoro-4-(1,1,1-trifluoropropan-2-yl)quinolin-2-ol. The mixture of N-(4-bromo-3-fluorophenyl)-5,5,5-trifluoro-4-methyl-3-oxopentanamide and N-(4-bromo-3-fluorophenyl)-5,5,5-trifluoro-3-hydroxy-4-methylpent-2-enamide (1.5 g, 4.2 mmol) was added to conc. H₂SO₄ (9 mL). The reaction mixture was stirred at 80° C. overnight. The reaction mixture was poured into ice water, then alkalized with 2M, aq. NaOH (20 mL), and extracted with ethyl acetate (150 mL×2). The organic layers were dried over anhydrous Na₂SO₄, concentrated under reduced pressure to give the crude product. The crude product was purified by flash column chromatography (SiO₂, gradient elution: 0-80% petroleum ether in ethyl acetate) to give the title compound as light brown solid. (135 mg, 332.5 μmol, 8% yield). MS (ESI): mass calcd. For C₁₂H₈BrF₄NO, 336.97; m/z found, 339.7 [M+H]⁺. ¹H NMR (400 MHz, CDCl₃) δ=8.26 (d, J=7.0 Hz, 1H), 7.23 (d, J=9.3 Hz, 1H), 6.73 (s, 1H), 4.48-4.35 (m, 1H), 1.59-1.55 (m, 3H); ¹⁹F NMR (376 MHz, CDCl₃) δ=−71.60 (3F), −104.58 (1F) ppm.

### Intermediate 20: 6-Bromo-7-fluoroquinoline-2,4-diol

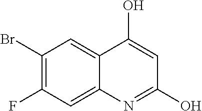 [SMILES: Oc1cc(O)c2cc(Br)c(F)cc2n1]

Step A. Methyl 2-amino-5-bromo-4-fluorobenzoate. To a stirred solution of 2-amino-5-bromo-4-fluorobenzoic acid (23 g, 98.28 mmol) and DMF (718.37 mg) in MeOH (250 mL) was added SOCl₂ (116.93 g, 982.81 mmol, 71.30 mL) drop-wise at 0° C. The reaction mixture was stirred at 60° C. for 72 hrs. The reaction mixture was concentrated under reduced pressure. The resulting residue was poured into sat.Na₂CO₃ (600 mL) and extracted with ethyl acetate (200 mL×2). The combined organic phase was washed with brine (300 mL×2), dried with anhydrous Na₂SO₄, filtered, and concentrated under reduced pressure. The resulting residue was purified by flash column chromatography (SiO₂, Petroleum ether/Ethyl acetate=100/1 to 8/1) to give the title compound (20 g, 77.21 mmol, 79% yield) as a white solid. MS (ESI): mass calcd For C₈H₇BrFNO₂, 247.0; m/z found, 250.5 [M+H]⁺. ¹H NMR (400 MHz, CDCl₃) δ=8.05 (d, J=7.9 Hz, 1H), 6.43 (d, J=10.3 Hz, 1H), 5.90 (s, 2H), 3.88 (s, 3H).

Step B. Methyl 2-(N-acetylacetamido)-5-bromo-4-fluorobenzoate. A solution of methyl 2-amino-5-bromo-4-fluorobenzoate (17.5 g, 70.6 mmol), DMAP (861.90 mg, 7.06 mmol, 0.1 eq) in Ac₂O (105 mL) and pyridine (105 mL) was stirred at 85° C. for 15 hrs. The reaction mixture was poured into 1 N HCl (500 mL) and extracted with DCM (500 mL×2). The combined organic phase was washed with brine (800 mL×2), dried with anhydrous Na₂SO₄, filtered, and concentrated under reduced pressure to afford the title compound which was used crude in the next step without further purification.

Step C. Methyl 2-acetamido-5-bromo-4-fluorobenzoate. The resulting residue from Step B was dissolved in MeOH (200 mL) and Na₂CO₃ (50 g) was added slowly. The resulting mixture was stirred at 20° C. for 1 hr. The reaction mixture was concentrated under reduced pressure. The resulting residue was purified by flash column chromatography (SiO₂, Petroleum ether/ethyl acetate=30/1 to 15/1) to give the title compound (16.5 g, 55.7 mmol, 79% yield) as a white solid. MS (ESI): mass calcd. for C₁₀H₉BrFNO₃, 289.0; m/z found, 290.4 [M+H]⁺. ¹H NMR (400 MHz, CDCl₃) δ=11.13 (s, 1H), 8.66 (d, J=11.5 Hz, 1H), 8.24 (d, J=7.6 Hz, 1H), 3.95 (s, 3H), 2.25 (s, 3H).

Step D. 6-Bromo-7-fluoroquinoline-2,4-diol. To a solution of methyl 2-acetamido-5-bromo-4-fluorobenzoate (3.6 g, 12.41 mmol) in THF (20 mL) was added KHMDS (1 M, 37.23 mL, 3 eq) drop-wise at −78° C., and the mixture was stirred at 20° C. for 3 hrs. The reaction mixture was poured into water (200 mL) and extracted with ethyl acetate (200 mL×2). The organic phase was concentrated under reduced pressure. The residue was purified by flash column chromatography (SiO₂, DCM/MeOH=10/1 to 2/1) to give the title compound (3.52 g, 12.00 mmol, 97% yield) as a light yellow solid. MS (ESI): mass calcd for C₉H₅BrFNO₂, 256.9; m/z found, 260.4 [M+H]⁺. ¹H NMR (400 MHz, DMSO-d₆) δ=11.35 (s, 1H), 7.97 (d, J=7.5 Hz, 1H), 7.16 (d, J=10.0 Hz, 1H), 5.73 (s, 1H), 3.16 (s, 2H).

### Example 1: 4-Ethyl-1-(7-fluoro-4-isopropyl-2-(2-methoxyphenyl)quinolin-6-yl)-3-(hydroxymethyl)-1H-1,2,4-triazol-5 (4H)-one

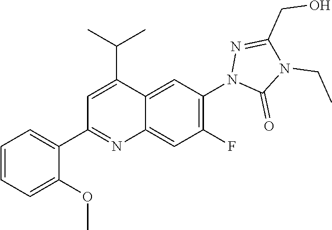 [SMILES: CCn1c(CO)nn(-c2cc3c(C(C)C)cc(-c4ccccc4OC)nc3cc2F)c1=O]

Step A. 3-((Benzyloxy)methyl)-4-ethyl-1-(7-fluoro-4-isopropyl-2-(2-methoxyphenyl)quinolin-6-yl)-1H-1,2,4-triazol-5 (4H)-one. (2-Methoxyphenyl)boronic acid (39 mg, 256.7 μmol), [1,1-bis(di-tert-butylphosphino)ferrocene]dichloropalladium(II) (Pd(dtbpf)Cl₂) (15 mg, 23.33 μmol) and K₂CO₃ (97 mg, 701.9 μmol) were added to the solution of 3-((benzyloxy)methyl)-1-(2-chloro-7-fluoro-4-isopropylquinolin-6-yl)-4-ethyl-1H-1,2,4-triazol-5 (4H)-one (Intermediate 2, 160 mg, 233.3 μmol) in a mixture of dioxane/H₂O (v/v, 5/1, 6 mL) at room temperature under N₂. The mixture was stirred at 85° C. for 16 hours under N₂. The mixture was diluted with H₂O (20 mL) and extracted with DCM (20 mL×3). The organic layer was dried over Na₂SO₄, filtered, and evaporated under reduced pressure. The crude product was purified by flash column chromatography (SiO₂, 0-80% ethyl acetate in petroleum ether) to give the title compound (110 mg, 197.4 μmol, 84.59% yield) as a white solid. MS (ESI): mass calcd. for C₃₁H₃₁FN₄O₃, 526.2; m/z found, 527.2 [M+H]⁺. ¹H NMR (400 MHz, CDCl₃) δ=8.23 (d, J=8.0 Hz, 1H), 7.88 (d, J=11.7 Hz, 1H), 7.81-7.73 (m, 2H), 7.40-7.24 (m, 6H), 7.07 (dt, J=1.0, 7.5 Hz, 1H), 6.98 (d, J=8.3 Hz, 1H), 4.57 (s, 2H), 4.49 (s, 2H), 3.87-3.82 (m, 2H), 3.81 (s, 3H), 3.69-3.57 (m, 1H), 1.38-1.31 (m, 9H); ¹⁹F NMR (376 MHz, CDCl₃) δ=−116.10 (s, 1F).

Step B. 4-Ethyl-1-(7-fluoro-4-isopropyl-2-(2-methoxyphenyl)quinolin-6-yl)-3-(hydroxymethyl)-1H-1,2,4-triazol-5 (4H)-one. BCl₃ (1 M solution in toluene, 0.79 mL, 0.79 mmol) was added to 3-((benzyloxy) methyl)-4-ethyl-1-(7-fluoro-4-isopropyl-2-(2-methoxyphenyl)quinolin-6-yl)-1H-1,2,4-triazol-5 (4H)-one (110 mg, 197.4 μmol) in DCM (5 mL) at −78° C. under N₂. The mixture was stirred at −78° C. for 1 hour. The mixture was quenched with MeOH (2 mL) at −78° C. and stirred for 0.5 hour. After 0.5 hours, the mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 50-80% B in A over 7 min, flow rate: 25 mL/min) to give the title compound (45 mg, 102.5 μmol, 51.95% yield) as an off-white powder. MS (ESI): mass calcd. for C₂₄H₂₅FN₄O₃, 436.2; m/z found, 437.2 [M+H]⁺. ¹H NMR (400 MHz, CDCl₃) δ=8.23 (d, J=8.1 Hz, 1H), 7.88 (d, J=11.8 Hz, 1H), 7.79-7.71 (m, 2H), 7.41-7.34 (m, 1H), 7.06 (dt, J=0.9, 7.5 Hz, 1H), 6.98 (d, J=8.2 Hz, 1H), 4.59 (br d, J=3.9 Hz, 2H), 3.85 (m, J=7.3 Hz, 2H), 3.80 (s, 3H), 3.62 (td, J=6.9, 13.6 Hz, 1H), 2.78 (br s, 1H), 1.38-1.32 (m, 9H); ¹⁹F NMR (376 MHz, CDCl₃) δ=−119.67 (s, 1F).

### Example 2: 1-(2-(3-Chloro-5-methyl-1H-pyrazol-4-yl)-7-fluoro-4-isopropylauinolin-6-yl)-4-ethyl-3-(hydroxymethyl)-1H-1,2,4-triazol-5 (4H)-one

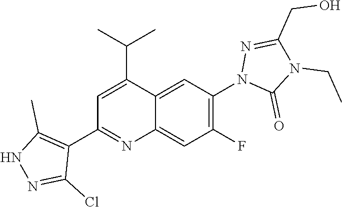 [SMILES: CCn1c(CO)nn(-c2cc3c(C(C)C)cc(-c4c(Cl)n[nH]c4C)nc3cc2F)c1=O]

Step A. 3-((Benzyloxy)methyl)-1-(2-(3-chloro-5-methyl-1H-pyrazol-4-yl-7-fluoro-4-isopropylquinolin-6-yl-4-ethyl-1H-1,2,4-triazol-5 (4H)-one. To a solution of 3-chloro-5-methyl-4-(4,4,5,5-tetramethyl-1,3,2-dioxaborolan-2-yl)-1H-pyrazole (Intermediate 3, 250 mg, 496 μmol), 3-((benzyloxy)methyl)-1-(2-chloro-7-fluoro-4-isopropylquinolin-6-yl)-4-ethyl-1H-1,2,4-triazol-5 (4H)-one (Intermediate 2, 235 mg, 413.33 μmol) and K₂CO₃ (114 mg, 826.65 μmol) in dioxane/H₂O (v/v, 5/1, 2 mL) was added Pd(dppf)Cl₂ (33.8 mg, 41.33 μmol) under N₂. The reaction mixture was stirred at 80° C. overnight and subsequently cooled to room temperature. The reaction mixture was diluted with ethyl acetate (20 mL), washed with brine (10 mL), and the aqueous layer was extracted with ethyl acetate (20 mL×3). The organic layers were dried over Na₂SO₄, filtered, and concentrated under reduced pressure. The crude product was purified by flash column chromatography (SiO₂, petroleum ether/ethyl acetate=I/O to 7/3) to give the title compound (150 mg, 272.76 μmol, 65.99% yield) as a yellow solid. MS (ESI): mass calcd. for C₂₁H₂₉ClFN₆O₂, 534.2; m/z found, 535.2 [M+H]⁺. ¹H NMR (400 MHz, CDCl₃) S=10.01 (s, 1H), 8.29 (d, J=7.7 Hz, 1H), 7.91-7.78 (m, 2H), 7.40 (d, J=2.6 Hz, 5H), 4.65 (s, 2H), 4.57 (s, 2H), 3.91 (d, J=7.3 Hz, 2H), 3.77-3.60 (m, 1H), 2.65 (d, J=3.5 Hz, 3H), 1.50-1.37 (m, 9H); ¹⁹F NMR (376 MHz, CDCl₃) δ=−119.29 (d, J=10.3 Hz, 1F).

Step B. 14243-Chloro-5-methyl-1H-pyrazol-4-yl)-7-fluoro-4-isopropylquinolin-6-yl)-4-ethyl-3-(hydroxymethyl)-1H-1,2,4-triazol-5 (4H)-one. BCl₃ (1 M in toluene, 1.36 mL, 1.36 mmol) was added to 3-((benzyloxy) methyl)-1-(2-(3-chloro-5-methyl-1H-pyrazol-4-yl)-7-fluoro-4-isopropylquinolin-6-yl)-4-ethyl-1H-1,2,4-triazol-5 (4H)-one (150 mg, 272.8 μmol) in DCM (6 mL) at −78° C. under N₂. The mixture was stirred at −78° C. for 1 hour. The mixture was quenched with MeOH (2 mL) at −78° C. and stirred for 0.5 hour. After 0.5 hour, the mixture was warmed to room temperature, diluted with DCM (12 mL) and washed with sat. aq. NaHCO₃ (15 mL). The organic layer was dried with Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 30-60% B in A over 7 min, flow rate: 25 mL/min) to give the title compound (70 mg, 157.3 μmol, 57.69% yield) as a white powder. MS (ESI): mass calcd. for C₂₁H₂₂ClFN₆O₂, 444.1; m/z found, 445.1 [M+H]⁺. ¹H NMR (400 MHz, CDCl₃) δ=13.24 (br s, 1H), 8.35 (d, J=8.1 Hz, 1H), 7.90 (d, J=11.6 Hz, 1H), 7.80 (s, 1H), 5.80 (t, as J=5.7 Hz, 1H), 4.50 (d, J=5.6 Hz, 2H), 3.81 (m, J=7.1 Hz, 2H), 3.72 (td, J=6.7, 13.6 Hz, 1H), 2.56 (s, 3H), 1.36 (d, J=6.8 Hz, 6H), 1.30 (t, J=7.1 Hz, 3H); ¹⁹F NMR (376 MHz, CDCl₃) δ=(s, 1F).

### Example 3: 4-Ethyl-1-(7-fluoro-4-isopropyl-2-(5-methyl-3-(trifluoromethyl)-1H-pyrazol-4-yl) quinolin-6-yl)-3-(hydroxymethyl)-1H-1,2,4-triazol-5 (4H)-one

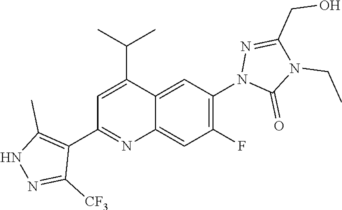 [SMILES: CCn1c(CO)nn(-c2cc3c(C(C)C)cc(-c4c(C(F)(F)F)n[nH]c4C)nc3cc2F)c1=O]

Step A. 3-((Benzyloxy)methyl)-4-ethyl-1-(7-fluoro-4-isopropyl-2-(5-methyl-3-(trifluoromethyl)-1H-pyrazol-4-yl)quinolin-6-yl)-1H-1,2,4-triazol-5 (4H)-one. To a solution of 5-methyl-4-(4,4,5,5-tetramethyl-1,3,2-dioxaborolan-2-yl)-3-(trifluoromethyl)-1H-pyrazole (Intermediate 4, 45 mg, 129.7 μmol), 3-((benzyloxy)methyl)-1-(2-chloro-7-fluoro-4-isopropylquinolin-6-yl)-4-ethyl-1H-1,2,4-triazol-5 (4H)-one (Intermediate 2, 74 mg, 129.7 μmol) and K₂CO₃ (54 mg, 389.0 μmol) in a mixture of dioxane/H₂O (v/v, 5/1, 0.5 mL) was added [1,1-bis(di-tert-butylphosphino)ferrocene]dichloropalladium(II) (Pd(dtbpf)Cl₂) (8.5 mg, 12.97 μmol) under N₂. The reaction mixture was stirred at 80° C. overnight and subsequently cooled to room temperature. The reaction mixture was diluted with ethyl acetate (15 mL), washed with brine (10 mL), and the aqueous layer was extracted with ethyl acetate (15 mL×3). The organic layers were dried over Na₂SO₄, filtered, and concentrated under reduced pressure. The solid was purified by flash column chromatography (SiO₂, petroleum ether/ethyl acetate=I/O to 7/3) to give the title compound (40 mg, 69.30 μmol, 53.44% yield) as a pale yellow solid. MS (ESI): mass calcd. for C₂₉H₂₈F₄N₆O₂, 568.2; m/z found, 569.3 [M+H]⁺. ¹H NMR (400 MHz, CDCl₃) δ=10.30 (s, 1H), 8.29 (d, J=7.9 Hz, 1H), 7.86 (d, J=11.5 Hz, 1H), 7.52 (s, 1H), 7.36 (s, 5H), 4.62 (s, 2H), 4.54 (s, 2H), 3.88 (m, J=7.2 Hz, 2H), 3.74-3.60 (m, 1H), 2.56 (s, 3H), 1.46-1.34 (m, 9H); ¹⁹F NMR (376 MHz, CDCl₃) δ=−59.85 (s, 1F), −118.59-118.77 (m, 1F).

Step B. 4-Ethyl-1-(7-fluoro-4-isopropyl-2-(5-methyl-3-(trifluoromethyl)-1H-pyrazol-4-yl)quinolin-6-yl)-3-(hydroxymethyl)-1H-1,2,4-triazol-5 (4H)-one. BCl₃ (1 M in toluene, 0.35 mL, 0.35 mmol) was added to 3-((benzyloxy)methyl)-4-ethyl-1-(7-fluoro-4-isopropyl-2-(5-methyl-3-(trifluoromethyl)-1H-pyrazol-4-yl)quinolin-6-yl)-1H-1,2,4-triazol-5 (4H)-one (40 mg, 69.30 μmol) in DCM (2 mL) at −78° C. under N₂. The mixture was stirred at −78° C. for 1 hour. The mixture was quenched with MeOH (0.5 mL) at −78° C. and stirred for 0.5 hour. After 0.5 hour, the mixture is warmed to room temperature, diluted with DCM (5 mL), and washed with sat. aq. NaHCO₃ (5 mL). The organic layer was dried over Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 40-70% B in A over 7 min, flow rate: 25 mL/min) to give the title compound (21 mg, 43.89 μmol, 63.34% yield) as a white powder. MS (ESI): mass calcd. for C₂₂H₂₂F₄N₆O₂, 478.2; m/z found, 479.2 [M+H]⁺. ¹H NMR (400 MHz, CDCl₃) δ=13.71 (br s, 1H), 8.40 (d, J=8.3 Hz, 1H), 7.91 (d, J=11.5 Hz, 1H), 7.54 (s, 1H), 5.81 (t, J=5.8 Hz, 1H), 4.52 (d, J=5.8 Hz, 2H), 3.83 (m, J=7.2 Hz, 2H), 3.75 (td, J=7.0, 13.7 Hz, 1H), 2.50 (s, 3H), 1.40-1.27 (m, 9H); ¹⁹F NMR (376 MHz, CDCl₃) δ=−58.27 (s, 1F), −118.53 (s, 1F).

### Example 4: 4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one

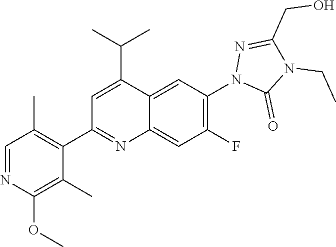 [SMILES: CCn1c(CO)nn(-c2cc3c(C(C)C)cc(-c4c(C)cnc(OC)c4C)nc3cc2F)c1=O]

Step A. 3-((Benzyloxy)methyl)-4-ethyl-1-(7-fluoro-4-isopropyl-2-(2-methoxy-3,5-dimethylpyridin-4-yl)quinolin-6-yl)-1H-1,2,4-triazol-5 (4H)-one. To a mixture of 3-((benzyloxy)methyl)-1-(2-chloro-7-fluoro-4-isopropylquinolin-6-yl)-4-ethyl-1H-1,2,4-triazol-5 (4H)-one (Intermediate 2, 150 mg, 303.9 μmol) in a mixture of dioxane/H₂O (v/v, 4/1, 5 mL) was added 2-methoxy-3,5-dimethyl-4-(4,4,5,5-tetramethyl-1,3,2-dioxaborolan-2-yl)pyridine (Intermediate 5, 120 mg, 454.8 μmol) and Na₂CO₃ (96.5 mg, 909.5 μmol). Pd(dppf)Cl₂ (22 mg, 30.60 μmol) was added under N₂. The resulting mixture was charged with N₂, the mixture was heated at 80° C. and stirred overnight. Then the mixture was cooled to room temperature. The mixture was diluted with water (3 mL) and extracted with ethyl acetate (5 mL×3). The combined organic layers were dried over Na₂SO₄, filtered, and concentrated under reduced pressure. The residue was purified by flash column chromatography (SiO₂, 0-37% ethyl acetate in petroleum ether) to give the title compound (160 mg, 287.95 μmol) as a colorless, sticky oil. MS (ESI): mass calcd. for C₃₂H₃₄FN₅O₃, 555.3; m/z found, 556.2 [M+H]⁺.

Step B. 4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one. 3-((Benzyloxy)methyl)-4-ethyl-1-(7-fluoro-4-isopropyl-2-(2-methoxy-3,5-dimethylpyridin-4-yl)quinolin-6-yl)-1H-1,2,4-triazol-5 (4H)-one (140 mg, 252 μmol) dissolved in DCM (4 mL) was cooled to −78° C., then BCl₃ (1.5 mL, 1.5 mmol) was added. The mixture was stirred at −78° C. for 3 hours. The mixture was quenched with MeOH (3 mL) and stirred at −78° C. for 0.5 h. After 0.5 hours, the mixture was warmed to room temperature, diluted with DCM (10 mL), and washed with sat. aq. NaHCO₃ (12 mL). The combined organic phase was washed with brine, dried over Na₂SO₄, filtered, and concentrated under reduced pressure. The residue was purified by preparative reversed phase HPLC (Stationary phase: Boston Prime C18, 5 μm, 150×30 mm; Mobile phase: water (0.04% NH₃H₂O+10 mM NH₄HCO₃) (A) —MeCN (B), gradient elution: 50-80% B in A over 7 min, flow rate: 25 mL/min) to give the title compound (85.6 mg, 183.88 μmol, 73.75% yield) as a white powder. MS (ESI): mass calcd. for C₂₅H₂₈FN₅O₃, 465.2; m/z found, 466.1 [M+H]⁺. ¹H NMR (400 MHz, CDCl₃) δ=8.36 (d, J=7.9 Hz, 1H), 7.97-7.83 (m, 2H), 7.20 (s, 1H), 4.68 (d, J=6.2 Hz, 2H), 3.97 (s, 3H), 3.93 (m, J=7.1 Hz, 2H), 3.72 (td, J=6.8, 13.6 Hz, 1H), 2.57 (t, J=6.1 Hz, 1H), 1.93 (d, J=8.8 Hz, 6H), 1.50-1.33 (m, 9H); ¹⁹F NMR (376 MHz, CDCl₃) δ=−118.13 (dd, J=8.4, 12.1 Hz, 1F).

### Example 5: 4-Ethyl-1-(7-fluoro-4-isopropyl-2-(pentan-3-yloxy)quinolin-6-yl)-3-(hydroxymethyl)-1H-1,2,4-triazol-5 (4H)-one

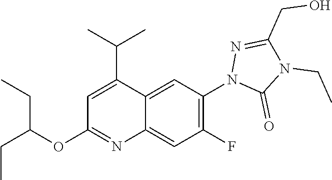 [SMILES: CCC(CC)Oc1cc(C(C)C)c2cc(-n3nc(CO)n(CC)c3=O)c(F)cc2n1]

Step A. 3-((Benzyloxy)methyl)-4-ethyl-1-(7-fluoro-4-isopropyl-2-(pentan-3-yloxy)quinolin-6-yl)-1H-1,2,4-triazol-5 (4H)-one. NaH (60% in mineral oil, 50 mg, 1.26 mmol) was slowly added into 3-pentanol (2 mL) at 0° C. After addition, the mixture was stirred at 0° C. for 0.5 hour. To the mixture was slowly added 3-((benzyloxy)methyl)-1-(2-chloro-7-fluoro-4-isopropylquinolin-6-yl)-4-ethyl-1H-1,2,4-triazol-5 (4H)-one (Intermediate 2, 120 mg, 252 μmol) at 0° C. The mixture was stirred at 50° C. for 16 hours. The mixture was diluted with H₂O (20 mL) and extracted with DCM (20 mL×3). The organic layer was dried over Na₂SO₄, filtered, and evaporated under reduced pressure to give the title compound (150 mg crude) as a yellow oil. MS (ESI): mass calcd. for C₂₉H₃₅FN₄O₃, 506.3; m/z found, 507.3 [M+H]⁺.

Step B. 4-Ethyl-1-(7-fluoro-4-isopropyl-2-(pentan-3-yloxy)quinolin-6-yl)-3-(hydroxymethyl)-1H-1,2,4-triazol-5 (4H)-one. BCl₃ (1 M solution in toluene, 1.2 mL, 1.2 mmol) was added to 3-((benzyloxy)methyl)-4-ethyl-1-(7-fluoro-4-isopropyl-2-(pentan-3-yloxy)quinolin-6-yl)-1H-1,2,4-triazol-5 (4H)-one (150 mg crude) in DCM (6 mL) at −78° C. under N₂. The mixture was stirred at −78° C. for 1 hour. The mixture was quenched with MeOH (3 mL) at −78° C., stirred for 0.5 hour. After 0.5 hours, the mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 70-100% B in A over 7 min, flow rate: 25 mL/min) to give the title compound (47 mg, 112.48 μmol, 44.65% yield of two steps) as a yellow sticky oil. MS (ESI): mass calcd. for C₂₂H₂₉FN₄O₃, 416.2; m/z found, 417.2 [M+H]⁺. ¹H NMR (400 MHz, CDCl₃) δ=8.01 (d, J=8.1 Hz, 1H), 7.51 (d, J=11.8 Hz, 1H), 6.69 (s, 1H), 5.24 (m, J=6.0 Hz, 1H), 4.61 (d, J=6.4 Hz, 2H), 3.85 (m, J=7.3 Hz, 2H), 3.45 (td, J=6.8, 13.6 Hz, 1H), 2.18 (t, J=6.4 Hz, 1H), 1.73-1.63 (m, 4H), 1.36 (t, J=7.3 Hz, 3H), 1.29 (d, J=6.8 Hz, 6H), 0.90 (t, J=7.5 Hz, 6H); ¹⁹F NMR (376 MHz, CDCl₃) δ=−120.62 (s, 1F).

### Example 6: 1-(2-Cyclobutoxy-7-fluoro-4-isopropylauinolin-6-yl)-4-ethyl-3-(hydroxymethyl)-1H-1,2,4-triazol-5 (4H)-one

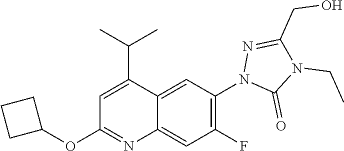 [SMILES: CCn1c(CO)nn(-c2cc3c(C(C)C)cc(OC4CCC4)nc3cc2F)c1=O]

Step A. 3-((Benzyloxy)methyl)-1-(2-cyclobutoxy-7-fluoro-4-isopropylquinolin-6-yl)-4-ethyl-1H-1,2,4-triazol-5 (4H)-one. NaH (60% in mineral oil, 50 mg, 1.26 mmol) was added slowly into cyclobutanol (2 mL) at 0° C., and stirred at 0° C. for 0.5 hour. To the reaction mixture was slowly added 3-((benzyloxy)methyl)-1-(2-chloro-7-fluoro-4-isopropylquinolin-6-yl)-4-ethyl-1H-1,2,4-triazol-5 (4H)-one (Intermediate 2, 120 mg, 2512 μmol) at 0° C. The resulting reaction mixture was stirred at 50° C. for 16 hours. The mixture was diluted with H₂O (20 mL) and extracted with DCM (20 mL×3). The organic layer was dried over Na₂SO₄, filtered, and evaporated under reduced pressure to give the title compound (200 mg crude) as a yellow oil. MS (ESI): mass calcd. for C₂₈H₃₁FN₄O₃, 490.2; m/z found, 491.2 [M+H]⁺.

Step B. 1-(2-Cyclobutoxy-7-fluoro-4-isopropylquinolin-6-yl)-4-ethyl-3-(hydroxymethyl)-1H-1,2,4-triazol-5 (4H)-one. BCl₃ (1 M solution in toluene, 1.8 mL, 1.8 mmol) was added to 3-((benzyloxy)methyl)-1-(2-cyclobutoxy-7-fluoro-4-isopropylquinolin-6-yl)-4-ethyl-1H-1,2,4-triazol-5 (4H)-one (200 mg crude) in DCM (5 mL) at −78° C. under N₂. The mixture was stirred at −78° C. for 1 hour. The mixture was quenched with MeOH (4 mL) at −78° C., stirred at same temperature for 0.5 hour. After 0.5 hours, the mixture was diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 60-90% B in A over 7 min, flow rate: 25 mL/min) to give the title compound (85 mg, 212.3 μmol, 84.25% yield of two steps) as a yellow sticky oil. MS (ESI): mass calcd. for C₂₁H₂₅FN₄O₃, 400.2; m/z found, 401.1 [M+H]⁺. ¹H NMR (400 MHz, CDCl₃) δ=8.12 (d, J=8.1 Hz, 1H), 7.61 (d, J=11.8 Hz, 1H), 6.79 (s, 1H), 5.41 (m, J=7.4 Hz, 1H), 4.70 (d, J=6.2 Hz, 2H), 3.94 (m, J=7.2 Hz, 2H), 3.55 (td, J=6.9, 13.6 Hz, 1H), 2.60-2.49 (m, 2H), 2.30-2.14 (m, 3H), 1.94-1.68 (m, 2H), 1.45 (t, J=7.2 Hz, 3H), 1.37 (d, J=6.8 Hz, 6H); ¹⁹F NMR (376 MHz, CDCl₃) δ=−120.27 (s, 1F).

### Example 7: 1-(2-(3-Chloro-2-methoxy-5-methylpyridin-4-yl)-7-fluoro-4-isopropylquinolin-6-yl)-4-ethyl-3-(hydroxymethyl)-1H-1,2,4-triazol-5 (4H)-one

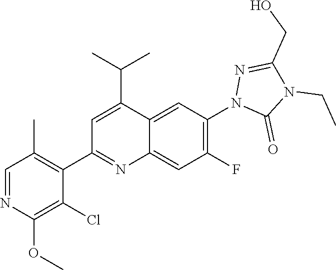 [SMILES: CCn1c(CO)nn(-c2cc3c(C(C)C)cc(-c4c(C)cnc(OC)c4Cl)nc3cc2F)c1=O]

Step A. 3-((Benzyloxy)methyl)-1-(2-(3-chloro-2-methoxy-5-methylpyridin-4-yl)-7-fluoro-4-isopropylquinolin-6-yl)-4-ethyl-1H-1,2,4-triazol-5 (4H)-one and 3-((benzyloxy)methyl)-4-ethyl-1-(7-fluoro-4-isopropyl-2-(2-methoxy-5-methylpyridin-4-yl)quinolin-6-yl)-1H-1,2,4-triazol-5 (4H)-one. To a solution of 3-((benzyloxy)methyl)-1-(2-chloro-7-fluoro-4-isopropylquinolin-6-yl)-4-ethyl-1H-1,2,4-triazol-5 (4H)-one (Intermediate 2, 179 mg, 393.04 μmol), 3-chloro-2-methoxy-5-methyl-4-(4,4,5,5-tetramethyl-1,3,2-dioxaborolan-2-yl)pyridine (Intermediate 6, 217 mg, 765.8 μmol) and Na₂CO₃ (125 mg, 1.18 mmol) in a mixture of dioxane/H₂O (v/v, 5/1, 3.3 mL) was added Pd(dppf)Cl₂.DCM (32 mg, 39.33 μmol) under N₂. The reaction mixture was stirred at 80° C. overnight. The mixture was diluted H₂O (20 mL) and extracted with ethyl acetate (25 ml×3). The combined organic phase was dried over anhydrous Na₂SO₄, filtered, and concentrated under reduced pressure. The crude product was purified by flash column chromatography (SiO₂, gradient elution: 0-30% ethyl acetate in petroleum ether) to give a white solid. It was further purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 75-100% B in A over 7 min, flow rate: 25 mL/min) to give two compounds The first eluting compound (75 mg, 125.8 μmol, 31.99% yield) was isolated as a white solid. MS (ESI): mass calcd. for C₃₁H₃₁ClFN₅O₃, 575.2; m/z found, 576.1 [M+H]⁺. ¹H NMR (400 MHz, CDCl₃) δ=8.33 (d, J=7.9 Hz, 1H), 7.97 (s, 1H), 7.88 (d, J=11.2 Hz, 1H), 7.36-7.29 (m, 5H), 7.22-7.20 (m, 1H), 4.59 (s, 2H), 4.51 (s, 2H), 4.01 (s, 3H), 3.85 (m, J=7.1 Hz, 2H), 3.70 (m, J=6.8 Hz, 1H), 1.99 (s, 3H), 1.40-1.33 (m, 9H); ¹⁹F NMR (376 MHz, CDCl₃) δ=−117.47-118.11 (m, 1F).

The second eluting compound, identified as a side product, (20 mg, 35.87 μmol, 9.13% yield) was isolated as a white powder. MS (ESI): mass calcd. for C₃₁H₃₂FN₅O₃, 541.2; m/z found, 542.1 [M+H]⁺.

Step B. 1-(2-(3-Chloro-2-methoxy-5-methylpyridin-4-yl)-7-fluoro-4-isopropylauinolin-6-yl)-4-ethyl-3-(hydroxymethyl)-1H-1,2,4-triazol-5 (4H)-one. BCl₃ (1 M solution in toluene, 0.63 mL, 0.63 mmol) was added to a stirred solution of 3-((benzyloxy)methyl)-1-(2-(3-chloro-2-methoxy-5-methylpyridin-4-yl)-7-fluoro-4-isopropylquinolin-6-yl)-4-ethyl-1H-1,2,4-triazol-5 (4H)-one (75 mg, 125.8 μmol) in DCM (4 mL) at −78° C., and the mixture was stirred at −78° C. for 1 hour. The mixture was quenched with MeOH (1 mL) at −78° C. and stirred at −78° C. for 0.5 hour. After 0.5 hours, the mixture was warmed to room temperature, diluted with DCM (10 mL) and washed with sat. aq. NaHCO₃ (12 mL). The organic phase was dried with anhydrous Na₂SO₄, filtered, and concentrated under reduced pressure. The crude product was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 50-80% B in A over 7 min, flow rate: 25 mL/min) to give the title compound (42 mg, 86.43 μmol, 68.73% yield) as a white powder. MS (ESI): mass calcd. for C₂₄H₂₅ClFN₅O₃, 485.2; m/z found, 486.1 [M+H]⁺. ¹H NMR (400 MHz, CDCl₃) δ=8.42 (d, J=7.8 Hz, 1H), 8.04 (s, 1H), 7.95 (d, J=11.3 Hz, 1H), 7.32 (s, 1H), 4.73 (d, J=6.0 Hz, 2H), 4.08 (s, 3H), 3.97 (m, J=7.3 Hz, 2H), 3.83-3.71 (m, 1H), 2.38 (t, J=6.3 Hz, 1H), 2.06 (s, 3H), 1.51-1.40 (m, 9H); ¹⁹F NMR (376 MHz, CDCl₃) δ=−117.92 (s, 1F).

### Example 8: 4-Ethyl-1-(7-fluoro-4-isopropyl-2-(2-methoxy-5-methylpyridin-4-yl)quinolin-6-yl)-3-(hydroxymethyl)-1H-1,2,4-triazol-5 (4H)-one

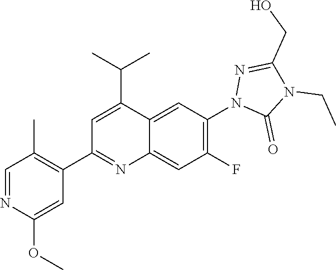 [SMILES: CCn1c(CO)nn(-c2cc3c(C(C)C)cc(-c4cc(OC)ncc4C)nc3cc2F)c1=O]

BCl₃ (1 M solution in toluene, 0.18 mL, 0.18 mmol) was added to a stirred solution of 3-((benzyloxy)methyl)-4-ethyl-1-(7-fluoro-4-isopropyl-2-(2-methoxy-5-methylpyridin-4-yl)quinolin-6-yl)-1H-1,2,4-triazol-5 (4H)-one (side product, example 7, step A, 20 mg, 35.87 μmol) in DCM (3 mL) at −78° C., and the mixture was stirred at −78° C. for 1 hour. The mixture was quenched with MeOH (1 mL) at −78° C. and stirred at −78° C. for 0.5 hour. After 0.5 hours, the mixture was warmed to room temperature, diluted with DCM (10 mL) and washed with sat. aq. NaHCO₃ (12 mL). The organic phase was dried with anhydrous Na₂SO₄, filtered, and concentrated under reduced pressure. The crude product was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 45-75% B in A over 7 min, flow rate: 25 mL/min) to give the title compound (8 mg, 17.72 μmol, 49.40% yield) as a white powder. MS (ESI): mass calcd. for C₂₄H₂₆FN₅O₃, 451.2; m/z found, 452.1 [M+H]⁺. ¹H NMR (400 MHz, CDCl₃) δ=8.39 (d, J=8.0 Hz, 1H), 8.14 (s, 1H), 7.96 (d, J=11.3 Hz, 1H), 7.45 (s, 1H), 6.93 (s, 1H), 4.73 (s, 2H), 4.00 (s, 3H), 3.99-3.94 (m, 2H), 3.83-3.69 (m, 1H), 2.33 (s, 4H), 1.50-1.42 (m, 9H); ¹⁹F NMR (376 MHz, CDCl₃) δ=−118.10 (s, 1F).

### Example 9: 4-Ethyl-1-(7-fluoro-4-isopropyl-2-(o-tolyl)quinolin-6-yl)-3-(hydroxymethyl)-1H-1,2,4-triazol-5 (4H)-one

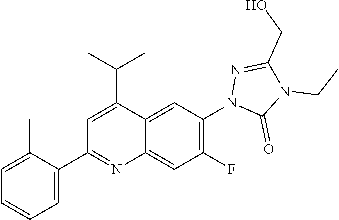 [SMILES: CCn1c(CO)nn(-c2cc3c(C(C)C)cc(-c4ccccc4C)nc3cc2F)c1=O]

Step A. 3-((Benzyloxy)methyl)-4-ethyl-1-(7-fluoro-4-isopropyl-2-(o-tolyl)quinolin-6-yl)-1H-1,2,4-triazol-5 (4H)-one. 2-Methylphenylboronic acid (428 mg, 3.15 mmol), K₂CO₃ (871 mg, 6.30 mmol), and [1,1-bis(di-tert-butylphosphino)ferrocene]dichloropalladium(II) (Pd(dtbpf)Cl₂) (137 mg, 210 μmol) were added into the solution of 3-((benzyloxy)methyl)-1-(2-chloro-7-fluoro-4-isopropylquinolin-6-yl)-4-ethyl-1H-1,2,4-triazol-5 (4H)-one (intermediate 2, 1 g, 2.20 mmol) in a mixture of dioxane/H₂O (v/v, 5/1, 15 mL) at room temperature under N₂. The mixture was stirred at 50° C. for 2 hours under N₂. The mixture was diluted with H₂O (50 mL) and extracted with ethyl acetate (60 mL×3). The combined organic phase was dried with anhydrous Na₂SO₄, filtered, and concentrated under reduced pressure. The crude product was purified by flash column chromatography (SiO₂, gradient elution: 0-50% ethyl acetate in petroleum ether) to give the title compound (930 mg, 1.82 mmol, 82.86% yield) as a white solid. MS (ESI): mass calcd. for C₃₁H₃₁FN₄O₂, 510.2; m/z found, 511.2 [M+H]⁺. ¹H NMR (400 MHz, CDCl₃) δ=8.31 (d, J=7.9 Hz, 1H), 7.92 (d, J=11.5 Hz, 1H), 7.52-7.47 (m, 1H), 7.43 (s, 1H), 7.39-7.29 (m, 8H), 4.62 (s, 2H), 4.54 (s, 2H), 3.89 (m, J=7.3 Hz, 2H), 3.78-3.66 (m, 1H), 2.39 (s, 3H), 1.42-1.36 (m, 9H); ¹⁹F NMR (376 MHz, CDCl₃) δ=−114.77-125.23 (m, 1F).

Step B. 4-Ethyl-1-(7-fluoro-4-isopropyl-2-(o-tolyl)quinolin-6-yl)-3-(hydroxymethyl)-1H-1,2,4-triazol-5 (4H)-one. BCl₃ (1 M solution in toluene, 1.18 mL, 1.18 mmol) was added to a stirred solution of 3-((benzyloxy)methyl)-4-ethyl-1-(7-fluoro-4-isopropyl-2-(o-tolyl)quinolin-6-yl)-1H-1,2,4-triazol-5 (4H)-one (120 mg, 235.0 μmol) in DCM (5 mL) at −78° C., and the mixture was stirred at −78° C. for 1 hour. The mixture was quenched with MeOH (1 mL) at −78° C. and stirred at −78° C. for 0.5 hour. After 0.5 hours, the mixture was warmed to room temperature, diluted with DCM (30 mL) and washed with sat. aq. NaHCO₃ (35 mL). The combined organic phase was dried with anhydrous Na₂SO₄, filtered, and concentrated under reduced pressure. The residue was purified by flash column chromatography (SiO₂, petroleum ether/ethyl acetate=1/1). It was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 55-85% B in A over 7 min, flow rate: 25 mL/min) to give the title compound (60 mg, 140.6 μmol, 59.84% yield) as a white powder. MS (ESI): mass calcd. for C₂₄H₂₅FN₄O₂, 420.2; m/z found, 421.2 [M+H]⁺. ¹H NMR (400 MHz, CDCl₃) δ=8.35 (d, J=8.0 Hz, 1H), 7.95 (d, J=11.6 Hz, 1H), 7.54-7.50 (m, 1H), 7.46 (s, 1H), 7.41-7.31 (m, 3H), 4.70 (d, J=6.2 Hz, 2H), 3.95 (m, J=7.2 Hz, 2H), 3.79-3.69 (m, 1H), 2.45-2.38 (m, 4H), 1.48-1.41 (m, 9H); ¹⁹F NMR (376 MHz, CDCl₃) δ=−118.97 (s, 1F).

### Example 10: 4-Ethyl-2-(7-fluoro-4-isopropyl-2-(o-tolyl)quinazolin-6-yl)-5-(hydroxymethyl)-2,4-dihydro-3H-1,2,4-triazol-3-one

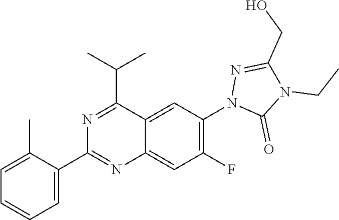 [SMILES: CCn1c(CO)nn(-c2cc3c(C(C)C)nc(-c4ccccc4C)nc3cc2F)c1=O]

Step A. 5-((Benzyloxy)methyl)-4-ethyl-2-(7-fluoro-4-isopropyl-2-(o-tolyl)quinazolin-6-yl)-2,4-dihydro-3H-1,2,4-triazol-3-one. The mixture of 6-chloro-7-fluoro-4-isopropyl-2-(o-tolyl)quinazoline (Intermediate 16, 214 mg, 0.68 mmol), (5-((benzyloxy)methyl)-4-ethyl-2,4-dihydro-3H-1,2,4-triazol-3-one) (Intermediate 1, 190.3 mg, 0.82 mmol), copper(I) iodide, trans-N,N′-dimethylcyclohexane-1,2-diamine (64 μL, 0.41 mmol), and potassium phosphate tribasic (259.7 mg, 1.22 mmol) in 1,4-dioxane (5 mL) was heated at 110° C. overnight. The reaction was partitioned between ethyl acetate and H₂O. The organic layer was dried over Na₂SO₄, filtered, and concentrated under reduced pressure. Purification by flash column chromatography (SiO₂ 4G 0-40% heptane/EA) yielded the title compound (52 mg, yield 14.951%) as a yellow solid. MS (ESI): mass calcd. C₃₀H₃₀FN₅O₂, 511.24; m/z found 512.3 [M+H]⁺. ¹H NMR (400 MHz, CDCl₃) δ=ppm 1.39-1.43 (m, 3H) 1.46-1.48 (m, 3H) 1.48-1.50 (m, 3H) 2.67 (s, 3H) 3.87-3.95 (m, 3H) 4.54-4.57 (m, 2H) 4.63-4.65 (m, 2H) 7.34-7.40 (m, 8H) 7.85 (d, J=11.25 Hz, 1H) 8.04 (br d, J=7.34 Hz, 1H) 8.42 (d, J=7.83 Hz, 1H).

Step B. 4-Ethyl-2-(7-fluoro-4-isopropyl-2-(o-tolyl)quinazolin-6-yl)-5-hydroxymethyl)-2,4-dihydro-3H-1,2,4-triazol-3-one. To a solution of 5-((benzyloxy)methyl)-4-ethyl-2-(7-fluoro-4-isopropyl-2-(o-tolyl)quinazolin-6-yl)-2,4-dihydro-3H-1,2,4-triazol-3-one (65 mg, 0.127 mmol) in DCM at −78° C. was added boron trichloride (0.634 mL, 1 M, 0.634 mmol). The mixture was stirred for 1 hour then carefully quenched by the dropwise addition of MeOH followed by water. The organics were extracted with DCM, washed with brine, dried over Na₂SO₄, filtered, and concentrated under reduced pressure. Purification by flash column chromatography (SiO₂, 12 g gold column, 20-40% EtOAc/heptane) yielded the title compound (11.5 mg, 0.027 mmol, 21% yield) as a white solid. MS (ESI): mass calcd. C₂₃H₂₄FN₅O₂, 421.19; m/z found 422.2 [M+H]⁺. ¹H NMR (400 MHz, CDCl₃) Shift 8.43 (d, J=7.83 Hz, 1H), 8.04 (d, J=7.34 Hz, 1H), 7.85 (d, J=10.76 Hz, 1H), 7.28-7.43 (m, 3H), 4.71 (d, J=6.36 Hz, 2H), 3.85-4.02 (m, 3H), 2.66 (s, 3H), 2.23 (s, 1H), 1.43-1.51 (m, 9H).

### Example 11: 4-Ethyl-2-(7-fluoro-4-isopropyl-2-(2-methoxy-4-methylpyridin-3-yl)quinolin-6-yl)-5-(hydroxymethyl)-2,4-dihydro-3H-1,2,4-triazol-3-one

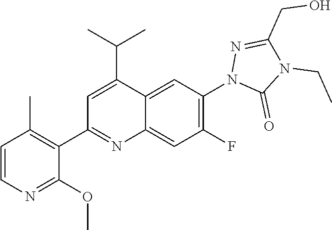 [SMILES: CCn1c(CO)nn(-c2cc3c(C(C)C)cc(-c4c(C)ccnc4OC)nc3cc2F)c1=O]

The title compound was prepared in a manner analogous to Example 1, Steps A-B, except using 2-methoxy-4-methyl-3-(4,4,5,5-tetramethyl-1,3,2-dioxaborolan-2-yl)pyridine (Intermediate 10) instead of (2-methoxyphenyl)boronic acid in Step A. MS (ESI): mass calcd. for C₂₄H₂₆FN₅O₃, 451.2; m/z found, 452.2 [M+H]⁺. ¹H NMR (400 MHz, CDCl₃) δ=8.28 (d, J=8.0 Hz, 1H), 8.04 (d, J=5.2 Hz, 1H), 7.85 (d, J=11.6 Hz, 1H), 7.28 (s, 1H), 6.81 (d, J=5.4 Hz, 1H), 4.63 (s, 2H), 3.87 (q, J=7.2 Hz, 2H), 3.81 (s, 3H), 3.69-3.60 (m, 1H), 2.40 (br s, 1H), 2.10 (s, 3H), 1.41-1.33 (m, 9H); ¹⁹F NMR (376 MHz, CDCl₃) δ=−119.08 (s, 1F).

### Example 12: 1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one

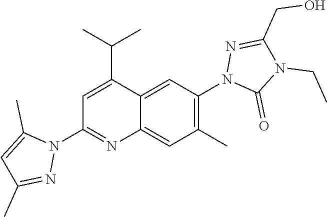 [SMILES: CCn1c(CO)nn(-c2cc3c(C(C)C)cc(-n4nc(C)cc4C)nc3cc2C)c1=O]

Step A. 3-(Benzyloxy)methyl)-1-(2-(3,5-dimethyl-1H-pyrazol-1-yl)-7-fluoro-4-isopropylquinolin-6-yl)-4-ethyl-1H-1,2,4-triazol-5 (4H)-one. To a cooled solution (0° C.) of 3,5-dimethyl-1H-pyrazole (20 mg, 208.05 μmol) in DMF (0.5 mL), was added NaH (60% purity, 10 mg, 250.02 μmol) under N₂. The reaction mixture was stirred at 0° C. for 30 minutes. 3-((Benzyloxy)methyl)-1-(2-chloro-7-fluoro-4-isopropylquinolin-6-yl)-4-ethyl-1H-1,2,4-triazol-5 (4H)-one (Intermediate 2, 118 mg, 208.05 μmol) in DMF (0.5 mL) was slowly added to the reaction mixture. The reaction mixture was stirred at 50° C. for 16 hours under N₂. The reaction mixture was diluted with ethyl acetate and washed with brine. The organic layer was separated, dried with Na₂SO₄, filtered, and concentrated under reduced pressure. The crude product was purified by flash column chromatography (SiO₂, petroleum ether/ethyl acetate=1/0 to 2/1), following by prep-TLC (SiO₂, DCM) to give the title compound (60 mg, 97.03 μmol, 46.64% yield) as white solid. MS (ESI): mass calcd. for C29H31FN602, 514.2; m/z found, 515.2 [M+H]⁺. ¹H NMR (400 MHz, CDCl₃) δ=8.26 (d, J=8.0 Hz, 1H), 8.06 (s, 1H), 7.76 (d, J=11.5 Hz, 1H), 7.44-7.30 (m, 5H), 6.06 (s, 1H), 4.64 (s, 2H), 4.56 (s, 2H), 3.91 (q, J=7.2 Hz, 2H), 3.72-3.61 (m, 1H), 2.80 (s, 3H), 2.35 (s, 3H), 1.47 (s, 3H), 1.46 (s, 3H), 1.40 (t, J=7.3 Hz, 3H); ¹⁹F NMR (376 MHz, CDCl₃) δ=−118.85 (s, 1F).

Step B. 1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one. BCl₃ (1 M solution in toluene, 0.58 mL, 0.58 mmol) was added to 3-((benzyloxy)methyl)-1-(2-(3,5-dimethyl-1H-pyrazol-1-yl)-7-fluoro-4-isopropylquinolin-6-yl)-4-ethyl-1H-1,2,4-triazol-5 (4H)-one (60 mg, 97.03 μmol) in DCM (1.5 mL) at −78° C. under N₂. The reaction mixture was quenched with MeOH (1 mL) at −78° C. and stirred at −78° C. for 0.5 hour. After 0.5 hours, the reaction mixture was warmed to room temperature, diluted with DCM (30 mL) and washed with sat. aq. NaHCO₃ (35 mL). The combined organic phase was dried with anhydrous Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 55-85% B in A over 7 min, flow rate: 25 mL/min) to give the title compound (21 mg, 49.47 μmol, 50.99% yield, 100% purity) as white powder. MS (ESI): mass calcd. for C₂₂H₂₅FN₆O₂, 424.2; m/z found, 425.1 [M+H]⁺. ¹H NMR (400 MHz, CDCl₃) S=8.24 (d, J=8.1 Hz, 1H), 8.05 (s, 1H), 7.75 (d, J=11.6 Hz, 1H), 6.05 (s, 1H), 4.70 (d, J=6.2 Hz, 2H), 3.94 (q, J=7.3 Hz, 2H), 3.66 (td, J=6.8, 13.7 Hz, 1H), 2.79 (s, 3H), 2.34 (s, 3H), 2.21 (br t, J=6.5 Hz, 1H), 1.49-1.38 (m, 9H); ¹⁹F NMR (376 MHz, CDCl₃) δ=−119.00 (s, 1F).

### Example 13: 1-(2-(Diethylamino)-7-fluoro-4-isopropylquinolin-6-yl)-4-ethyl-3-(hydroxymethyl)-1H-1,2,4-triazol-5 (4H)-one

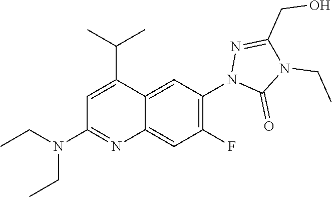 [SMILES: CCN(CC)c1cc(C(C)C)c2cc(-n3nc(CO)n(CC)c3=O)c(F)cc2n1]

Step A. 3-((Benzyloxy)methyl)-1-(2-(diethylamino)-7-fluoro-4-isopropylquinolin-6-yl)-4-ethyl-1H-1,2,4-triazol-5 (4H)-one. A solution consisting of 3-((benzyloxy)methyl)-1-(2-chloro-7-fluoro-4-isopropylquinolin-6-yl)-4-ethyl-1H-1,2,4-triazol-5 (4H)-one (Intermediate 2, 300 mg, 629.89 μmol), diethylamine (138 mg, 1.89 mmol) and conc. HCl (1 mL) in THF (5 mL) was stirred at 80° C. for 16 hours. The reaction mixture was cooled to room temperature, diluted with H₂O (10 mL) and extracted with DCM (10 mL×3). The combined organic layer was washed with brine, dried over Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 80-100% B in A over 7 min, flow rate: 25 mL/min) to give the title compound (90 mg, 170.66 μmol, 27.09% yield) as yellow solid. MS (ESI): mass calcd. for C₂₅H₃₄FN₅O₂, 491.3; m/z found, 492.3 [M+H]⁺. ¹H NMR (400 MHz, CDCl₃) δ=7.96 (d, J=8.3 Hz, 1H), 7.46-7.33 (m, 6H), 6.70 (s, 1H), 4.64 (s, 2H), 4.56 (s, 2H), 3.90 (q, J=7.2 Hz, 2H), 3.68 (q, J=7.1 Hz, 4H), 3.53 (td, J=6.8, 13.7 Hz, 1H), 1.43-1.35 (m, 9H), 1.27 (t, J=7.0 Hz, 6H); ¹⁹F NMR (376 MHz, CDCl₃) δ=−121.76-121.81 (m, 1F).

Step B. 1-(2-(Diethylamino)-7-fluoro-4-isopropylquinolin-6-yl)-4-ethyl-3-(hydroxymethyl)-1H-1,2,4-triazol-5 (4H)-one. BCl₃ (1 M solution in toluene, 0.9 mL, 0.9 mmol) was added to 3-((benzyloxy)methyl)-1-(2-(diethylamino)-7-fluoro-4-isopropylquinolin-6-yl)-4-ethyl-1H-1,2,4-triazol-5 (4H)-one (90 mg, 170.66 μmol) in DCM (5 mL) at −78° C. under N₂. The reaction mixture was stirred at −78° C. for 1 hour. The reaction mixture was quenched with MeOH (2 mL) at −78° C. and stirred for 0.5 hour. After 0.5 hours, the reaction mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Xtimate C18, 5 μm, 150×40 mm; Mobile phase: water (0.05% HCl) (A) —MeCN (B), gradient elution: 15-45% B in A over 7 min, flow rate: 30 mL/min) to give the crude compound (70 mg). Further purification by preparative reversed phase HPLC (Stationary phase: Phenomenex Gemini NX-C18, 4 μm, 150×40 mm; Mobile phase: water (0.05% NH₃H₂O+10 mM NH₄HCO₃) (A) —MeCN (B), gradient elution: 45-75% B in A over 7 min, flow rate: 30 mL/min) afforded the title compound (37 mg, 92.16 μmol, 54% yield) as off-white powder. MS (ESI): mass calcd. for C₂₁H₂₈FN₅O₂, 401.2; m/z found, 402.2 [M+H]⁺. ¹H NMR (400 MHz, CDCl₃) δ=7.95 (d, J=8.3 Hz, 1H), 7.43 (d, J=12.4 Hz, 1H), 6.70 (s, 1H), 4.67 (s, 2H), 3.92 (q, J=7.3 Hz, 2H), 3.67 (q, J=7.0 Hz, 4H), 3.52 (td, J=6.8, 13.7 Hz, 1H), 2.47 (br s, 1H), 1.43 (t, J=7.2 Hz, 3H), 1.36 (d, J=6.8 Hz, 6H), 1.27 (t, J=7.0 Hz, 6H); ¹⁹F NMR (376 MHz, CDCl₃) δ=−121.91 (s, 1F).

### Example 14: 4-Ethyl-1-(7-fluoro-4-isopropyl-2-(piperidin-1-yl)quinolin-6-yl)-3-(hydroxymethyl)-1H-1,2,4-triazol-5 (4H)-one

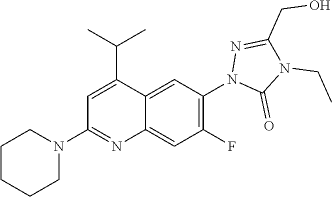 [SMILES: CCn1c(CO)nn(-c2cc3c(C(C)C)cc(N4CCCCC4)nc3cc2F)c1=O]

Step A. 3-((Benzyloxy)methyl)-4-ethyl-1-(7-fluoro-4-isopropyl-2-(piperidin-1-yl)quinolin-6-yl)-1H-1,2,4-triazol-5 (4H)-one. A solution consisting of 3-((benzyloxy)methyl)-1-(2-chloro-7-fluoro-4-isopropylquinolin-6-yl)-4-ethyl-1H-1,2,4-triazol-5 (4H)-one (Intermediate 2, 100 mg, 209.96 μmol) in piperidine (1 mL) at room temperature was stirred at 50° C. for 16 hours. The reaction mixture was diluted with H₂O (20 mL) and extracted with DCM (30 mL×3). The organic layer was dried over Na₂SO₄, filtered, and evaporated under reduced pressure to give the title compound, to be used without further purification, (300 mg crude) as yellow oil. MS (ESI): mass calcd. for C₂₉H₃₄FN₅O₂, 503.3; m/z found, 504.3 [M+H]⁺.

Step B. 4-Ethyl-1-(7-fluoro-4-isopropyl-2-(piperidin-1-yl)quinolin-6-yl)-3-(hydroxymethyl)-1H-1,2,4-triazol-5 (4H)-one. BCl₃ (1 M solution in toluene, 2.7 mL, 2.7 mmol) was added to 3-((benzyloxy)methyl)-4-ethyl-1-(7-fluoro-4-isopropyl-2-(piperidin-1-yl)quinolin-6-yl)-1H-1,2,4-triazol-5 (4H)-one (300 mg crude) in DCM (8 mL) at −78° C. under N₂. The reaction mixture was stirred at −78° C. for 1 hour. The reaction mixture was quenched with MeOH (2 mL) at −78° C. and stirred for 0.5 hour. After 0.5 hours, the reaction mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 45-75% B in A over 9 min, flow rate: 60 mL/min) to give the crude compound (50 mg). Further purification by preparative reversed phase HPLC (Stationary phase: Phenomenex Gemini NX-C18, 3 μm, 75×30 mm; Mobile phase: water (0.05% NH₃H₂O+10 mM NH₄HCO₃) (A) —MeCN (B), gradient elution: 40-70% B in A over 6 min, flow rate: 25 mL/min) afforded the title compound (35 mg, 84.27 μmol, 39% yield of two steps) as white powder. MS (ESI): mass calcd. for C₂₂H₂₈FN₅O₂, 413.2; m/z found, 414.1 [M+H]⁺. ¹H NMR (400 MHz, CDCl₃) δ=7.87 (d, J=8.3 Hz, 1H), 7.36 (d, J=12.3 Hz, 1H), 6.76 (s, 1H), 4.57 (s, 2H), 3.83 (q, J=7.2 Hz, 2H), 3.66 (br s, 4H), 3.43 (spt, J=6.8 Hz, 1H), 2.44 (br s, 1H), 1.62 (br s, 6H), 1.34 (t, J=7.2 Hz, 3H), 1.27 (d, J=6.8 Hz, 6H); ¹⁹F NMR (376 MHz, CDCl₃) δ=−121.44 (br s, 1F).

### Example 15: 1-(2-(3-Cyclopropyl-1H-pyrazol-4-yl)-7-fluoro-4-isopropylquinolin-6-yl)-4-ethyl-3-(hydroxymethyl)-1H-1,2,4-triazol-5 (4H)-one

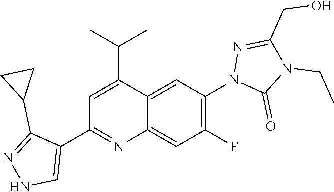 [SMILES: CCn1c(CO)nn(-c2cc3c(C(C)C)cc(-c4c[nH]nc4C4CC4)nc3cc2F)c1=O]

The title compound was prepared in a manner analogous to Example 1, Steps A-B, except using 1-cyclopropyl-4-(4,4,5,5-tetramethyl-1,3,2-dioxaborolan-2-yl)-1H-pyrazole instead of (2-methoxyphenyl)boronic acid in Step A. MS (ESI): mass calcd. for C₂₃H₂₅FN₆O₂, 436.2; m/z found, 437.1 [M+H]⁺. ¹H NMR (400 MHz, CDCl₃) δ=8.28 (d, J=8.1 Hz, 1H), 8.14 (s, 1H), 7.86 (d, J=11.8 Hz, 1H), 7.77 (s, 1H), 4.73 (s, 2H), 3.97 (q, J=7.3 Hz, 2H), 3.70 (td, J=6.8, 13.7 Hz, 1H), 2.62-2.52 (m, 1H), 1.50-1.42 (m, 9H), 1.15-1.09 (m, 2H), 0.97-0.92 (m, 2H); ¹⁹F NMR (376 MHz, CDCl₃) δ=−119.73 (br s, 1F).

### Example 16: 4-Ethyl-2-(7-fluoro-4-isopropyl-2-(3-methyl-1H-pyrazol-4-yl)quinolin-6-yl)-5-(hydroxymethyl)-2,4-dihydro-3H-1,2,4-triazol-3-one

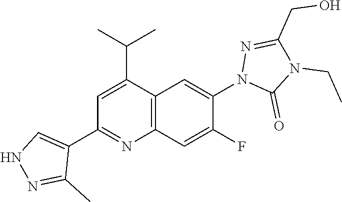 [SMILES: CCn1c(CO)nn(-c2cc3c(C(C)C)cc(-c4c[nH]nc4C)nc3cc2F)c1=O]

The title compound was prepared in a manner analogous to Example 2, Steps A-B, except using 3-methyl-4-(4,4,5,5-tetramethyl-1,3,2-dioxaborolan-2-yl)-1H-pyrazole instead of 3-chloro-5-methyl-4-(4,4,5,5-tetramethyl-1,3,2-dioxaborolan-2-yl)-1H-pyrazole (Intermediate 3) in Step A. MS (ESI): mass calcd. for C₂₁H₂₃FN₆O₂, 410.2; m/z found, 411.1 [M+H]⁺. ¹H NMR (400 MHz, DMSO-d₆) δ=13.03-12.77 (m, 1H), 8.62-8.17 (m, 2H), 7.83 (d, J=11.5 Hz, 1H), 7.71 (s, 1H), 5.82 (t, J=5.8 Hz, 1H), 4.50 (d, J=5.5 Hz, 2H), 3.82 (q, J=7.0 Hz, 2H), 3.67 (td, J=6.9, 13.6 Hz, 1H), 2.69 (br s, 3H), 1.38 (d, J=6.8 Hz, 6H), 1.31 (t, J=7.2 Hz, 3H); ¹⁹F NMR (376 MHz, DMSO-d₆) δ=−119.58 (s, 1F).

### Example 17: (S)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one

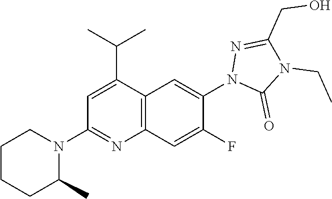 [SMILES: CCn1c(CO)nn(-c2cc3c(C(C)C)cc(N4CCCC[C@@H]4C)nc3cc2F)c1=O]

Step A. (S)-3-((Benzyloxy)methyl)-4-ethyl-1-(7-fluoro-4-isopropyl-2-(2-methylpiperidin-1-yl)quinolin-6-yl)-1H-1,2,4-triazol-5 (4H)-one. PEPPSI-iPr (3 mg, 4.40 μmol) was added to a solution of 3-((benzyloxy)methyl)-1-(2-chloro-7-fluoro-4-isopropylquinolin-6-yl)-4-ethyl-1H-1,2,4-triazol-5 (4H)-one (Intermediate 2, 200 mg, 439.6 μmol), (S)-2-methylpiperidine (87.2 mg, 879.3 μmol) and tBuONa (169 mg, 1.76 mmol) in dioxane (2.4 mL) under N₂. The reaction mixture was heated at 100° C. overnight. The reaction mixture was diluted with H₂O (20 mL) and extracted with ethyl acetate (25 mL×3). The organic layer was dried over Na₂SO₄, filtered, and concentrated under reduced pressure. Purification (FCC, SiO₂, 0-50% ethyl acetate in petroleum ether) afforded the title compound (73 mg, 123.0 μmol, 28% yield) as yellow oil. MS (ESI): mass calcd. for C₃₀H₃₆FN₅O₂, 517.3; m/z found, 518.1 [M+H]⁺. ¹H NMR (400 MHz, CDCl₃) δ=7.87 (d, J=8.2 Hz, 1H), 7.35 (d, J=12.3 Hz, 1H), 7.32-7.24 (m, 5H), 6.75 (s, 1H), 4.83-4.72 (m, 1H), 4.55 (s, 2H), 4.46 (s, 2H), 4.40 (d, J=15.0 Hz, 1H), 3.81 (q, J=7.2 Hz, 2H), 3.44 (td, J=6.8, 13.6 Hz, 1H), 2.96 (dt, J=2.9, 13.1 Hz, 1H), 1.82-1.71 (m, 2H), 1.70-1.57 (m, 3H), 1.48-1.42 (m, 1H), 1.32-1.26 (m, 9H), 1.15 (d, J=6.8 Hz, 3H); ¹⁹F NMR (376 MHz, CDCl₃) δ=−121.52 (s, 1F).

Step B. (S)-4-Ethyl-1-(7-fluoro-4-isopropyl-2-(2-methylpiperidin-1-yl)quinolin-6-yl)-3-(hydroxymethyl)-1H-1,2,4-triazol-5 (4H)-one. BCl₃ (1 M solution in toluene, 0.85 mL, 0.85 mmol) was added to (S)-3-((benzyloxy) methyl)-4-ethyl-1-(7-fluoro-4-isopropyl-2-(2-methylpiperidin-1-yl)quinolin-6-yl)-1H-1,2,4-triazol-5 (4H)-one (88 mg, 170.0 μmol) in DCM (3 mL) at −78° C. under N₂. The reaction mixture was stirred at −78° C. for 1 hour. The reaction mixture was quenched with MeOH (1.5 mL) at −78° C., and stirred at −78° C. for 0.5 hour. The reaction mixture was diluted with DCM (20 mL), and washed with sat. aq. NaHCO₃ (15 mL). The combined organic phase was dried with anhydrous Na₂SO₄, filtered, and concentrated under reduced pressure. Purification (FCC, SiO₂, 0-90% ethyl acetate in petroleum ether), then further purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 65-95% B in A over 7 min, flow rate: 25 mL/min) afforded the title compound (35 mg, 80.2 μmol, 47% yield) as white powder. MS (ESI): mass calcd. for C₂₃H₃₀FN₅O₂, 427.2; m/z found, 428.1 [M+H]⁺. ¹H NMR (400 MHz, CDCl₃) δ=7.87 (d, J=8.3 Hz, 1H), 7.35 (d, J=12.4 Hz, 1H), 6.75 (s, 1H), 4.82-4.73 (m, 1H), 4.60 (s, 2H), 4.40 (d, J=12.6 Hz, 1H), 3.84 (q, J=7.3 Hz, 2H), 3.49-3.37 (m, 1H), 2.96 (dt, J=2.9, 13.1 Hz, 1H), 2.09 (s, 1H), 1.82-1.70 (m, 2H), 1.69-1.57 (m, 3H), 1.48-1.41 (m, 1H), 1.36 (t, J=7.2 Hz, 3H), 1.28 (d, J=6.8 Hz, 6H), 1.15 (d, J=6.9 Hz, 3H); ¹⁹F NMR (376 MHz, CDCl₃) δ=−121.66 (s, 1F).

### Example 18: (R)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one

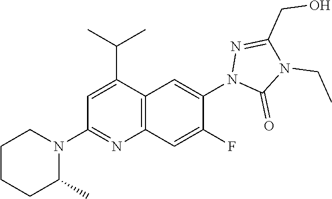 [SMILES: CCn1c(CO)nn(-c2cc3c(C(C)C)cc(N4CCCC[C@H]4C)nc3cc2F)c1=O]

The title compound was prepared in a manner analogous to Example 17, Steps A-B, except using (R)-2-methylpiperidine instead of (S)-2-methylpiperidine in Step A. MS (ESI): mass calcd. for C₂₃H₃₀FN₅O₂, 427.2; m/z found, 428.1 [M+H]⁺. ¹H NMR (400 MHz, CDCl₃) δ=7.95 (d, J=8.3 Hz, 1H), 7.43 (d, J=12.3 Hz, 1H), 6.83 (s, 1H), 4.85 (t, J=5.8 Hz, 1H), 4.67 (d, J=4.1 Hz, 2H), 4.48 (br d, J=12.6 Hz, 1H), 3.92 (q, J=7.3 Hz, 2H), 3.51 (spt, J=6.9 Hz, 1H), 3.04 (dt, J=2.9, 13.1 Hz, 1H), 2.20 (br s, 1H), 1.91-1.77 (m, 2H), 1.77-1.64 (m, 3H), 1.57-1.49 (m, 1H), 1.43 (t, J=7.2 Hz, 3H), 1.35 (d, J=6.8 Hz, 6H), 1.23 (d, J=6.9 Hz, 3H); ¹⁹F NMR (376 MHz, CDCl₃) δ=−121.65 (s, 1F).

### Example 19: 4-Ethyl-2-(7-fluoro-2-(2-hydroxy-3,5-dimethylpyridin-4-yl)-4-isopropylauinolin-6-yl)-5-(hydroxymethyl)-2,4-dihydro-3H-1,2,4-triazol-3-one

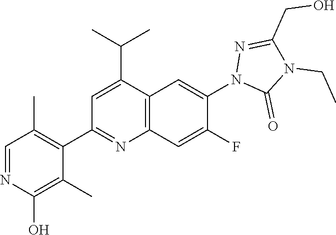 [SMILES: CCn1c(CO)nn(-c2cc3c(C(C)C)cc(-c4c(C)cnc(O)c4C)nc3cc2F)c1=O]

To a solution of 3-((benzyloxy)methyl)-4-ethyl-1-(7-fluoro-4-isopropyl-2-(2-methoxy-3,5-dimethylpyridin-4-yl)quinolin-6-yl)-1H-1,2,4-triazol-5 (4H)-one (160 mg, 287.95 μmol, Example 4, Product from Step A) dissolved in dioxane (3 mL) was added conc. HCl (1.6 mL). The reaction mixture was stirred at 100° C. for 8 hours. The reaction mixture was concentrated under reduced pressure. The resulting residue was diluted with water (1 mL) and ethyl acetate (1 mL). Sat. aq. NaHCO₃ (5 mL) was added to the reaction mixture to adjust pH to 7-8. The reaction mixture was extracted with ethyl acetate (10 mL×2). The organic layers were separated, combined, dried over Na₂SO₄, filtered, and concentrated under reduced pressure. The residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 25-55% B in A over 7 min, flow rate: 25 mL/min) to give the title compound (72.5 mg, 160.6 μmol, 56% yield) as white powder. MS (ESI): mass calcd. for C₂₄H₂₆FN₅O₃, 451.2; m/z found, 452.1 [M+H]⁺. ¹H NMR (400 MHz, CDCl₃) δ=12.21 (br s, 1H), 8.40 (d, J=8.0 Hz, 1H), 7.94 (d, J=11.3 Hz, 1H), 7.25 (s, 1H), 7.17 (s, 1H), 4.73 (s, 2H), 3.97 (q, J=7.3 Hz, 2H), 3.76 (spt, J=6.8 Hz, 1H), 2.87 (br s, 1H), 1.95 (s, 3H), 1.78 (s, 3H), 1.51-1.39 (m, 9H); ¹⁹F NMR (376 MHz, CDCl₃) δ=−117.74 (s, 1F).

### Example 20: 4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-ene

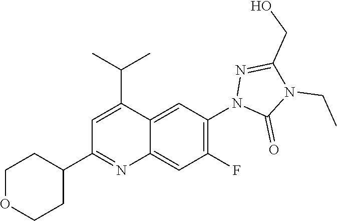 [SMILES: CCn1c(CO)nn(-c2cc3c(C(C)C)cc(C4CCOCC4)nc3cc2F)c1=O]

Step A. 3-((Benzyloxy)methyl)-4-ethyl-1-(7-fluoro-4-isopropyl-2-(tetrahydro-2H-pyran-4-yl)quinolin-6-yl)-1H-1,2,4-triazol-5 (4H)-one. To a mixture of 3-((benzyloxy)methyl)-1-(2-chloro-7-fluoro-4-isopropylquinolin-6-yl)-4-ethyl-1H-1,2,4-triazol-5 (4H)-one (Intermediate 2, 250 mg, 549.54 μmol) in DMA (5 mL) was added 2-dicyclohexylphosphino-2′,6′-bis(N,N-dimethylamino)biphenyl (Cphos) (24 mg, 54.97 μmol) and Pd(OAc)₂ (12.3 mg, 54.97 μmol). The reaction mixture was charged with N₂. Tetrahydropyran-4-ylzinc bromide (0.5 M solution in THF, 16.5 mL, 8.25 mmol) was added to the reaction mixture under N₂. The reaction mixture was stirred at 35° C. for 16 hours, then cooled to room temperature. The reaction mixture was diluted with water (15 mL) and extracted with ethyl acetate (20 mL×3). The combined organic layers were combined, dried over Na₂SO₄, filtered, and concentrated under reduced pressure. The residue was purified by flash column chromatography (SiO₂, gradient elution: 0-20% ethyl acetate in petroleum ether) to give the title compound (140 mg, 259 μmol, 47% yield) as a yellow oil. MS (ESI): mass calcd. for C₂₉H₃₃FN₄O₃, 504.3; m/z found, 505.2 [M+H]⁺. ¹H NMR (400 MHz, CDCl₃) δ=8.26 (d, J=8.1 Hz, 1H), 7.84 (d, J=11.6 Hz, 1H), 7.43-7.32 (m, 5H), 7.24 (s, 1H), 4.64 (s, 2H), 4.24-4.09 (m, 2H), 3.90 (q, J=7.3 Hz, 2H), 3.81-3.56 (m, 4H), 3.50-3.40 (m, 1H), 3.14 (tt, J=3.8, 11.8 Hz, 1H), 1.98-1.83 (m, 4H), 1.46-1.36 (m, 9H); ¹⁹F NMR (376 MHz, CDCl₃) δ=−119.33 (s, 1F).

Step B. 4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-one. To a solution of 3-((benzyloxy)methyl)-4-ethyl-1-(7-fluoro-4-isopropyl-2-(tetrahydro-2H-pyran-4-yl)quinolin-6-yl)-1H-1,2,4-triazol-5 (4H)-one (140 mg, 258.67 μmol) dissolved in DCM (4 mL) was added BCl₃ (1 M solution in toluene, 1.6 mL, 1.6 mmol) at −78° C. under N₂. The reaction mixture was stirred at −78° C. for 1 hour. The reaction mixture was quenched with MeOH (2 mL) at-78° C. and stirred for 0.5 hour. After 0.5 hours, the reaction mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The residue was purified by preparative reversed phase HPLC (Stationary phase: Phenomenex Gemini-NX, 5 μm, 150×30 mm; Mobile phase: water (0.05% NH₃H₂O) (A) —MeCN (B), gradient elution: 35-61% B in A over 6 min, flow rate: 35 mL/min) to give the title compound (48.1 mg, 116 μmol, 45% yield) as a white powder. MS (ESI): mass calcd. for C₂₂H₂₇FN₄O₃, 414.2; m/z found, 415.3 [M+H]⁺. ¹H NMR (400 MHz, CDCl₃) δ=8.28 (d, J=8.1 Hz, 1H), 7.85 (d, J=11.7 Hz, 1H), 7.25 (s, 1H), 4.72 (d, J=6.2 Hz, 2H), 4.16 (dd, J=3.4, 11.1 Hz, 2H), 3.96 (q, J=7.2 Hz, 2H), 3.77-3.57 (m, 3H), 3.15 (tt, J=3.9, 11.8 Hz, 1H), 2.36 (t, J=6.3 Hz, 1H), 2.15-1.89 (m, 4H), 1.46 (t, J=7.2 Hz, 3H), 1.41 (d, J=6.9 Hz, 6H); ¹⁹F NMR (376 MHz, CDCl₃) δ=−119.44 (s, 1F).

### Example 21: 6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide

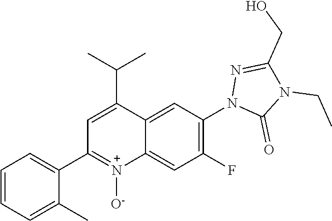 [SMILES: CCn1c(CO)nn(-c2cc3c(C(C)C)cc(-c4ccccc4C)[n+]([O-])c3cc2F)c1=O]

Step A. 6-(3-((Benzyloxy)methyl)-4-ethyl-5-oxo-4,5-dihydro-1H-1,2,4-triazol-1-yl)-7-fluoro-4-isopropyl-2-(o-tolyl)quinoline 1-oxide. mCPBA (85% purity, 99.5 mg, 489.7 μmol) was added to a solution of 3-((benzyloxy)methyl)-4-ethyl-1-(7-fluoro-4-isopropyl-2-(o-tolyl)quinolin-6-yl)-1H-1,2,4-triazol-5 (4H)-one (Example 9, product from Step A, 200 mg, 376.79 μmol) in CHCl₃ (2 mL). The reaction mixture was stirred at 70° C. overnight. The reaction mixture was cooled to room temperature and quenched with sat. aq. Na₂SO₃ (35 mL). Sat. aq. Na₂CO₃ was added to adjust pH to 9-10 and the mixture was extracted with DCM (25 mL×3). The combined organic phase was dried with anhydrous Na₂SO₄, filtered, and concentrated under reduced pressure. The crude product was purified by flash column chromatography (SiO₂, gradient elution: 0-100% ethyl acetate in petroleum ether) to give the title compound (105 mg, 197.4 μmol, 52% yield) as a yellow solid. MS (ESI): mass calcd. for C₃₁H₃₁FN₄O₃, 526.2; m/z found, 527.2 [M+H]⁺. ¹H NMR (400 MHz, CDCl₃) δ=8.77 (d, J=11.8 Hz, 1H), 8.45 (d, J=7.3 Hz, 1H), 7.49-7.33 (m, 9H), 7.26 (s, 1H), 4.67 (s, 2H), 4.58 (s, 2H), 3.93 (q, J=7.3 Hz, 2H), 3.66 (td, J=6.7, 13.5 Hz, 1H), 2.28 (s, 3H), 1.46-1.39 (m, 9H); ¹⁹F NMR (376 MHz, CDCl₃) δ=−114.96 (br s, 1F).

Step B. 6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide. BCl₃ (1 M solution in toluene, 0.99 mL, 0.99 mmol) was added to a stirred solution of 6-(3-((benzyloxy)methyl)-4-ethyl-5-oxo-4,5-dihydro-1H-1,2,4-triazol-1-yl)-7-fluoro-4-isopropyl-2-(o-tolyl)quinoline 1-oxide (105 mg, 197.38 μmol) in DCM (5 mL) at −78° C., and the reaction mixture was stirred at −78° C. for 1 hour. The reaction mixture was quenched with MeOH (2 mL) at −78° C. and stirred for 0.5 hour. After 0.5 hours, the reaction mixture was warmed to room temperature, diluted with DCM (12 mL) and washed with sat. aq. NaHCO₃ (15 mL). The combined organic phase was washed with brine (5 mL×3), dried with anhydrous Na₂SO₄, filtered, and concentrated under reduced pressure. The crude product was purified twice by preparative reversed phase HPLC (The first: Stationary phase: Phenomenex Gemini-NX, 5 μm, 150×30 mm; Mobile phase: water (0.05% NH₃H₂O) (A) —MeCN (B), gradient elution: 27-57% B in A over 7 min, flow rate: 25 mL/min; The second: Stationary phase: Boston Prime C18, 5 μm, 150×30 mm; Mobile phase: water (0.05% NH₃H₂O+10 mM NH₄HCO₃) (A) —MeCN (B), gradient elution: 35-65% B in A over 7 min, flow rate: 25 mL/min) to give the title compound (45 mg, 103.1 μmol, 52% yield) as a white powder. MS (ESI): mass calcd. For C₂₄H₂₅FN₄O₃, 436.2; m/z found, 437.1 [M+H]⁺. ¹H NMR (400 MHz, CDCl₃) δ=8.74 (d, J=11.8 Hz, 1H), 8.44 (d, J=7.3 Hz, 1H), 7.45-7.40 (m, 1H), 7.39-7.34 (m, 3H), 7.25 (s, 1H), 4.70 (d, J=6.3 Hz, 2H), 4.00-3.90 (m, 2H), 3.64 (quin, J=6.8 Hz, 1H), 2.41 (t, J=6.4 Hz, 1H), 2.27 (s, 3H), 1.48-1.40 (m, 9H); ¹⁹F NMR (376 MHz, CDCl₃) δ=−114.86-114.96 (m, 1F).

### Example 22: 4-Ethyl-1-(7-fluoro-2-(2-hydroxy-3-methylpyridin-4-yl)-4-isopropylquinolin-6-yl)-3-(hydroxymethyl)-1H-1,2,4-triazol-5 (4H)-one

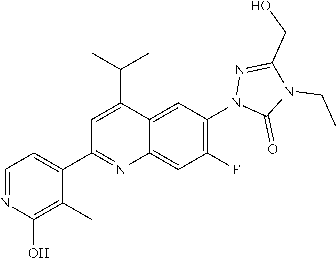 [SMILES: CCn1c(CO)nn(-c2cc3c(C(C)C)cc(-c4ccnc(O)c4C)nc3cc2F)c1=O]

The title compound was prepared in a manner analogous to Example 1, Steps A-B, except using 3-methyl-4-(4,4,5,5-tetramethyl-1,3,2-dioxaborolan-2-yl)pyridin-2-ol (Intermediate 11) instead of (2-methoxyphenyl)boronic acid in Step A. MS (ESI): mass calcd. for C₂₃H₂₄FN₅O₃, 437.2; m/z found, 438.1 [M+H]⁺. ¹H NMR (400 MHz, CDCl₃) δ=12.29 (br s, 1H), 8.41 (d, J=8.0 Hz, 1H), 7.96 (d, J=11.3 Hz, 1H), 7.43 (s, 1H), 7.40 (d, J=6.5 Hz, 1H), 6.52 (d, J=6.8 Hz, 1H), 4.74 (s, 2H), 3.98 (q, J=7.2 Hz, 2H), 3.76 (td, J=6.8, 13.6 Hz, 1H), 2.76 (br s, 1H), 2.22 (s, 3H), 1.52-1.42 (m, 9H); ¹⁹F NMR (376 MHz, CDCl₃) δ=−117.78 (s, 1F).

### Example 23: 4-Ethyl-1-(7-fluoro-4-isopropyl-2-(3-(trifluoromethyl)-pH-pyrazol-4-yl)quinolin-6-yl)-3-(hydroxymethyl)-1H-1,2,4-triazol-5 (4H)-one

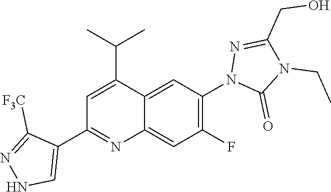 [SMILES: CCn1c(CO)nn(-c2cc3c(C(C)C)cc(-c4c[nH]nc4C(F)(F)F)nc3cc2F)c1=O]

Step A. 3-((Benzyloxy)methyl)-4-ethyl-1-(7-fluoro-4-isopropyl-2-(3-(trifluoromethyl)-1H-pyrazol-4-yl)quinolin-6-yl)-1H-1,2,4-triazol-5 (4H)-one. (3-(Trifluoromethyl)-1H-pyrazol-4-yl)boronic acid (165.2 mg, 918.1 μmol), K₂CO₃ (253.8 mg, 1.84 mmol) and Pd-118 (39.9 mg, 61.21 μmol) was added into the solution of 3-((benzyloxy)methyl)-1-(2-chloro-7-fluoro-4-isopropylquinolin-6-yl)-4-ethyl-1H-1,2,4-triazol-5 (4H)-one (Intermediate 2, 300 mg, 612.1 μmol) in dioxane/H₂O (v/v, 5/1, 7.2 mL) at room temperature under N₂. The reaction mixture was stirred at 80° C. for 5 hours. The reaction mixture was diluted with ethyl acetate (30 mL) and H₂O (20 mL). The organic layer was separated, and the aqueous layer was extracted with ethyl acetate (30 mL×3). The combined organic layers were dried over Na₂SO₄, filtered, and concentrated under reduced pressure. The crude product was purified by column chromatography (SiO₂, gradient elution: 0-90% ethyl acetate in petroleum ether) to give the title compound (190 mg, 323.7 μmol, 52.9% yield) as yellow solid. MS (ESI): mass calcd. for C₂₈H₂₆F₄N₆O₂, 554.2; m/z found, 555.1 [M+H]⁺. ¹H NMR (400 MHz, CDCl₃) δ=11.96 (br s, 1H), 8.21 (d, J=8.0 Hz, 1H), 7.88 (d, J=11.3 Hz, 1H), 7.81 (s, 1H), 7.51 (s, 1H), 7.44-7.31 (m, 5H), 4.66 (s, 2H), 4.58 (s, 2H), 3.93 (q, J=7.2 Hz, 2H), 3.58 (td, J=6.7, 13.5 Hz, 1H), 1.43 (br t, J=7.2 Hz, 3H), 1.37 (d, J=6.8 Hz, 6H); ¹⁹F NMR (376 MHz, CDCl₃) δ=−59.76 (s, 1F), −118.84 (br s, 1F).

Step B. 4-Ethyl-1-(7-fluoro-4-isopropyl-2-(3-(trifluoromethyl)-1H-pyrazol-4-yl)quinolin-6-yl)-3-(hydroxymethyl)-1H-1,2,4-triazol-5 (4H)-one. BCl₃ (1 M solution in toluene, 0.8 mL, 0.8 mmol) was added to 3-((benzyloxy)methyl)-4-ethyl-1-(7-fluoro-4-isopropyl-2-(3-(trifluoromethyl)-1H-pyrazol-4-yl)quinolin-6-yl)-1H-1,2,4-triazol-5 (4H)-one (90 mg, 162.30 μmol) in DCM (5 mL) at −78° C. under N₂. The reaction mixture was stirred at −78° C. for 1 hour. The reaction mixture was quenched with MeOH (1.5 mL) at −78° C., and stirred at −78° C. for 0.5 hour. The reaction mixture were diluted with DCM (20 mL), washed with sat. aq. NaHCO₃ (18 mL). The organic phase was dried with anhydrous Na₂SO₄, filtered, and concentrated under reduced pressure. The resulting residue was purified by preparative reversed phase HPLC (Stationary phase: Boston Prime C18, 5 μm, 150×30 mm; Mobile phase: water (0.05% NH₃H₂O+10 mM NH₄HCO₃) (A) —MeCN (B), gradient elution: 40-70% B in A over 7 min, flow rate: 25 mL/min) to give the title compound (37 mg, 79.67 μmol, 49.09% yield, 100% purity) as white powder. MS (ESI): mass calcd. for C₂₁H₂₀F₄N₆O₂, 464.2 m/z found, 464.9 [M+H]⁺. ¹H NMR (400 MHz, DMSO-d₆) δ=8.78 (s, 1H), 8.36 (d, J=8.2 Hz, 1H), 7.81 (d, J=11.4 Hz, 1H), 7.75 (s, 1H), 4.50 (s, 2H), 3.82 (q, J=7.1 Hz, 2H), 3.77-3.67 (m, 1H), 1.37 (d, J=6.8 Hz, 6H), 1.31 (t, J=7.1 Hz, 3H); ¹⁹F NMR (376 MHz, DMSO-d₆) δ=−57.98-59.41 (m, 1F), −118.65 (br s, 1F).

### Example 24: 4-Ethyl-1-(7-fluoro-2-(2-hydroxy-5-methylpyridin-4-yl)-4-isopropylquinolin-6-yl)-3-(hydroxymethyl)-1H-1,2,4-triazol-5 (4H)-one

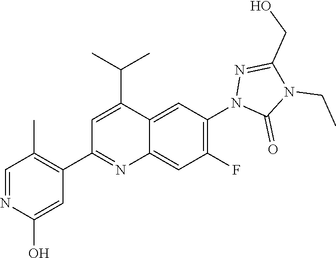 [SMILES: CCn1c(CO)nn(-c2cc3c(C(C)C)cc(-c4cc(O)ncc4C)nc3cc2F)c1=O]

The title compound was prepared in a manner analogous to Example 1, Steps A-B, except using (benzyloxy)-5-methyl-4-(4,4,5,5-tetramethyl-1,3,2-dioxaborolan-2-yl)pyridine (Intermediate 12, 120.7 mg, 336.77 μmol) instead of (2-methoxyphenyl)boronic acid in Step A. MS (ESI): mass calcd. for C₂₃H₂₄FN₅O₃, 437.2; m/z found, 438.0 [M+H]⁺. ¹H NMR (400 MHz, DMSO-d₆) δ=8.45 (d, J=8.0 Hz, 1H), 8.03 (d, J=11.5 Hz, 1H), 7.61 (s, 1H), 7.36 (s, 1H), 6.52 (s, 1H), 5.84 (br s, 1H), 4.52 (s, 2H), 3.83 (q, J=7.3 Hz, 2H), 3.78-3.72 (m, 1H), 2.03 (s, 3H), 1.38 (d, J=6.8 Hz, 6H), 1.32 (t, J=7.2 Hz, 3H); ¹⁹F NMR (376 MHz, DMSO-d₆) δ=−118.16 (s, 1F).

### Example 25: 2-(2-(4-Chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one

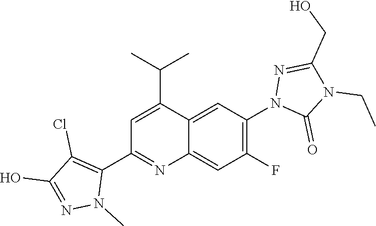 [SMILES: CCn1c(CO)nn(-c2cc3c(C(C)C)cc(-c4c(Cl)c(O)nn4C)nc3cc2F)c1=O]

Step A. 3-((Benzyloxy)methyl)-4-ethyl-1-(7-fluoro-4-isopropyl-2-(3-methoxy-1-methyl-1H-pyrazol-5-yl)quinolin-6-yl)-1H-1,2,4-triazol-5 (4H)-one. A mixture of 3-methoxy-1-methyl-5-(4,4,5,5-tetramethyl-1,3,2-dioxaborolan-2-yl)-1H-pyrazole (Intermediate 13, 340.03 mg, 1.42 mmol), 3-((benzyloxy)methyl)-1-(2-chloro-7-fluoro-4-isopropylquinolin-6-yl)-4-ethyl-1H-1,2,4-triazol-5 (4H)-one (Intermediate 2, 350 mg, 714.07 μmol), Pd-118 (47 mg, 72.11 μmol) and K₂CO₃ (296 mg, 2.14 mmol) in dioxane/H₂O (v/v, 5/1, 10 mL) was bubbled with N₂ for 2 minutes. The reaction mixture was stirred at 90° C. for 16 hours, then cooled to room temperature. The reaction mixture was filtered, and the filtrate was extracted with ethyl acetate (50 mL×3). The combined organic layers were washed with brine (15 mL), dried over Na₂SO₄, filtered, and concentrated under reduced pressure. The residue was purified by flash column chromatography (SiO₂, gradient elution: 0-60% ethyl acetate in petroleum ether) to give the title compound (360 mg, 610.39 μmol, 85% yield) as a yellow oil. MS (ESI): mass calcd. for C₂₉H₃₁FN₆O₃, 530.2; m/z found, 531.2 [M+H]⁺. ¹H NMR (400 MHz, CDCl₃) δ=8.33 (d, J=7.8 Hz, 1H), 7.91 (d, J=11.3 Hz, 1H), 7.54 (s, 1H), 7.46-7.33 (m, 5H), 6.17 (s, 1H), 4.66 (s, 2H), 4.58 (s, 2H), 4.24 (s, 3H), 3.97 (s, 3H), 3.93-3.89 (m, 2H), 3.73-3.65 (m, 1H), 1.95 (s, 3H), 1.49-1.43 (m, 6H); ¹⁹F NMR (376 MHz, CDCl₃) δ=−118.22 (s, 1F).

Step B. 3-((Benzyloxy)methyl)-1-(2-(4-chloro-3-methoxy-1-methyl-1H-pyrazol-5-yl)-7-fluoro-4-isopropylquinolin-6-yl-4-ethyl-1H-1,2,4-triazol-5 (4H)-one. 1-Chloropyrrolidine-2,5-dione (158 mg, 1.19 mmol) was slowly added into a solution of 3-((benzyloxy)methyl)-4-ethyl-1-(7-fluoro-4-isopropyl-2-(3-methoxy-1-methyl-1H-pyrazol-5-yl)quinolin-6-yl)-1H-1,2,4-triazol-5 (4H)-one (350 mg, 593.44 μmol) in MeCN (6 mL) at room temperature. The reaction mixture was heated to 45° C. for 16 hours. The reaction mixture was cooled, concentrated, and the residue was dissolved into the ethyl acetate (50 mL) and water (20 mL). The organic layer was separated, washed with brine (20 mL), dried over Na₂SO₄, filtered, and concentrated under reduced pressure. The residue was purified by flash column chromatography (SiO₂, gradient elution: 0-100% ethyl acetate in petroleum ether) to give the title compound (200 mg, 344.6 μmol, 58% yield) as a yellow oil. MS (ESI): mass calcd. for C₂₉H₃₀ClFN₆O₃, 564.2; m/z found, 565.1 [M+H]⁺. ¹H NMR (400 MHz, CDCl₃) δ=8.38 (d, J=8.0 Hz, 1H), 7.93 (d, J=11.3 Hz, 1H), 7.85 (s, 1H), 7.45-7.34 (m, 5H), 4.67 (s, 2H), 4.58 (s, 2H), 4.08 (s, 3H), 4.06 (s, 3H), 3.97-3.88 (m, 2H), 3.75 (quin, J=6.9 Hz, 1H), 1.47 (d, J=6.9 Hz, 6H), 1.42 (t, J=7.2 Hz, 3H); ¹⁹F NMR (376 MHz, CDCl₃) δ=−117.79 (s, 1F).

Step C. 2-(2-(4-Chloro-3-hydroxy-1-methyl-H-pyrazol-5-yl-7-fluoro-4-isopropylauinolin-6-yl)-4-ethyl-5-(hydroxymethyl)-2,4-dihydro-3H-1,2,4-triazol-3-one. To a solution of 3-((benzyloxy)methyl)-1-(2-(4-chloro-3-methoxy-1-methyl-1H-pyrazol-5-yl)-7-fluoro-4-isopropylquinolin-6-yl)-4-ethyl-1H-1,2,4-triazol-5 (4H)-one (190 mg, 327.38 μmol) in DCM (5 mL) was added BBr₃ (1 M solution in toluene, 3.27 mL, 3.27 mmol) at −30° C. The reaction mixture was stirred at −30° C. for 30 minutes and then stirred at room temperature for 5 hours under N₂. The reaction mixture was quenched with MeOH (2 mL) at −78° C., stirred at same temperature for 0.5 hour. The reaction mixture was warmed to room temperature, was diluted with DCM (20 mL) and washed with sat. aq. NaHCO₃ (12 mL). The organic layers were washed with brine (10 mL), dried over Na₂SO₄, filtered, and concentrated under reduced pressure. The resulting residue was purified by preparative reversed phase HPLC (Stationary phase: Boston Prime C18, 5 μm, 150×30 mm; Mobile phase: water (0.04% NH₃H₂O+10 mM NH₄HCO₃) (A) —MeCN (B), gradient elution: 15-45% B in A over 7 min, flow rate: 25 mL/min) to give the title compound (80 mg, 173.58 μmol, 53% yield) as an off-white powder. MS (ESI): mass calcd. for C₂₁H₂₂ClFN₆O₃, 460.1; m/z found, 461.1 [M+H]⁺. ¹H NMR (400 MHz, MeOD) δ=8.47 (d, J=7.9 Hz, 1H), 7.96 (d, J=11.2 Hz, 1H), 7.90 (s, 1H), 4.66 (s, 2H), 3.98 (q, J=7.1 Hz, 2H), 3.91 (s, 3H), 3.83 (td, J=6.8, 13.5 Hz, 1H), 1.50-1.46 (m, 6H), 1.46-1.42 (m, 3H); ¹⁹F NMR (376 MHz, MeOD) δ=−119.54 (s, 1F).

### Example 26: 2-(2-(4-Chloro-3-methoxy-1-methyl-1H-pyrazol-5-yl)-7-fluoro-4-isopropylauinolin-6-yl)-4-ethyl-5-(hydroxymethyl)-2,4-dihydro-3H-1,2,4-triazol-3-one

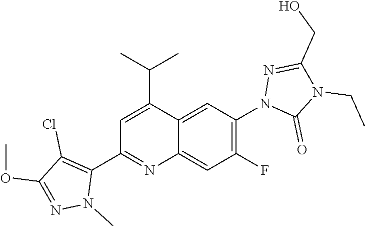 [SMILES: CCn1c(CO)nn(-c2cc3c(C(C)C)cc(-c4c(Cl)c(OC)nn4C)nc3cc2F)c1=O]

To a solution of 2-(2-(4-chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one (Example 25, 55 mg, 119.33 μmol) and Cs₂CO₃ (58 mg, 178.01 μmol) in DMF (4 mL) was added iodomethane (100 mg, 704.52 μmol) in DMF (1 mL). The reaction mixture was stirred at 30° C. for 2 hours. The reaction mixture was filtered, diluted with ethyl acetate (30 mL) and water (15 mL), then separated. The organic layer was washed with brine (10 mL), dried over Na₂SO₄, filtered, and concentrated under reduced pressure. The residue was purified by preparative reversed phase HPLC (Stationary phase: Phenomenex Gemini-NX C18, 5 μm, 150×30 mm; Mobile phase: water (0.05% NH₃H₂O) (A) —MeCN (B), gradient elution: 37-67% B in A over 7 min, flow rate: 35 mL/min) to give the title compound (30 mg, 63.14 μmol, 53% yield) as a light yellow powder. MS (ESI): mass calcd. for C₂₂H₂₄ClFN₆O₃, 474.2; m/z found, 475.2 [M+H]⁺. ¹H NMR (400 MHz, CDCl₃) δ=8.29 (d, J=8.0 Hz, 1H), 7.84 (d, J=11.4 Hz, 1H), 7.76 (s, 1H), 4.65 (br d, J=4.6 Hz, 2H), 3.99 (s, 3H), 3.97 (s, 3H), 3.88 (q, J=7.2 Hz, 2H), 3.73-3.56 (m, 1H), 2.13 (br s, 1H), 1.43-1.32 (m, 9H); ¹⁹F NMR (376 MHz, CDCl₃) δ=−117.93 (s, 1F).

### Example 27: 1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one

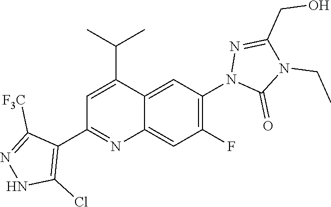 [SMILES: CCn1c(CO)nn(-c2cc3c(C(C)C)cc(-c4c(C(F)(F)F)n[nH]c4Cl)nc3cc2F)c1=O]

Step A. 3-((Benzyloxy)methyl)-1-(2-(5-chloro-3-(trifluoromethyl)-1H-pyrazol-4-yl)-7-fluoro-4-isopropylquinolin-6-yl)-4-ethyl-1H-1,2,4-triazol-5 (4H)-one. 1-chloropyrrolidine-2,5-dione (89 mg, 666.50 μmol) was slowly added into the solution of 3-((benzyloxy)methyl)-4-ethyl-1-(7-fluoro-4-isopropyl-2-(3-(trifluoromethyl)-1H-pyrazol-4-yl)quinolin-6-yl)-1H-1,2,4-triazol-5 (4H)-one (Example 23 product from Step A, 200 mg, 335 μmol) in MeCN (10 mL) at room temperature. The reaction mixture was heated to 60° C. for 16 hours. The reaction mixture was concentrated under reduced pressure and the residue was dissolved in ethyl acetate (20 mL) and water (10 mL). The mixture was separated, and the organic layer was washed with brine (10 mL), dried over Na₂SO₄, filtered, and concentrated under reduced pressure. The resulting residue was purified by flash column chromatography (SiO₂, gradient elution: 0-100% ethyl acetate in petroleum ether) to give the title compound (170 mg, 178.59 μmol, 53% yield) as a yellow oil. MS (ESI): mass calcd. for C₂₈H₂₅ClF₄N₆O₂, 588.2; m/z found, 589.1 [M+H]⁺. ¹H NMR (400 MHz, CDCl₃) δ=8.25 (d, J=7.9 Hz, 1H), 7.97 (s, 1H), 7.86 (d, J=11.3 Hz, 1H), 7.50 (s, 1H), 7.34-7.27 (m, 5H), 4.57 (s, 2H), 4.49 (s, 2H), 3.85 (q, J=7.2 Hz, 2H), 3.67-3.57 (m, 1H), 1.35 (d, J=6.9 Hz, 9H); ¹⁹F NMR (376 MHz, CDCl₃) δ=−60.25 (br s, 1F), −118.04 (br s, 1F).

Step B. 1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one. To a solution of 3-((benzyloxy)methyl)-1-(2-(5-chloro-3-(trifluoromethyl)-1H-pyrazol-4-yl)-7-fluoro-4-isopropylquinolin-6-yl)-4-ethyl-1H-1,2,4-triazol-5 (4H)-one (170 mg, 178.59 μmol) in DCM (5 mL) was added BCl₃ (1 M solution in toluene, 1.07 mL, 1.07 mmol) at −78° C. for 1 hour under N₂. The reaction mixture was quenched by addition of sat. aq. NaHCO₃ (10 mL) at 0° C., and then extracted with DCM/MeOH (v/v, 10/1, 20 mL×2). The combined organic layers were washed with brine (10 mL), dried over Na₂SO₄, filtered, and concentrated under reduced pressure. The resulting residue was purified by prep-HPLC (Stationary phase: Boston Green ODS, 5 μm, 150×30 mm; Mobile phase: water (0.2% FA) (A) —MeCN (B), gradient elution: 38-68% B in A over 7 min, flow rate: 25 mL/min) to give the title compound (45 mg, 90.20 μmol, 51% yield) as a white powder. MS (ESI): mass calcd. for C₂₁H₁₉ClF₄N₆O₂, 498.1; m/z found, 499.0 [M+H]⁺. ¹H NMR (400 MHz, DMSO-d₆) δ=14.94 (br s, 1H), 8.44 (d, J=8.1 Hz, 1H), 7.92 (d, J=11.4 Hz, 1H), 7.69 (s, 1H), 5.82 (t, J=5.8 Hz, 1H), 4.52 (d, J=5.7 Hz, 2H), 3.83 (q, J=7.2 Hz, 2H), 3.80-3.72 (m, 1H), 1.37 (d, J=6.8 Hz, 6H), 1.32 (t, J=7.2 Hz, 3H); ¹⁹F NMR (376 MHz, DMSO-d₆) δ=−59.28 (br s, 1F), −117.92 (br s, 1F).

### Example 28: (3-(7-Fluoro-4-isopropyl-2-(3-methyl-5-(trifluoromethyl)-pH-pyrazol-4-yl)quinolin-6-yl)-1-methyl-1H-1,2,4-triazol-5-yl)methanol

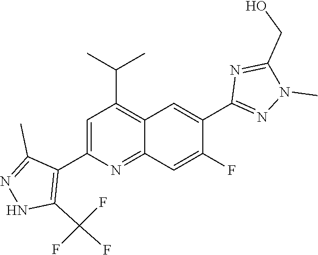 [SMILES: Cc1n[nH]c(C(F)(F)F)c1-c1cc(C(C)C)c2cc(-c3nc(CO)n(C)n3)c(F)cc2n1]

Step A: 3-(7-Fluoro-4-isopropyl-2-(3-methyl-5-(trifluoromethyl)-1H-pyrazol-4-yl)quinolin-6-yl)-N-methoxy-N,1-dimethyl-1H-1,2,4-triazole-5-carboxamide. 3-(2-Chloro-7-fluoro-4-isopropylquinolin-6-yl)-N-methoxy-N,1-dimethyl-1H-1,2,4-triazole-5-carboxamide (Intermediate 8, 160 mg, 0.41 mmol), 3-methyl-4-(4,4,5,5-tetramethyl-1,3,2-dioxaborolan-2-yl)-5-(trifluoromethyl)-1H-pyrazole (Intermediate 15, 124 mg, 0.45 mmol), XPhos Pd G3 (35 mg, 0.04 mmol), cesium carbonate (399 mg, 1.2 mmol), in dioxane/water (5:1) were purged with argon, then heated to 80° C. for 1 hour. The reaction mixture was cooled to room temperature and subsequently diluted with ethyl acetate and water. The organic layer was extracted (3×) with ethyl acetate and the combined organics were washed with brine, dried over Na₂SO₄, filtered, and concentrated under reduced pressure. The resulting residue was purified by flash column chromatography (SiO₂, eluting with a gradient of 0-60% ethyl acetate in heptane) to give the title compound (97 mg, 0.19 mmol, 47% yield) as a yellow solid. MS (ESI): mass calcd. for C₂₃H₂₃F₄N₇O₂, 505.18; m/z found, 506.3 [M+H]⁺. ¹H NMR (400 MHz, CDCl₃) δ=10.44 (br s, 1H), 8.86 (br s, 1H), 7.86 (d, J=12.23 Hz, 1H), 7.52-7.55 (m, 1H), 4.14-4.32 (m, 3H), 3.97 (br s, 3H), 3.75-3.92 (m, 2H), 3.43 (br s, 2H), 2.59-2.62 (m, 3H), 1.45 (d, J=6.85 Hz, 6H).

Step B: (3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol and 3-(7-Fluoro-4-isopropyl-2-(3-methyl-5-(trifluoromethyl)-1H-pyrazol-4-yl)quinolin-6-yl)-1-methyl-1H-1,2,4-triazole-5-carbaldehyde. To a solution of 3-(7-fluoro-4-isopropyl-2-(3-methyl-5-(trifluoromethyl)-1H-pyrazol-4-yl)quinolin-6-yl)-N-methoxy-N,1-dimethyl-1H-1,2,4-triazole-5-carboxamide in THF (1.9 mL) at −78° C. was added diisobutylaluminum hydride (1M in DCM, 0.58 mL, 0.58 mmol) and stirred at −78° C. for 10 mins. The reaction mixture was then warmed to room temperature and stirred for 2 hours. The reaction mixture was quenched with water (1 mL), 1N NaOH was added and the reaction mixture and the reaction mixture was stirred for an additional 15 minutes. The reaction mixture was extracted with ethyl acetate (3×). The combined organic layers were washed with brine, dried over Na₂SO₄, filtered, and concentrated under reduced pressure. The title compound (85 mg) was isolated as a mixture of (3-(7-fluoro-4-isopropyl-2-(3-methyl-5-(trifluoromethyl)-1H-pyrazol-4-yl)quinolin-6-yl)-1-methyl-1H-1,2,4-triazol-5-yl)methanol and (3-(7-Fluoro-4-isopropyl-2-(3-methyl-5-(trifluoromethyl)-1H-pyrazol-4-yl)quinolin-6-yl)-1-methyl-1H-1,2,4-triazole-5-carbaldehyde) and would be used in the next step without further purification. MS (ESI): mass calcd. for C₂₁H₁₈F₄N₆O, 446.15; m/z found, 447.1 [M+H]⁺.

Step C: (3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol. To a solution of the mixture of compounds from Step B (85 mg, 0.19 mmol) in MeOH/DCM (4:1) was added sodium borohydride (14 mg, 0.38 mmol) at 0° C. The reaction mixture was warmed to room temperature and stirred for 15 minutes. The reaction mixture was diluted with water and ethyl acetate. The reaction mixture was extracted with ethyl acetate (3×). The combined organic layers were washed with brine, dried over Na₂SO₄, filtered, and concentrated under reduced pressure. The crude material was purified with reverse phase chromatography (Phenomenex Gemini-NX, C18, 150×30 mm, 5 um; 30 mL/min; Buffer A: 20 mM NH₄OH/water Buffer B: MeCN; gradient: 20% B for 2 min then linear gradient to 1000% B over 12 min) to provide the title compound (15 mg, 20% yield over two steps) as a white solid. MS (ESI): mass calcd. for C₂₁H₂₀F₄N₆O₂, 448.16; m/z found, 449.2 [M+H]⁺. ¹H NMR (400 MHz, CDCl₃) δ=8.80 (d, J=7.83 Hz, 1H), 7.85 (d, J=12.23 Hz, 1H), 7.54 (s, 1H), 4.92 (s, 2H), 4.07 (s, 3H), 3.83-3.90 (m, 1H), 2.60 (s, 3H), 1.43-1.46 (m, 6H).

### Example 29: 1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-ol

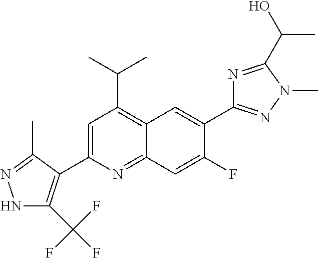 [SMILES: Cc1n[nH]c(C(F)(F)F)c1-c1cc(C(C)C)c2cc(-c3nc(C(C)O)n(C)n3)c(F)cc2n1]

Step A: 3-(7-Fluoro-4-isopropyl-2-(3-methyl-5-(trifluoromethyl)-1H-pyrazol-4-yl)quinolin-6-yl)-N-methoxy-N,1-dimethyl-1H-1,2,4-triazole-5-carboxamide. A mixture of 3-(2-chloro-7-fluoro-4-isopropylquinolin-6-yl)-N-methoxy-N,1-dimethyl-1H-1,2,4-triazole-5-carboxamide (Intermediate 8, 160 mg, 0.41 mmol), 5-methyl-4-(4,4,5,5-tetramethyl-1,3,2-dioxaborolan-2-yl)-3-(trifluoromethyl)-1H-pyrazole (Intermediate 4 124 mg, 0.45 mmol), XPhos Pd G3 (35 mg, 0.04 mmol), cesium carbonate (399 mg, 1.2 mmol), in dioxane/water (5:1) were purged with argon, then heated to 80° C. for 1 hour. The reaction mixture was cooled to room temperature and subsequently diluted with ethyl acetate and water. The organic layer was extracted (3×) with ethyl acetate and the combined organics were washed with brine, dried over Na₂SO₄, filtered, and concentrated under reduced pressure. The resulting residue was purified with flash column chromatography (SiO₂, eluting with a gradient of 0-60% ethyl acetate in heptane) to give the title compound (97 mg, 0.19 mmol, 47% yield) as a yellow solid. MS (ESI): mass calcd. for C₂₃H₂₃F₄N₇O₂, 505.18; m/z found, 506.3 [M+H]⁺. (400 MHz, CDCl₃) δ=10.44 (br s, 1H), 8.86 (br s, 1H), 7.86 (d, J=12.23 Hz, 1H), 7.52-7.55 (m, 1H), 4.14-4.32 (m, 3H), 3.97 (br s, 3H), 3.75-3.92 (m, 2H), 3.43 (br s, 2H), 2.59-2.62 (m, 3H), 1.45 (d, J=6.85 Hz, 6H).

Step B: 1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-one. A solution of 3-(7-fluoro-4-isopropyl-2-(3-methyl-5-(trifluoromethyl)-1H-pyrazol-4-yl)quinolin-6-yl)-N-methoxy-N,1-dimethyl-1H-1,2,4-triazole-5-carboxamide (70 mg, 0.14 mmol) in THF was cooled to −78° C. A solution of methylmagnesium bromide (3M in diethyl ether, 0.09 mL, 0.28 mmol) was added to the reaction mixture and the reaction mixture was stirred at −78° C. for 15 mins, then warmed to room temperature. The reaction mixture was quenched with sat'd NH₄Cl and extracted (3×) with ethyl acetate. The combined organic layers were washed with brine, dried over Na₂SO₄, filtered, and concentrated under reduced pressure. The title compound (64 mg crude) was obtained as a yellow solid and used in the next step without further purification. MS (ESI): mass calcd. for C₂₂H₂₀F₄N₆O, 460.16; m/z found, 461.1 [M+H]⁺.

Step C: 1-(3-(7-Fluoro-4-isopropyl-2-(3-methyl-5-(trifluoromethyl)-1H-pyrazol-4-ylquinolin-6-yl)-1-methyl-1H-1,2,4-triazol-5 ylethan-1-ol. To a cooled (0° C.) solution of 1-(3-(7-fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-one (64 mg, 0.14 mmol) in MeOH/DCM (4:1) was added sodium borohydride (10.5 mg, 0.28 mmol). The reaction mixture was warmed to room temperature and stirred for 1 hour. The reaction mixture was diluted with water and extracted (3×) with ethyl acetate. The combined organic layers were washed with brine, dried over Na₂SO₄, filtered, and concentrated under reduced pressure. Purification (Phenomenex Gemini-NX, C18, 150×30 mm, 5 um; 30 mL/min; Buffer A: 20 mM NH₄OH/water Buffer B: MeCN; gradient: 20% B for 2 min then linear gradient to 100% B over 12 min) afforded the title compound (20 mg, 32% yield over 2 steps) as a white solid. MS (ESI): mass calcd. for C₂₂H₂₂F₄N₆O, 462.18; m/z found, 463.2 [M+H]⁺. ¹H NMR (400 MHz, DMSO-d₆) δ=8.77 (d, J=7.83 Hz, 1H), 7.80 (d, J=11.74 Hz, 1H), 7.51 (s, 1H), 5.07 (q, J=6.52 Hz, 1H), 4.02 (s, 3H), 3.79 (td, J=6.66, 13.57 Hz, 1H), 2.50-2.52 (m, 3H), 1.57 (d, J=6.36 Hz, 3H), 1.39 (d, J=6.36 Hz, 6H).

### Example 30: (3-(2-(5-Chloro-3-methyl-1H-pyrazol-4-yl)-7-fluoro-4-isopropylauinolin-6-yl)-1-methyl-1H-1,2,4-triazol-5-yl)methanol

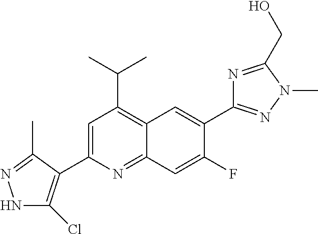 [SMILES: Cc1n[nH]c(Cl)c1-c1cc(C(C)C)c2cc(-c3nc(CO)n(C)n3)c(F)cc2n1]

Title compound was prepared in a manner analogous to Example 28, Steps A-C, except using 3-chloro-5-methyl-4-(4,4,5,5-tetramethyl-1,3,2-dioxaborolan-2-yl)-1H-pyrazole (Intermediate 3 187 mg, 0.78 mmol) instead of 5-methyl-4-(4,4,5,5-tetramethyl-1,3,2-dioxaborolan-2-yl)-3-(trifluoromethyl)-1H-pyrazole (Intermediate 4 in Step A. MS (ESI): mass calcd. for C₂₀H₂₀ClFN₆O, 414.14; m/z found, 415.2 [M+H]⁺. ¹H NMR (400 MHz, DMSO-d₆) δ=13.23 (br s, 1H), 8.76 (d, J=8.31 Hz, 1H), 7.81 (d, J=11.74 Hz, 1H), 7.78 (s, 1H), 5.71 (t, J=5.62 Hz, 1H), 4.72 (d, J=5.87 Hz, 2H), 3.98 (s, 3H), 3.78 (td,J=6.97, 13.45 Hz, 1H), 2.57 (s, 3H), 1.41 (d, J=6.85 Hz, 6H).

### Example 31: 1-(3-(2-(5-Chloro-3-methyl-1H-pyrazol-4-yl)-7-fluoro-4-isopropylauinolin-6-yl)-1-methyl-1H-1,2,4-triazol-5-yl)ethan-1-ol

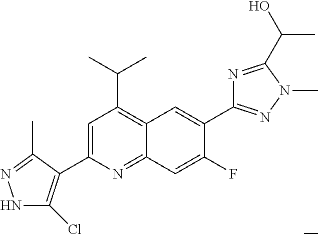 [SMILES: Cc1n[nH]c(Cl)c1-c1cc(C(C)C)c2cc(-c3nc(C(C)O)n(C)n3)c(F)cc2n1]

Title compound was prepared in a manner analogous to Example 29, Steps A-C, except using 3-chloro-5-methyl-4-(4,4,5,5-tetramethyl-1,3,2-dioxaborolan-2-yl)-1H-pyrazole (Intermediate 3, 187 mg, 0.78 mmol) instead of 5-methyl-4-(4,4,5,5-tetramethyl-1,3,2-dioxaborolan-2-yl)-3-(trifluoromethyl)-1H-pyrazole (Intermediate 4) in Step A. MS (ESI): mass calcd. for C₂₁H₂₂ClFN₆O, 428.15; m/z found, 429.1 [M+H]⁺. ¹H NMR (400 MHz, DMSO-d₆) δ=8.73 (d, J=8.31 Hz, 1H), 7.77-7.82 (m, 2H), 5.07 (q, J=6.36 Hz, 1H), 4.01 (s, 3H), 3.77 (td, J=6.85, 13.69 Hz, 1H), 2.56 (s, 3H), 1.56 (d, J=6.36 Hz, 3H), 1.40 (d, J=6.85 Hz, 6H).

### Example 32: (4-(2-(5-Chloro-3-methyl-1H-pyrazol-4-yl)-7-fluoro-4-isopropylauinolin-6-yl)-1-methyl-1H-imidazol-2-yl)methanol

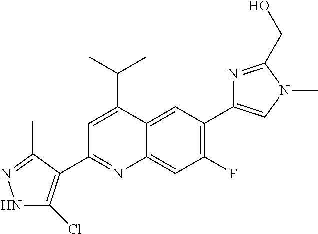 [SMILES: Cc1n[nH]c(Cl)c1-c1cc(C(C)C)c2cc(-c3cn(C)c(CO)n3)c(F)cc2n1]

Step A: Methyl 4-(2-(5-chloro-3-methyl-1H-pyrazol-4-yl)-7-fluoro-4-isopropylquinolin-6-yl)-1-methyl-1H-imidazole-2-carboxylate. A mixture of methyl 4-(2-chloro-7-fluoro-4-isopropylquinolin-6-yl)-1-methyl-1H-imidazole-2-carboxylate (Intermediate 9, 198 mg, 0.547 mmol), 3-chloro-5-methyl-4-(4,4,5,5-tetramethyl-1,3,2-dioxaborolan-2-yl)-1H-pyrazole (Intermediate 3, 239 mg, 0.985 mmol, *1:1 mixture with deborylated pyrazole), XPhos Pd G3 (46 mg, 0.055 mmol), cesium carbonate (535 mg, 1.6 mmol), in dioxane/water (5:1) was purged with argon, and heated to 80° C. for 1 hour. The reaction mixture was cooled to room temperature and subsequently diluted with ethyl acetate and water. The organic layer was extracted (3×) with ethyl acetate and the combined organics were washed with brine, dried over Na₂SO₄, filtered, and concentrated under reduced pressure. The resulting residue was adhered to silica and purified with flash column chromatography (SiO₂, eluting with a gradient of 0-100% ethyl acetate in heptane) to give the title compound (108 mg, 0.24 mmol, 45% yield) as a yellow solid. MS (ESI): mass calcd. for C₂₂H₂₁ClFN₅O₂, 441.14; m/z found, 442.0 [M+H]⁺. (400 MHz, CDCl₃) δ=8.89 (d, J=7.83 Hz, 1H), 7.74-7.78 (m, 2H), 7.65 (d, J=4.40 Hz, 1H), 4.12 (s, 3H), 3.95-4.06 (m, 4H), 2.65 (s, 3H), 1.44-1.47 (m, 6H).

Step B: (4-(2-(5-Chloro-3-methyl-1H-pyrazol-4-yl)-7-fluoro-4-isopropylquinolin-6-yl)-1-methyl-1H-imidazol-2-yl)methanol. A solution of methyl 4-(2-(5-chloro-3-methyl-1H-pyrazol-4-yl)-7-fluoro-4-isopropylquinolin-6-yl)-1-methyl-1H-imidazole-2-carboxylate (105 mg, 0.238 mmol) in THF (5.6 mL) was cooled to 0° C. Sodium borohydride (45 mg, 1.19 mmol) and calcium chloride (16 mg, 0.14 mmol) were added to the reaction mixture and the reaction mixture was heated to 45° C. for 3 hours. The reaction mixture was quenched with sat'd NH₄Cl and diluted with water and ethyl acetate. The reaction mixture was extracted (3×) with ethyl acetate and the combined organics were washed with brine, dried over Na₂SO₄, filtered, and concentrated under reduced pressure. The residue was purified with preparative HPLC. MS (ESI): mass calcd. for C₂₁H₂₁ClFN₅O, 413.14; m/z found, 414.1 [M+H]⁺. ¹H NMR (400 MHz, DMSO-d₆) δ=8.75 (d, J=8.31 Hz, 1H), 7.65-7.76 (m, 3H), 4.60 (s, 2H), 3.72-3.80 (m, 4H), 2.52-2.56 (m, 3H), 1.40 (d, J=6.85 Hz, 6H).

### Example 33: (4-(7-Fluoro-4-isopropyl-2-(3-methyl-5-(trifluoromethyl)-1H-pyrazol-4-yl)quinolin-6-yl)-1-methyl-1H-imidazol-2-yl)methanol

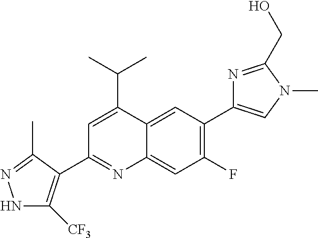 [SMILES: Cc1n[nH]c(C(F)(F)F)c1-c1cc(C(C)C)c2cc(-c3cn(C)c(CO)n3)c(F)cc2n1]

The title compound was prepared in a manner analogous to Example 32, Steps A-B, except using 5-methyl-4-(4,4,5,5-tetramethyl-1,3,2-dioxaborolan-2-yl)-3-(trifluoromethyl)-1H-pyrazole (Intermediate 7) instead of 3-chloro-5-methyl-4-(4,4,5,5-tetramethyl-1,3,2-dioxaborolan-2-yl)-1H-pyrazole (Intermediate 3) in Step A. MS (ESI): mass calcd. for C₂₂H₂₁F₄NSO, 447.17; m/z found, 448.0 [M+H]⁺. ¹H NMR (400 MHz, CDCl₃) δ=8.83 (d, J=8.31 Hz, 1H), 7.75 (d, J=12.72 Hz, 1H), 7.47-7.50 (m, 2H), 4.83 (s, 2H), 3.88-3.97 (m, 1H), 3.79 (s, 3H), 2.57 (s, 3H), 1.42-1.47 (m, 6H).

### Example 34: 4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one

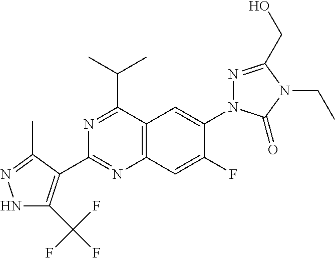 [SMILES: CCn1c(CO)nn(-c2cc3c(C(C)C)nc(-c4c(C)n[nH]c4C(F)(F)F)nc3cc2F)c1=O]

Step A: 5-((Benzyloxy)methyl)-4-ethyl-2-(7-fluoro-4-isopropyl-2-(3-methyl-5-(trifluoromethyl)-1H-pyrazol-4-yl)quinazolin-6-yl)-2,4-dihydro-3H-1,2,4-triazol-3-one. A mixture of 6-bromo-7-fluoro-4-isopropyl-2-(3-methyl-5-(trifluoromethyl)-1H-pyrazol-4-yl)quinazoline (Intermediate 17, 80 mg, 0.19 mmol), (5-((benzyloxy)methyl)-4-ethyl-2,4-dihydro-3H-1,2,4-triazol-3-one) (Intermediate 1, 53.7 mg, 0.23 mmol), copper(I) iodide (18.3 mg, 0.10 mmol), trans-N,N′-dimethylcyclohexane-1,2-diamine (18 μL, 0.12 mmol), and potassium phosphate tribasic (73.3 mg, 0.35 mmol) in 1,4-dioxane (1.5 mL) was heated at 110° C. overnight. The reaction mixture was partitioned between ethyl acetate and H₂O. The organic layer was dried over Na₂SO₄, filtered, and concentrated under reduced pressure. Purification by flash column chromatography (SiO₂ 4G 0-60% heptane/EA) yielded the title compound (50 mg, 46% yield) as a green/yellow oil. MS (ESI): mass calcd. C₂₈H₂₇F₄N₇O₂, 569.22; m/z found 570.2 [M+H]⁺. ¹H NMR (400 MHz, CDCl₃) δ=10.71-11.09 (m, 1H), 8.36 (d, J=7.83 Hz, 1H), 7.73 (d, J=11.25 Hz, 1H), 7.33-7.44 (m, 5H), 4.65 (s, 2H), 4.56 (s, 2H), 3.80-3.96 (m, 3H), 2.71 (s, 3H), 1.36-1.48 (m, 10H).

Step B. 4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one. To a solution of 5-((benzyloxy)methyl)-4-ethyl-2-(7-fluoro-4-isopropyl-2-(3-methyl-5-(trifluoromethyl)-1H-pyrazol-4-yl)quinazolin-6-yl)-2,4-dihydro-3H-1,2,4-triazol-3-one (52 mg, 0.09 mmol) in DCM at −78° C. was added boron trichloride (1M in DCM, 0.46 mL, 0.46 mmol). The reaction mixture was stirred for 1 hour then carefully quenched by the dropwise addition of MeOH followed by water. The organics were extracted with DCM, washed with brine, dried over Na₂SO₄, filtered, and concentrated under reduced pressure. Purification by reverse phase chromatography (Phenomenex Gemini-NX, C18, 150×30 mm, 5 um; 30 mL/min; Buffer A: 20 mM NH₄OH/water Buffer B: MeCN; gradient: 20% B for 2 min then linear gradient to 100% B over 10 min) yielded the title compound (15 mg, 0.031 mmol, 34% yield) as a white solid. MS (ESI): mass calcd. C₂₁H₂₁F₄N₇O₂, 479.17; m/z found 480.1 [M+H]⁺. (400 MHz, CDCl₃) δ=10.01-10.16 (m, 1H), 8.39 (d, J=7.83 Hz, 1H), 7.76 (d, J=11.25 Hz, 1H), 4.71 (d, J=6.36 Hz, 2H), 3.91-3.99 (m, 2H), 3.82-3.90 (m, 1H), 2.79 (s, 3H), 2.09 (t, J=6.11 Hz, 1H), 1.43-1.49 (m, 9H).

### Example 35: 1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one

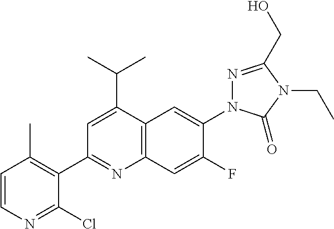 [SMILES: CCn1c(CO)nn(-c2cc3c(C(C)C)cc(-c4c(C)ccnc4Cl)nc3cc2F)c1=O]

Step A. 3-((Benzyloxy)methyl)-1-(2-(2-chloro-4-methylpyridin-3-yl)-7-fluoro-4-isopropylquinolin-6-yl)-4-ethyl-1H-1,2,4-triazol-5 (4H)-one. To a mixture of 3-((benzyloxy)methyl)-1-(2-chloro-7-fluoro-4-isopropylquinolin-6-yl)-4-ethyl-1H-1,2,4-triazol-5 (4H)-one (Intermediate 2, 100 mg, 219.8 μmol), 2-chloro-4-methyl-3-(4,4,5,5-tetramethyl-1,3,2-dioxaborolan-2-yl)pyridine (Intermediate 18, 52.9 mg, 208.8 μmol) and Pd(dppf)Cl₂ (35.9 mg, 43.96 μmol) in 1,4-dioxane (3 mL) was added 2 M aq. Na₂CO₃ (0.22 mL, 0.44 mmol, 2 eq.). The reaction mixture was stirred at 100° C. for 1 hour under N₂. The reaction mixture was diluted with ethyl acetate (30 mL) and filtered. The filtrate was concentrated under reduced pressure. The crude was purified by column chromatography (SiO₂, gradient elution: 0-50% ethyl acetate in petroleum ether) to give the title compound (91.7 mg, 168 μmol, 76% yield) as light brown gum. MS (ESI): mass calcd. for C₃₀H₂₉ClFN₅O₂, 545.2; m/z found, 546.2 [M+H]⁺. ¹H NMR (400 MHz, CDCl₃) S=8.40 (d, J=8.0 Hz, 1H), 8.35 (d, J=5.0 Hz, 1H), 7.93 (d, J=11.3 Hz, 1H), 7.45-7.32 (m, 6H), 7.24 (d, J=5.0 Hz, 1H), 4.66 (s, 2H), 4.57 (s, 2H), 3.92 (q, J=7.1 Hz, 2H), 3.76 (td, J=6.6, 13.6 Hz, 1H), 2.20 (s, 3H), 1.44 (d, J=6.8 Hz, 6H), 1.43-1.38 (m, 3H); ¹⁹F NMR (376 MHz, CDCl₃) δ=−117.85 (1F) ppm.

Step B. 1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one. BCl₃ (1 M solution in toluene, 1.01 mL, 1.01 mmol, 5.0 eq.) was added to a stirred solution of 3-((benzyloxy)methyl)-1-(2-(2-chloro-4-methylpyridin-3-yl)-7-fluoro-4-isopropylquinolin-6-yl)-4-ethyl-1H-1,2,4-triazol-5 (4H)-one (110 mg, 201.5 μmol, 1.0 eq.) in DCM (5 mL) at −78° C. The reaction mixture was stirred at −78° C. for 1 hour. The reaction mixture was quenched with MeOH (2 mL) at −78° C. and stirred at −78° C. for 0.5 hour. A sat. aq. NaHCO₃ solution (20 mL) was added to the reaction mixture, and the reaction mixture was extracted with DCM (20 mL×2). The combined organic phase was dried with anhydrous Na₂SO₄, filtered, and concentrated under reduced pressure. The crude product was purified by preparative reversed phase HPLC (Stationary phase: Phenomenex Gemini NX-C18, 3 μm, 75×30 mm; Mobile phase: water (0.05% NH₃H₂O+10 mM NH₄HCO₃) (A) —MeCN (B), gradient elution: 32-62% B in A over 7 min, flow rate: 25 mL/min) to give the title compound (60.0 mg, 131.9 μmol, 75.3% yield) as white powder. MS (ESI): mass calcd. For C₂₃H₂₃ClFN₅O₂, 455.9; m/z found, 456.1 [M+H]⁺. ¹H NMR (400 MHz, CDCl₃) δ=8.40 (d, J=8.0 Hz, 1H), 8.35 (d, J=5.0 Hz, 1H), 7.93 (d, J=11.3 Hz, 1H), 7.37 (s, 1H), 7.24 (d, J=5.0 Hz, 1H), 4.71 (d, J=6.0 Hz, 2H), 3.96 (q, J=7.1 Hz, 2H), 3.76 (spt, J=6.8 Hz, 1H), 2.54 (br t, J=5.9 Hz, 1H), 2.19 (s, 3H), 1.49-1.45 (m, 3H), 1.44 (d, J=6.8 Hz, 6H); ¹⁹F NMR (376 MHz, CDCl₃) δ=−117.91 (1F) ppm.

### Example 36: (S*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one

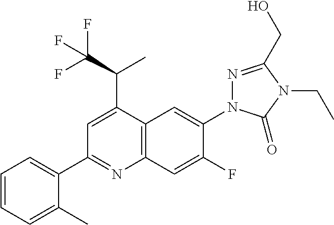 [SMILES: CCn1c(CO)nn(-c2cc3c([C@H](C)C(F)(F)F)cc(-c4ccccc4C)nc3cc2F)c1=O]

Step A. 3-((Benzyloxy)methyl)-4-ethyl-1-(7-fluoro-2-hydroxy-4-(1,1,1-trifluoropropan-2-yl)quinolin-6-yl)-1H-1,2,4-triazol-5 (4H)-one. To a solution of 6-bromo-7-fluoro-4-(1,1,1-trifluoropropan-2-yl)quinolin-2-ol (Intermediate 19, 140 mg, 344.78 μmol, 1 eq.), 3-((benzyloxy)methyl)-4-ethyl-1H-1,2,4-triazol-5 (4H)-one (Intermediate 1, 120.64 mg, 517.17 μmol, 1.5 eq.), Cs₂CO₃ (359.5 mg, 1.10 mmol, 3.2 eq.), (1R,2R)—N₁,N₂-dimethylcyclohexane-1,2-diamine (137.3 mg, 965.39 μmol, 2.8 eq.) in dioxane (4 mL) was added CuI (131.3 mg, 689.6 μmol, 2 eq.). The reaction mixture was degassed and purged with N₂ for 3 times and stirred at 110° C. for 16 hours under N₂ atmosphere. The reaction mixture was filtered, and the filtrate was concentrated under reduced pressure to give the residue. The residue was purified by flash column chromatography (SiO₂, gradient elution: 0-10% MeOH in DCM) to give the title compound as light brown gum. (165 mg, 305.2 μmol, 88.52% yield). MS (ESI): mass calcd. For C₂₄H₂₂F₄N₄O₃, 490.16; m/z found, 491.1 [M+H]⁺. ¹H NMR (400 MHz, CDCl₃) δ=12.07 (br s, 1H), 9.34 (br s, 1H), 7.90 (d, J=7.3 Hz, 1H), 7.39-7.36 (m, 5H), 6.85 (s, 1H), 4.63 (s, 2H), 4.53 (s, 2H), 4.00 (td, J=7.7, 15.6 Hz, 1H), 3.88 (q, J=7.2 Hz, 2H), 1.59 (br s, 3H), 1.39 (t, J=7.2 Hz, 3H); ¹⁹F NMR (376 MHz, CDCl₃) δ=−69.75 (3F), −114.30 (1F) ppm.

Step B. 3-((Benzyloxy)methyl)-1-(2-chloro-7-fluoro-4-(1,1,1-trifluoropropan-2-yl)quinolin-6-yl)-4-ethyl-1H-1,2,4-triazol-5 (4H)-one. POCl₃ (998.30 mg, 6.51 mmol, 22 eq.) was added slowly into a solution of 3-((benzyloxy)methyl)-4-ethyl-1-(7-fluoro-2-hydroxy-4-(1,1,1-trifluoropropan-2-yl)quinolin-6-yl)-1H-1,2,4-triazol-5 (4H)-one (160 mg, 295.94 μmol, 90.7% purity, 1 eq.) in toluene (1.5 mL) at r.t. The reaction mixture was stirred at 95° C. for 1 hour. The reaction mixture was concentrated under reduced pressure. The resulting residue was diluted with ethyl acetate (10 mL), then alkalized with sat. aq. NaHCO₃ (3 mL). The reaction mixture was extracted with ethyl acetate (30 mL×2). The combined organic layers were dried over Na₂SO₄, filtered, then concentrated under reduced pressure to give the crude product. The crude product was purified by flash column chromatography (SiO₂, gradient elution: 0-10% MeOH in DCM) to give the title compound as light brown gum. (115 mg, 205.94 μmol, 69% yield). MS (ESI): mass calcd. for C₂₄H₂₁ClF₄N₄O₂, 508.13; m/z found, 509.2[M+H]⁺. ¹H NMR (400 MHz, CDCl₃) δ=8.25 (d, J=7.5 Hz, 1H), 7.88 (d, J=11.0 Hz, 1H), 7.49 (s, 1H), 7.42-7.34 (m, 5H), 4.65 (s, 2H), 4.56 (s, 2H), 4.27 (td, J=7.6, 15.2 Hz, 1H), 3.90 (q, J=7.2 Hz, 2H), 1.65 (d, J=7.3 Hz, 3H), 1.40 (t, J=7.2 Hz, 3H); ¹⁹F NMR (376 MHz, CDCl₃) δ=−69.77 (3F), −114.57 (1F) ppm.

Step C. 3-((Benzyloxy)methyl)-4-ethyl-1-(7-fluoro-2-(o-tolyl)-4-(1,1,1-trifluoropropan-2-yl)quinolin-6-yl)-1H-1,2,4-triazol-5 (4H)-one. A mixture of 3-((benzyloxy)methyl)-1-(2-chloro-7-fluoro-4-(1,1,1-trifluoropropan-2-yl)quinolin-6-yl)-4-ethyl-1H-1,2,4-triazol-5 (4H)-one (110 mg, 196.98 μmol, 91.13% purity, 1 eq.), o-tolylboronic acid (80.34 mg, 590.9 μmol, 3 eq.), Pd(118) (12.84 mg, 19.7 μmol, 0.1 eq.) and K₂CO₃ (81.7 mg, 590.9 μmol, 3 eq.) in dioxane/H₂O (v/v, 5/1, 2.5 mL) was bubbled with N₂ for 5 mins. The reaction mixture was stirred at 90° C. for 16 hours. The reaction mixture was concentrated under reduced pressure to give the residue. The resulting residue was purified by flash column chromatography (SiO₂, gradient elution: 0-50% ethyl acetate in petroleum ether) to give the title compound (100 mg, 177.12 μmol, 89.9% yield). MS (ESI): mass calcd. for C₃₁H₂₈F₄N₄O₂, 564.21; m/z found, 565.1[M+H]⁺. ¹H NMR (400 MHz, CDCl₃) S=8.28 (d, J=7.5 Hz, 1H), 8.00 (d, J=11.5 Hz, 1H), 7.66 (s, 1H), 7.58-7.52 (m, 1H), 7.43-7.33 (m, 8H), 4.66 (s, 2H), 4.57 (s, 2H), 4.35 (td, J=7.8, 15.4 Hz, 1H), 3.92 (q, J=7.3 Hz, 2H), 2.43 (s, 3H), 1.67 (d, J=7.0 Hz, 3H), 1.41 (t, J=7.2 Hz, 3H); ¹⁹F NMR (376 MHz, CDCl₃) δ=−69.83 (3F), −117.28 (1F) ppm.

Step D. 4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one. BCl₃ (0.89 mL, 885.6 μmol, 1M in toluene) was added to 3-((benzyloxy)methyl)-4-ethyl-1-(7-fluoro-2-(o-tolyl)-4-(1,1,1-trifluoropropan-2-yl)quinolin-6-yl)-1H-1,2,4-triazol-5 (4H)-one (100 mg, 177.12 μmol) in DCM (10 mL) at −78° C. under N₂. The reaction mixture was stirred at −78° C. for 1 hour. The reaction mixture was quenched with MeOH (2 mL) at −78° C., and stirred at −78° C. for 0.5 hour. The reaction mixture was diluted with DCM (30 mL) and washed with sat. aq. NaHCO₃ (9 mL). The organic layer was dried with Na₂SO₄, filtered, and concentrated under reduced pressure to give the residue. The residue was purified by column chromatography (SiO₂, gradient elution: 0-50% ethyl acetate in petroleum ether) to give the title compound (83 mg, 174.9 μmol, 98.8% yield). MS (ESI): mass calcd. for C₂₄H₂₂F₄N₄O₂, 474.17; m/z found, 475.1[M+H]⁺. ¹H NMR (400 MHz, CDCl₃) δ=8.29 (d, J=7.8 Hz, 1H), 8.01 (d, J=11.3 Hz, 1H), 7.66 (s, 1H), 7.57-7.52 (m, 1H), 7.54 (d, J=7.3 Hz, 1H), 7.42-7.34 (m, 3H), 4.73 (d, J=6.0 Hz, 2H), 4.35 (td, J=7.5, 15.4 Hz, 1H), 3.96 (q, J=7.0 Hz, 2H), 2.43 (s, 3H), 2.23-2.15 (m, 1H), 1.67 (d, J=7.0 Hz, 3H), 1.47 (t, J=7.3 Hz, 3H); ¹⁹F NMR (376 MHz, CDCl₃) δ=−69.84 (3F), −117.37 (1F) ppm.

Step E. (S*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one. 4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one (83 mg, 174.94 μmol) was separated by chiral SFC: (Column: Cellulose 2 150×4.6 mM I.D., 5 um; mobile phase: CO₂(A) —MeOH (0.05% DEA) (B); Gradient: from 5% to 40% of B in 5 min, then from 40% to 5% of B for 0.5 min; hold 5% of B for 1.5 min; column temp.:35° C.; ABPR: 1500 psi) to give two title compounds.

The first eluting compound (24.4 mg, 50.51 μmol, 28.87% yield, 98.22% purity) as white solid. MS (ESI): mass calcd. for C₂₄H₂₂F₄N₄O₂, 474.17; m/z found, 475.2 [M+H]⁺. ¹H NMR (400 MHz, CDCl₃) δ=8.29 (d, J=7.5 Hz, 1H), 8.00 (d, J=11.3 Hz, 1H), 7.66 (s, 1H), 7.57-7.52 (m, 1H), 7.41-7.33 (m, 3H), 4.73 (s, 2H), 4.35 (td, J=8.0, 15.7 Hz, 1H), 3.96 (q, J=7.3 Hz, 2H), 2.43 (s, 3H), 1.67 (d, J=7.0 Hz, 3H), 1.47 (t, J=7.3 Hz, 3H); ¹⁹F NMR (376 MHz, CDCl₃) δ=−69.85 (3F), −117.42 (1F) ppm.

### Example 37: (R*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one

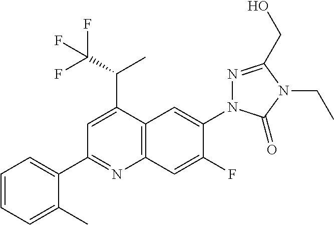 [SMILES: CCn1c(CO)nn(-c2cc3c([C@@H](C)C(F)(F)F)cc(-c4ccccc4C)nc3cc2F)c1=O]

The second eluting compound from Example 37 (23.0 mg, 47.77 μmol, 27.30% yield, 98.54% purity) as white solid. MS (ESI): mass calcd. for C₂₄H₂₂F₄N₄O₂, 474.17; m/z found, 475.1 [M+H]⁺. ¹H NMR (400 MHz, CDCl₃) δ=8.29 (d, J=7.5 Hz, 1H), 8.00 (d, J=11.3 Hz, 1H), 7.66 (s, 1H), 7.57-7.52 (m, 1H), 7.43-7.32 (m, 3H), 4.73 (s, 2H), 4.35 (td, J=7.7, 15.7 Hz, 1H), 3.96 (q, J=7.1 Hz, 2H), 2.43 (s, 3H), 1.67 (d, J=7.0 Hz, 3H), 1.47 (t, J=7.3 Hz, 3H); ¹⁹F NMR (376 MHz, CDCl₃) δ=−69.85 (3F), −117.42 (1F) ppm.

### Example 38: 4-Ethyl-1-(7-fluoro-4-isopropyl-2-(o-methyl-d₃-phenyl)quinolin-6-yl)-3-(hydroxymethyl)-1H-1,2,4-triazol-5 (4H)-one

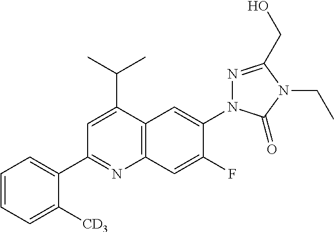 [SMILES: [2H]C([2H])([2H])c1ccccc1-c1cc(C(C)C)c2cc(-n3nc(CO)n(CC)c3=O)c(F)cc2n1]

The title compound was prepared in a manner analogous to Example 1, Steps A-B, except using 4,4,5,5-tetramethyl-2-(o-methyl-d₃-phenyl)-1,3,2-dioxaborolane (Intermediate 14, 194.4 mg, 880 μmol) instead of (2-methoxyphenyl)boronic acid in Step A. MS (ESI): mass calcd. For C₂₄H₂₂D₃FN₄O₂, 423.2; m/z found, 424.3 [M+H]⁺. ¹H NMR (400 MHz, CDCl₃) δ=8.35 (d, J=8.0 Hz, 1H), 7.95 (br d, J=10.8 Hz, 1H), 7.56-7.50 (m, 1H), 7.46 (s, 1H), 7.42-7.31 (m, 3H), 4.72 (d, J=6.2 Hz, 2H), 3.96 (q, J=7.2 Hz, 2H), 3.82-3.67 (m, 1H), 2.23 (br t, J=6.2 Hz, 1H), 1.48-1.42 (m, 9H); ¹F NMR (376 MHz, CDCl₃) δ=−119.03 (1F) ppm.

### Example 39: 4-Ethyl-1-(7-fluoro-4-(prop-1-en-2-Yl)-2-(o-tolyl)quinolin-6-yl)-3-(hydroxymethyl)-1H-1,2,4-triazol-5 (4H)-one

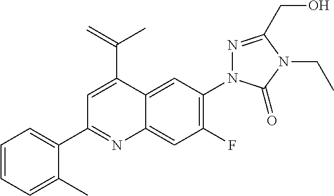 [SMILES: C=C(C)c1cc(-c2ccccc2C)nc2cc(F)c(-n3nc(CO)n(CC)c3=O)cc12]

Step A. 3-((Benzyloxy)methyl)-4-ethyl-1-(7-fluoro-2,4-dihydroxyquinolin-6-yl)-1H-1,2,4-triazol-5 (4H)-one. To a mixture of 6-bromo-7-fluoroquinoline-2,4-diol (Intermediate 20, 1 g, 3.41 mmol) and 3-((benzyloxy)methyl)-4-ethyl-1H-1,2,4-triazol-5 (4H)-one (Intermediate 1, 2.39 g, 10.23 mmol) in dioxane (15 mL) was added Cs₂CO₃ (2.00 g, 6.14 mmol), KI (566.11 mg, 3.41 mmol) and (1R,2R)—N₁,N₂-dimethylcyclohexane-1,2-diamine (388.07 mg, 2.73 mmol), followed by CuI (649.5 mg, 3.41 mmol) under N₂. The reaction mixture was heated to 110° C. and stirred for 16 hours. The reaction mixture was poured into water (50 mL) and extracted with ethyl acetate (50 mL×2). The organic phase was concentrated in vacuum to give the title compound (4 g, crude) as a black brown solid. MS (ESI): mass calcd For C₂₁H₁₁FN₄O₄, 410.1; m/z found, 411.4 [M+H]⁺.

Step B 3-((Benzyloxy)methyl)-1-(2,4-dichloro-7-fluoroquinolin-6-yl)-4-ethyl-1H-1,2,4-triazol-5 (4H)-one. A mixture of 3-((benzyloxy)methyl)-4-ethyl-1-(7-fluoro-2,4-dihydroxyquinolin-6-yl)-1H-1,2,4-triazol-5 (4H)-one (4 g, 9.75 mmol) in POCl₃ (51.15 g, 333.6 mmol, 31 mL) was stirred at 110° C. for 1 hr. The reaction mixture was concentrated under reduced pressure. The resulting residue was poured into sat.NaHCO₃ (100 mL) and extracted with ethyl acetate (50 mL×2). The combined organic phase was washed with brine (75 mL×2), dried with anhydrous Na₂SO₄, filtered, and concentrated under reduced pressure. The residue was purified by flash column chromatography (SiO₂, Petroleum ether/Ethyl acetate=30/1 to 10/1 to 3/1) to give the title compound (540 mg, 1.05 mmol, 11% yield) as a black brown oil. MS (ESI): mass calcd For C₂₁H₁₇Cl₂FN₄O₂, 446.1; m/z found, 447.3 [M+H]⁺. ¹H NMR (400 MHz, CDCl₃) δ=8.45 (d, J=7.7 Hz, 1H), 7.85 (d, J=10.9 Hz, 1H), 7.54 (s, 1H), 7.41-7.31 (m, 5H), 4.64 (s, 2H), 4.55 (s, 2H), 3.89 (q, J=7.2 Hz, 2H), 1.39 (t, J=7.2 Hz, 3H).

Step C. 3-((Benzyloxy)methyl)-1-(4-chloro-7-fluoro-2-(o-tolyl)quinolin-6-yl)-4-ethyl-1H-1,2,4-triazol-5 (4H)-one. To a mixture of 3-((benzyloxy)methyl)-1-(2,4-dichloro-7-fluoroquinolin-6-yl)-4-ethyl-1H-1,2,4-triazol-5 (4H)-one (195 mg, 435.96 μmol), o-tolylboronic acid (118.54 mg, 871.9 μmol), Na₂CO₃ (115.5 mg, 1.09 mmol) in dioxane (3 mL) and H₂O (0.3 mL) was added Pd(dppf)Cl₂ (31.9 mg, 43.6 μmol) under N₂. The reaction mixture was stirred at 80° C. for 15 hrs under N₂ atmosphere. The reaction mixture was poured into water (10 mL) and extracted with ethyl acetate (10 mL×2). The combined organic phase was washed with brine (15 mL×2), dried with anhydrous Na₂SO₄, filtered, and concentrated under reduced pressure. The residue was purified by prep-TLC (SiO₂, Petroleum ether: Ethyl acetate=3:1) to give the title compound (215 mg, 403.6 μmol, 93% yield) as a white solid. MS (ESI): mass calcd For C₂₁H₁₇Cl₂FN₄O₂, 446.1; m/z found, 447.3 [M+H]⁺.

Step D. 3-((Benzyloxy)methyl)-4-ethyl-1-(7-fluoro-4-(prop-1-en-2-yl)-2-(o-tolyl)quinolin-6-yl)-1H-1,2,4-triazol-5 (4H)-one. To a mixture of 3-((benzyloxy)methyl)-1-(4-chloro-7-fluoro-2-(o-tolyl)quinolin-6-yl)-4-ethyl-1H-1,2,4-triazol-5 (4H)-one (65 mg, 129.2 μmol), 4,4,5,5-tetramethyl-2-(prop-1-en-2-yl)-1,3,2-dioxaborolane (108.6 mg, 646.2 μmol), Na₂CO₃ (95.9 mg, 904.6 μmol) in dioxane (1 mL) and H₂O (0.2 mL) was added Pd(dppf)Cl₂ (18.9 mg, 25.9 μmol) under N₂. The reaction mixture was stirred at 80° C. for 3 hrs under N₂ atmosphere. The reaction mixture was poured into water (10 mL) and extracted with ethyl acetate (10 mL×2). The combined organic phase was washed with brine (15 mL×2), dried with anhydrous Na₂SO₄, filtered, and concentrated under reduced pressure. The residue was purified by prep-TLC (SiO₂, Petroleum ether/Ethyl acetate=3/1) to give the title compound (36 mg, 70.7 μmol, 55% yield) as a colorless oil. MS (ESI): mass calcd For C₃₁H₂₉FN₄O₂, 508.2; m/z found, 509.4 [M+H]⁺.

Step E. 4-Ethyl-1-(7-fluoro-4-(prop-1-en-2-yl)-2-(o-tolyl)quinolin-6-yl)-3-(hydroxymethyl)-1H-1,2,4-triazol-5 (4H)-one. To a solution of 3-((benzyloxy)methyl)-4-ethyl-1-(7-fluoro-4-(prop-1-en-2-yl)-2-(o-tolyl)quinolin-6-yl)-1H-1,2,4-triazol-5 (4H)-one (35 mg, 68.8 μmol) in DCM (1 mL) was added BCl₃ (1 M, 206.5 μL). The reaction mixture was stirred at −78° C. for 1 hr. The reaction mixture was poured into water (5 mL) and extracted with ethyl acetate (5 mL×3). The combined organic phase was washed with brine (8 mL×2), dried with anhydrous Na₂SO₄, filtered, and concentrated under reduced pressure. The residue was purified by prep-HPLC (METHOD A) to give the title compound (19.58 mg, 45.4 μmol, 66% yield) as a white solid. MS (ESI): mass calcd For C₂₄H₂₃FN₄O₂, 418.2; m/z found, 419.2 [M+H]⁺. ¹H NMR (400 MHz, CDCl₃) δ=8.31 (d, J=8.3 Hz, 1H), 7.94 (d, J=11.5 Hz, 1H), 7.55-7.49 (m, 1H), 7.40-7.32 (m, 4H), 5.54 (t, J=1.5 Hz, 1H), 5.24 (s, 1H), 4.68 (s, 2H), 3.96-3.91 (m, 2H), 2.48 (s, 1H), 2.44 (s, 3H), 2.25 (s, 3H), 1.44 (t, J=7.2 Hz, 3H).

### Biological Data

DHODH inhibitory activities of the compounds of Examples 1-39 were assessed using the following assays. The half maximal effective concentration values (IC₅₀) are summarized in Table 2.

### BIOLOGICAL ASSAYS

### In Vitro Assay: DHODH Enzymatic Assay

To detect DHODH enzyme activities, dichloroindophenol (DCIP) is added as the final electron acceptor in the assay. DCIP can accept electrons from the reduced coenzyme Q generated in the assay, or from dihydroorotate (DHO) via FMN by binding presumably to the ubiquinone pocket. DCIP solutions are blue, with an intense absorbance around 600 nm, but becomes colorless upon reduction (J. Biol. Chem. (1986) 261, 11386). The assay buffer contained 50 nM HEPES, pH 7.5, 150 mM NaCl, 0.5 mM EDTA, and 0.1% Triton X-100 in MilliQ water. Substrate consisting of 20 mM DHO, 5 mM CoQ₆, and 1 mM DCIP in assay buffer, initiates the reaction. The assay is run in end-point mode by quenching the reaction with the potent DHODH inhibitor brequinar. Absorbance measurements were obtained using the BMG Phera Star plate-reading spectrophotomer. Purified human DHODH was purchased from Proteros (cat. No. PR-0044). Chemicals were purchased from Sigma-Aldrich, Teknova, and Avanti Polar Lipids. Liquid handling was performed using Labcyte Echo and Formulatrix Tempest.

### In Vitro Assay: MOLM-13 Cellular Assay

MOLM-13 cells were obtained from DSMZ and were maintained in RPMI 1640+Glutamax+25 mM HEPES (Invitrogen, catalog number 72400) supplemented with 10% heat inactivated fetal bovine serum (FBS; Invitrogen, catalog number 16140). The day prior to assay set-up, cells were pelleted, resuspended in fresh media, counted, and cells were plated at 0.4×10⁶ cell/mL in a T150 flask. On the day of the assay, cells were pelleted, resuspend in fresh media, counted, and seeded at 5,000 cells/well in white opaque 96-well tissue culture treated microplates (Perkin Elmer, catalog number 6005680). Cells were exposed to different concentrations of test compounds at 37° C., 5% CO₂ for 72 hours immediately after seeding. Cell viability was acquired on a Perkin Elmer Envision 2104 multilabel reader using the CellTiter-Glo assay (Promega) according to the manufacturer's instructions.

TABLE 2

| | DHODH Enzymatic | MOLM-13 Cellular |
|---|---|---|
| | Assay IC₅₀ | Assay IC₅₀ |
| Example # | (nM) | (nM) |
| 1 | 3.3 | 2.6 |
| 2 | 0.49 | 1.7 |
| 3 | 0.58 | 0.7 |
| 4 | 1.7 | 8.4 |
| 5 | 332 | >100 |
| 6 | 1083 | >100 |
| 7 | 1.5 | 4 |
| 8 | 4.8 | 9.1 |
| 9 | 0.6 | 0.5 |
| 10 | 1.9 | 2.2 |
| 11 | 32 | 30 |
| 12 | 2.3 | 5 |
| 13 | 270 | >100 |
| 14 | 120 | 100 |
| 15 | 13 | 20 |
| 16 | 9.4 | >30 |
| 17 | 230 | 40 |
| 18 | 2280 | >100 |
| 19 | 12 | >100 |
| 20 | 730 | >100 |
| 21 | 4.6 | 5 |
| 22 | 3.9 | >100 |
| 23 | 14 | 9 |
| 24 | 33 | >100 |
| 25 | 11 | 6 |
| 26 | 13 | 7 |
| 27 | 0.7 | 6 |
| 28 | 3 | 2 |
| 29 | 1 | 0.36 |
| 30 | 1.2 | 2 |
| 31 | 1.6 | 0.83 |
| 32 | 1.2 | 3 |
| 33 | 2.0 | 1 |
| 34 | 5.2 | 4 |
| 35 | 4.7 | 4 |
| 36 | 0.32 | 0.4 |
| 37 | 5.3 | 1 |
| 38 | 1.7 | NT |
| 39 | 0.24 | 0.2 |

NT means not tested

### ENUMERATED EMBODIMENTS

Exemplary numbered embodiments of the invention are shown below.
1. A compound having the structure of Formula (I):

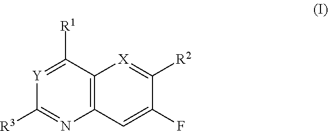

wherein
X is CH or, optionally, N;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆-haloalkyl substituted with OH, or OCH₃; C₁₋₆cycloalkyl; C₁₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of:

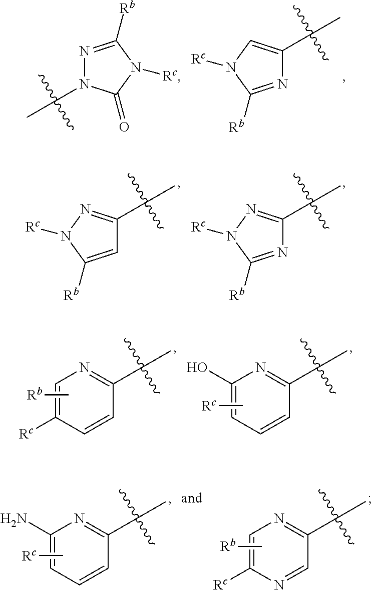

where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₁₋₆cycloalkyl, and tetrahydro-2H-pyranyl; and
R³ is selected from the group consisting of:
   (a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl;

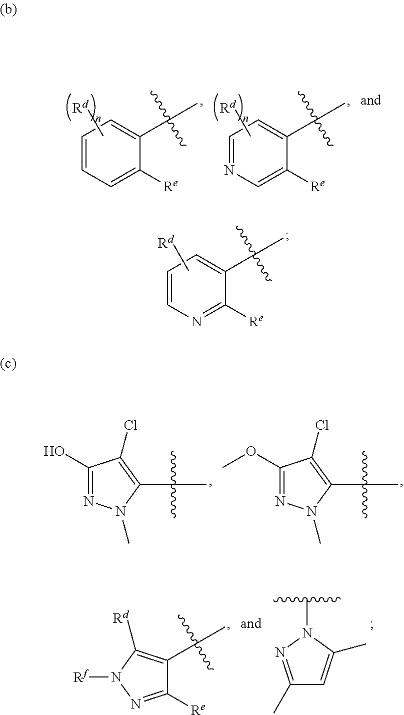

where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆-haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₃₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, solvate, stereoisomer, tautomer, isotopic variant, or N-oxide thereof.
2. The compound according to embodiment 1, wherein X is CH; or a pharmaceutically acceptable salt, solvate, stereoisomer, tautomer, isotopic variant, or N-oxide thereof.
3. The compound according to embodiment 1 or 2, wherein Y is CH; or a pharmaceutically acceptable salt, solvate, stereoisomer, tautomer, isotopic variant, or N-oxide thereof.
4. The compound according to embodiment 1 or 2 wherein Y is N; or a pharmaceutically acceptable salt, solvate, stereoisomer, tautomer, isotopic variant, or N-oxide thereof.
5. The compound according to any of embodiments 1-4, wherein R¹ is C₁₋₆alkyl; C₁₋₄alkyl substituted with OH, or OCH₃; C₂₋₆alkenyl; C₁₋₄haloalkyl; C₁₋₄haloalkyl substituted with OH, or OCH₃; C₃₋₆cycloalkyl; C₃₋₆cycloalkyl independently substituted with one, two, three or four members each independently selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.
6. The compound according to any of embodiments 1-4, wherein R¹ is CH(CH₃)₂; or a pharmaceutically acceptable salt, solvate, stereoisomer, tautomer, isotopic variant, or N-oxide thereof.
7. The compound according to any of embodiments 1-4, wherein R¹ is CH(CH₃XCF₃); or a pharmaceutically acceptable salt, solvate, stereoisomer, tautomer, isotopic variant, or N-oxide thereof.
8. The compound according to any of embodiments 1-4, wherein R¹ is

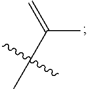 [SMILES: *C(=C)C]

or a pharmaceutically acceptable salt, solvate, stereoisomer, tautomer, isotopic variant, or N-oxide thereof.
9. The compound according to any of embodiments 1-4, wherein R¹ is cyclopropyl, cyclobutyl, cyclopentyl, or cyclohexyl; cyclopropyl, cyclobutyl, cyclopentyl, or cyclohexyl each independently substituted with one, two, three or four members selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.
10. The compound according to any of embodiments 1-9, wherein R² is

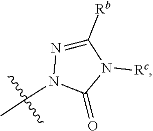

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
11. The compound according to any of embodiments 1-9, wherein R² is

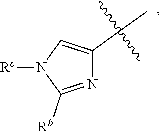

where
R^{b} is C₁₋₄alkyl substituted with OH; and R^{c} is C₁₋₄alkyl; or a pharmaceutically acceptable salt, solvate, stereoisomer, tautomer, isotopic variant, or N-oxide thereof.
12. The compound according to any of embodiments 1-9, wherein R² is

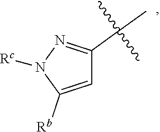

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
13. The compound according to any of embodiments 1-9, wherein R² is

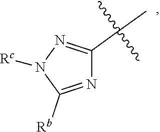

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
14. The compound according to any of embodiments 1-9, wherein R² is

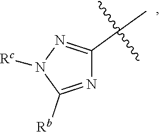

where
R^{b} is C₁₋₄alkyl substituted with OH; and R^{c} is C₁₋₄alkyl; or a pharmaceutically acceptable salt, solvate, stereoisomer, tautomer, isotopic variant, or N-oxide thereof.
15. The compound according to any of embodiments 1-9, wherein R² is

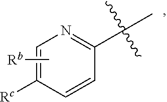

where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
16. The compound according to any of embodiments 1-9, wherein R² is

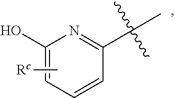

where
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
17. The compound according to any of embodiments 1-9, wherein R² is

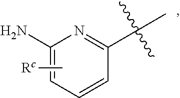

where
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
18. The compound according to any of embodiments 1-9, wherein R² is

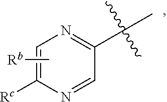

R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.
19. The compound according to any of embodiments 1-9, wherein R² is

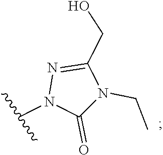 [SMILES: *n1nc(CO)n(CC)c1=O]

or a pharmaceutically acceptable salt, solvate, stereoisomer, tautomer, isotopic variant, or N-oxide thereof.
20. The compound according to any of embodiments 1-19, wherein R³ is O—(C₁₋₄alkyl), N(C₁₋₄alkyl)₂, piperidinyl, piperidinyl substituted with CH₃, O—C₃₋₆cycloalkyl, or N—C₃₋₆cycloalkyl; or a pharmaceutically acceptable salt, solvate, stereoisomer, tautomer, isotopic variant, or N-oxide thereof.
21. The compound according to any of embodiments 1-19, wherein R³ is

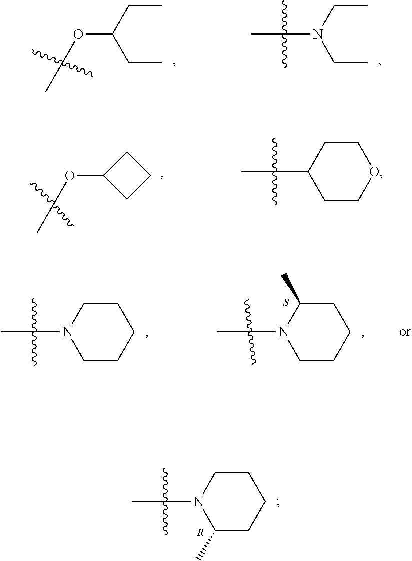

or a pharmaceutically acceptable salt, solvate, stereoisomer, tautomer, isotopic variant, or N-oxide thereof.
22. The compound according to any of embodiments 1-19, wherein R³ is

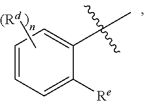

where
R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; and OC₁₋₄alkyl;
R^{e} is halo; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃; and
n is 1 or 2;
or a pharmaceutically acceptable salt, solvate, stereoisomer, tautomer, isotopic variant, or N-oxide thereof.
23. The compound according to any of embodiments 1-19, wherein R³ is

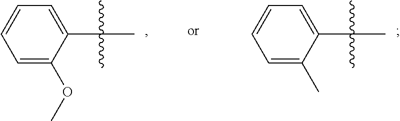

or a pharmaceutically acceptable salt, solvate, stereoisomer, tautomer, isotopic variant, or N-oxide thereof.
24. The compound according to any of embodiments 1-19, wherein R³ is

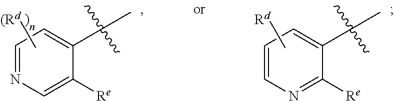

wherein
R^{d} is independently selected from the group consisting of: CH₃, OCH₃ and OH; R is halo, CH₃, or OCH₃; and n is 1 or 2;
or a pharmaceutically acceptable salt, solvate, stereoisomer, tautomer, isotopic variant, or N-oxide thereof.
25. The compound according to any of embodiments 1-19, wherein R³ is

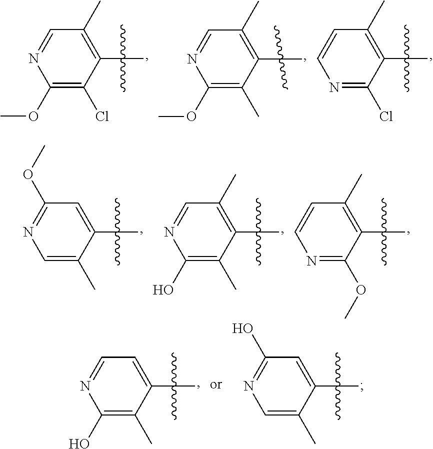

or a pharmaceutically acceptable salt, solvate, stereoisomer, tautomer, isotopic variant, or N-oxide thereof.
26. The compound according to any of embodiments 1-19, wherein R³ is selected from the group consisting of:

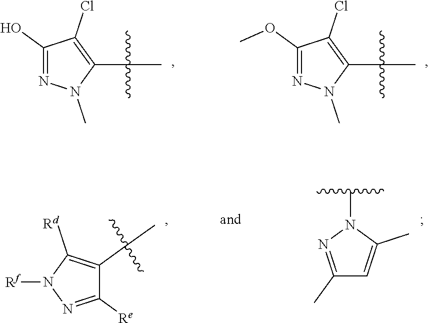

where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₆alkyl;
R^{e} is halo; C₁₋₄alkyl; C₁₋₄alkyl substituted with OH, OCH₃, SCH₃, or OCF₃; C₁₋₄haloalkyl; C₃₋₆cycloalkyl; or C₁₋₄haloalkyl substituted with OH, or OCH₃; and
R^{f} is H; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃;
or a pharmaceutically acceptable salt, solvate, stereoisomer, tautomer, isotopic variant, or N-oxide thereof.
27. The compound according to any of embodiments 1-19, wherein R³ is

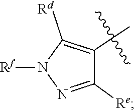

wherein
R^{d} is H, Cl, C₁₋₄alkyl or C₁₋₄haloalkyl;
R^{e} is halo, C₁₋₄alkyl, C₁₋₄haloalkyl or cyclopropyl; and
R^{f} is H;
or a pharmaceutically acceptable salt, solvate, stereoisomer, tautomer, isotopic variant, or N-oxide thereof.
28. The compound according to any of embodiments 1-19, wherein R³ is

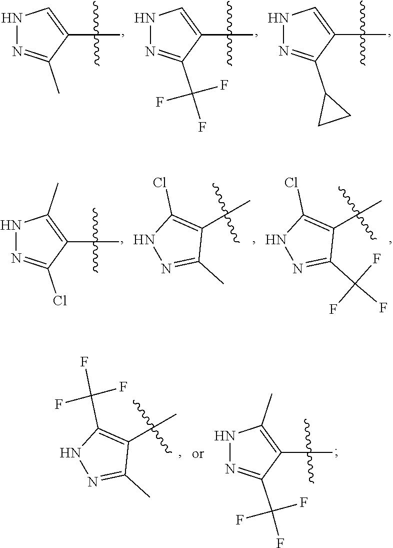

or a pharmaceutically acceptable salt, solvate, stereoisomer, tautomer, isotopic variant, or N-oxide thereof.
29. The compound according to any of embodiments 1-19, wherein R³ is

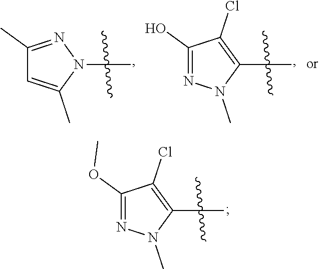

or a pharmaceutically acceptable salt, solvate, stereoisomer, tautomer, isotopic variant, or N-oxide thereof.
30. The compound according to embodiment 1, having the structure of Formula (IA):

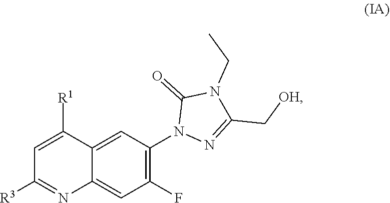

wherein
R¹ is selected from the group consisting of: C₁₋₆alkyl, C₁₋₄haloalkyl and

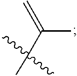

and
R³ is selected from the group consisting of:

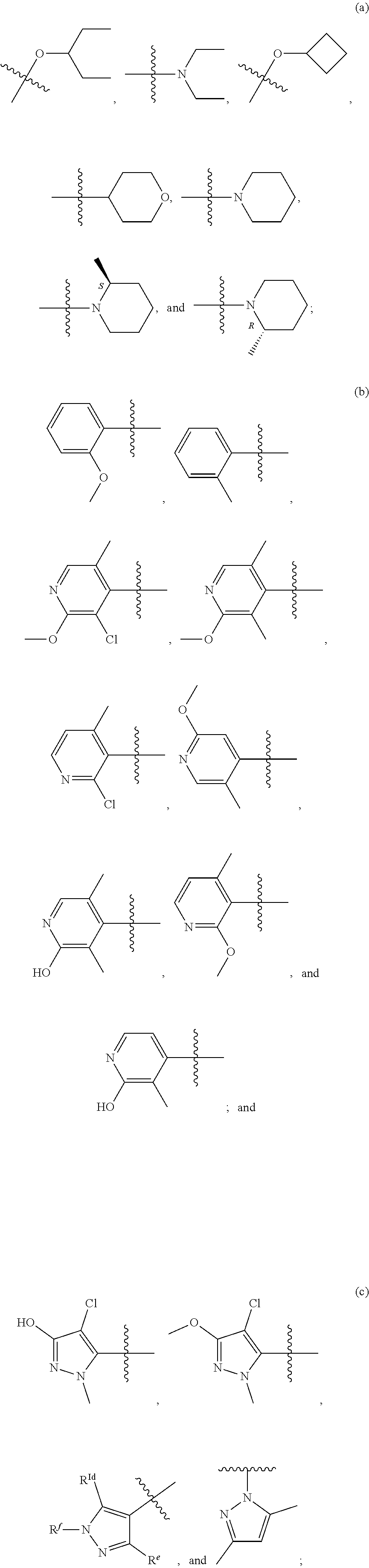

wherein
R^{d} is H, Cl, C₁₋₄alkyl or C₁₋₄haloalkyl;
R^{e} is halo, C₁₋₄alkyl, C₁₋₄haloalkyl or cyclopropyl; and
R^{f} is H;
   or a pharmaceutically acceptable salt, solvate, stereoisomer, tautomer, isotopic variant, or N-oxide thereof.
31. The compound according to embodiment 1, having the structure of Formula (IB):

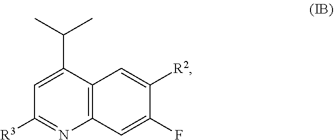

wherein
R² is

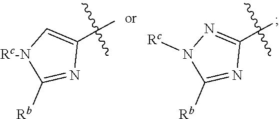

where
   R^{b} is C₁₋₄alkyl substituted with OH;
   and R^{c} is C₁₋₄alkyl;
R³ is

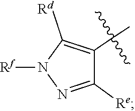

wherein
   R^{d} is H, Cl, C₁₋₄alkyl or C₁₋₄haloalkyl;
   R^{e} is halo, C₁₋₆alkyl, C₁₋₄haloalkyl or cyclopropyl; and
R^{f} is H;
or a pharmaceutically acceptable salt, solvate, stereoisomer, tautomer, isotopic variant, or N-oxide thereof.
32. The compound according to embodiment 1, having the structure of Formula (IC):

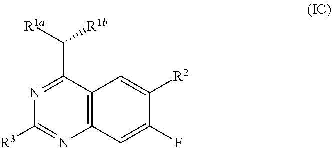

R^{1a} is CH₃;
R^{1b} is CH₃;
R² is

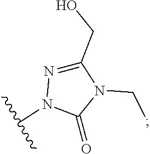

and
R³ is

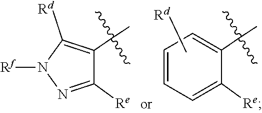

wherein
R^{d} is selected from the group consisting of: H, halo, C₁₋₆alkyl, and C₁₋₆haloalkyl;
R^{e} is selected from the group consisting of: halo, C₁₋₆alkyl, C₁₋₆haloalkyl, and cyclopropyl; and
R^{f} is H;
or a pharmaceutically acceptable salt, solvate, stereoisomer, tautomer, isotopic variant, or N-oxide thereof.
33. The compound according to embodiment 30, wherein R¹ is CH(CH₃)₂, CH(CH₃)(CF₃) or

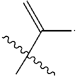

34. The compound according to embodiment 31, wherein R^{c} is CH₃.
35. The compound according to embodiment 32, wherein R^{e} is C₁₋₄alkyl.
36. A compound selected from the group consisting of:
4-Ethyl-1-(7-fluoro-4-isopropyl-2-(2-methoxyphenyl)quinolin-6-yl)-3-(hydroxymethyl)-1H-1,2,4-triazol-5 (4H)-one;
1-(2-(3-Chloro-5-methyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(5-methyl-3-(trifluoromethyl)-1H-pyrazol-4-yl) quinolin-6-yl)-3-(hydroxymethyl)-1H-1,2,4-triazol-5 (4H)-one;
4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one;
4-Ethyl-1-(7-fluoro-4-isopropyl-2-(pentan-3-yloxy)quinolin-6-yl)-3-(hydroxymethyl)-1H-1,2,4-triazol-5 (4H)-one;
1-(2-Cyclobutoxy-7-fluoro-4-isopropylquinolin-6-yl)-4-ethyl-3-(hydroxymethyl)-1H-1,2,4-triazol-5 (4H)-one;
1-(2-(3-Chloro-2-methoxy-5-methylpyridin-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(2-methoxy-5-methylpyridin-4-yl)quinolin-6-yl)-3-(hydroxymethyl)-1H-1,2,4-triazol-5 (4H)-one;
4-Ethyl-1-(7-fluoro-4-isopropyl-2-(o-tolyl)quinolin-6-yl)-3-(hydroxymethyl)-1H-1,2,4-triazol-5 (4H)-one;
4-Ethyl-2-(7-fluoro-4-isopropyl-2-(o-tolyl)quinazolin-6-yl)-5-(hydroxymethyl)-2,4-dihydro-3H-1,2,4-triazol-3-one;
4-Ethyl-2-(7-fluoro-4-isopropyl-2-(2-methoxy-4-methylpyridin-3-yl)quinolin-6-yl)-5-(hydroxymethyl)-2,4-dihydro-3H-1,2,4-triazol-3-one;
1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one;
1-(2-(Diethylamino)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(piperidin-1-yl)quinolin-6-yl)-3-(hydroxymethyl)-1H-1,2,4-triazol-5 (4H)-one;
1-(2-(3-Cyclopropyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-2-(7-fluoro-4-isopropyl-2-(3-methyl-1H-pyrazol-4-yl)quinolin-6-yl)-5-(hydroxymethyl)-2,4-dihydro-3H-1,2,4-triazol-3-one;
(S)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
(R)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
4-Ethyl-2-(7-fluoro-2-(2-hydroxy-3,5-dimethylpyridin-4-yl)-4-isopropylquinolin-6-yl)-5-(hydroxymethyl)-2,4-dihydro-3H-1,2,4-triazol-3-one;
4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-one;
6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide;
4-Ethyl-1-(7-fluoro-2-(2-hydroxy-3-methylpyridin-4-yl)-4-isopropylquinolin-6-yl)-3-(hydroxymethyl)-1H-1,2,4-triazol-5 (4H)-one;
4-Ethyl-1-(7-fluoro-4-isopropyl-2-(3-(trifluoromethyl)-1H-pyrazol-4-yl)quinolin-6-yl)-3-(hydroxymethyl)-1H-1,2,4-triazol-5 (4H)-one;
4-Ethyl-1-(7-fluoro-2-(2-hydroxy-5-methylpyridin-4-yl)-4-isopropylquinolin-6-yl)-3-(hydroxymethyl)-1H-1,2,4-triazol-5 (4H)-one;
2-(2-(4-Chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
2-(2-(4-Chloro-3-methoxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one;
(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol;
1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-ol;
(3-(2-(5-Chloro-3-methyl-1H-pyrazol-4-yl)-7-fluoro-4-isopropylquinolin-6-yl)-1-methyl-1H-1,2,4-triazol-5-yl)methanol;
1-(3-(2-(5-Chloro-3-methyl-1H-pyrazol-4-yl)-7-fluoro-4-isopropylquinolin-6-yl)-1-methyl-1H-1,2,4-triazol-5-yl)ethan-1-ol;
(4-(2-(5-Chloro-3-methyl-1H-pyrazol-4-yl)-7-fluoro-4-isopropylquinolin-6-yl)-1-methyl-1H-imidazol-2-yl)methanol;
(4-(7-Fluoro-4-isopropyl-2-(3-methyl-5-(trifluoromethyl)-1H-pyrazol-4-yl)quinolin-6-yl)-1-methyl-1H-imidazol-2-yl)methanol;
4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one;
1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one;
(S*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
(R*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
4-Ethyl-1-(7-fluoro-4-isopropyl-2-(o-methyl-d₃-phenyl)quinolin-6-yl)-3-(hydroxymethyl)-1H-1,2,4-triazol-5 (4H)-one; and
4-Ethyl-1-(7-fluoro-4-(prop-1-en-2-yl)-2-(o-tolyl)quinolin-6-yl)-3-(hydroxymethyl)-1H-1,2,4-triazol-5 (4H)-one;
or a pharmaceutically acceptable salt, solvate, stereoisomer, tautomer, isotopic variant, or N-oxide thereof.
37. A pharmaceutical composition comprising: (A) an effective amount of a compound according to any of embodiments 1-35, or a pharmaceutically acceptable salt, solvate, stereoisomer, tautomer, isotopic variant, or N-oxide thereof; and (B) at least one pharmaceutically acceptable excipient.
38. A pharmaceutical composition comprising an effective amount of a compound of embodiment 36, or a pharmaceutically acceptable salt, solvate, stereoisomer, tautomer, isotopic variant, or N-oxide thereof; and at least one pharmaceutically acceptable excipient.
39. A method of treating a subject suffering from or diagnosed with a disease, disorder, or medical condition comprising inhibiting or altering dihydroorotate oxygenase enzyme activity in the subject by administering to the subject an effective amount of at least one compound according to any of embodiments 1-35, or a pharmaceutically acceptable salt, solvate, stereoisomer, tautomer, isotopic variant, or N-oxide thereof.
40. The method according to embodiment 39, wherein the disorder, disease or medical condition is selected from the group consisting of: inflammatory disorders and autoimmune disorders.
41. The method according to embodiment 39, wherein the disorder, disease or medical condition is cancer.
42. The method according to embodiment 39, wherein the disorder, disease or medical condition is selected from the group consisting of: lymphomas, leukemias, carcinomas, and sarcomas.
43. The method according to embodiment 39, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.
44. The method according to embodiment 39, wherein the disorder, disease or medical condition is acute myeloid leukemia.
45. The method according to any of embodiments 39-44, wherein the at least one compound comprises a compound selected from the group consisting of:
4-Ethyl-1-(7-fluoro-4-isopropyl-2-(2-methoxyphenyl)quinolin-6-yl)-3-(hydroxymethyl)-1H-1,2,4-triazol-5 (4H)-one;
1-(2-(3-Chloro-5-methyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(5-methyl-3-(trifluoromethyl)-1H-pyrazol-4-yl) quinolin-6-yl)-3-(hydroxymethyl)-1H-1,2,4-triazol-5 (4H)-one;
4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one;
4-Ethyl-1-(7-fluoro-4-isopropyl-2-(pentan-3-yloxy)quinolin-6-yl)-3-(hydroxymethyl)-1H-1,2,4-triazol-5 (4H)-one;
1-(2-Cyclobutoxy-7-fluoro-4-isopropylquinolin-6-yl)-4-ethyl-3-(hydroxymethyl)-1H-1,2,4-triazol-5 (4H)-one;
1-(2-(3-Chloro-2-methoxy-5-methylpyridin-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(2-methoxy-5-methylpyridin-4-yl)quinolin-6-yl)-3-(hydroxymethyl)-1H-1,2,4-triazol-5 (4H)-one;
4-Ethyl-1-(7-fluoro-4-isopropyl-2-(o-tolyl)quinolin-6-yl)-3-(hydroxymethyl)-1H-1,2,4-triazol-5 (4H)-one;
4-Ethyl-2-(7-fluoro-4-isopropyl-2-(o-tolyl)quinazolin-6-yl)-5-(hydroxymethyl)-2,4-dihydro-3H-1,2,4-triazol-3-one;
4-Ethyl-2-(7-fluoro-4-isopropyl-2-(2-methoxy-4-methylpyridin-3-yl)quinolin-6-yl)-5-(hydroxymethyl)-2,4-dihydro-3H-1,2,4-triazol-3-one;
1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one;
1-(2-(Diethylamino)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(piperidin-1-yl)quinolin-6-yl)-3-(hydroxymethyl)-1H-1,2,4-triazol-5 (4H)-one;
1-(2-(3-Cyclopropyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-2-(7-fluoro-4-isopropyl-2-(3-methyl-1H-pyrazol-4-yl)quinolin-6-yl)-5-(hydroxymethyl)-2,4-dihydro-3H-1,2,4-triazol-3-one;
(S)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
(R)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
4-Ethyl-2-(7-fluoro-2-(2-hydroxy-3,5-dimethylpyridin-4-yl)-4-isopropylquinolin-6-yl)-5-(hydroxymethyl)-2,4-dihydro-3H-1,2,4-triazol-3-one;
4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-one;
6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide;
4-Ethyl-1-(7-fluoro-2-(2-hydroxy-3-methylpyridin-4-yl)-4-isopropylquinolin-6-yl)-3-(hydroxymethyl)-1H-1,2,4-triazol-5 (4H)-one;
4-Ethyl-1-(7-fluoro-4-isopropyl-2-(3-(trifluoromethyl)-1H-pyrazol-4-yl)quinolin-6-yl)-3-(hydroxymethyl)-1H-1,2,4-triazol-5 (4H)-one;
4-Ethyl-1-(7-fluoro-2-(2-hydroxy-5-methylpyridin-4-yl)-4-isopropylquinolin-6-yl)-3-(hydroxymethyl)-1H-1,2,4-triazol-5 (4H)-one;
2-(2-(4-Chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
2-(2-(4-Chloro-3-methoxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one;
(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol;
1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-ol;
(3-(2-(5-Chloro-3-methyl-1H-pyrazol-4-yl)-7-fluoro-4-isopropylquinolin-6-yl)-1-methyl-1H-1,2,4-triazol-5-yl)methanol;
1-(3-(2-(5-Chloro-3-methyl-1H-pyrazol-4-yl)-7-fluoro-4-isopropylquinolin-6-yl)-1-methyl-1H-1,2,4-triazol-5-yl)ethan-1-ol;
(4-(2-(5-Chloro-3-methyl-1H-pyrazol-4-yl)-7-fluoro-4-isopropylquinolin-6-yl)-1-methyl-1H-imidazol-2-yl)methanol;
(4-(7-Fluoro-4-isopropyl-2-(3-methyl-5-(trifluoromethyl)-1H-pyrazol-4-yl)quinolin-6-yl)-1-methyl-1H-imidazol-2-yl)methanol;
4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one;
1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one;
(S*)-4-Ethyl-1I-(7-fluoro-2-(o-tolyl)-4-(1,1,1-trifluoropropan-2-yl)quinolin-6-yl)-3-(hydroxymethyl)-1H-1,2,4-triazol-5 (4H)-one;
(R*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
4-Ethyl-1-(7-fluoro-4-isopropyl-2-(o-methyl-d₃-phenyl)quinolin-6-yl)-3-(hydroxymethyl)-1H-1,2,4-triazol-5 (4H)-one; and
4-Ethyl-1-(7-fluoro-4-(prop-1-en-2-yl)-2-(o-tolyl)quinolin-6-yl)-3-(hydroxymethyl)-1H-1,2,4-triazol-5 (4H)-one;
or a pharmaceutically acceptable salt, solvate, stereoisomer, tautomer, isotopic variant, or N-oxide thereof.

1. A compound having the structure of Formula (I): 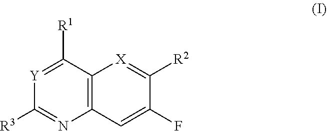
wherein
X is CH;
Y is CH or N;
R¹ is selected from the group consisting of: C₁₋₆alkyl; C₁₋₆alkyl substituted with OH, or OCH₃; C₂₋₆alkenyl; C₁₋₆haloalkyl; C₁₋₆haloalkyl substituted with OH, or OCH₃; C₃₋₆cycloalkyl; C₁₋₆cycloalkyl independently substituted with one, two, three or four members each independently selected from the group consisting of: halo, OH, C₁₋₆alkyl, and C₁₋₆haloalkyl; oxetanyl; tetrahydrofuranyl; and tetrahydropyranyl;
R² is selected from the group consisting of: 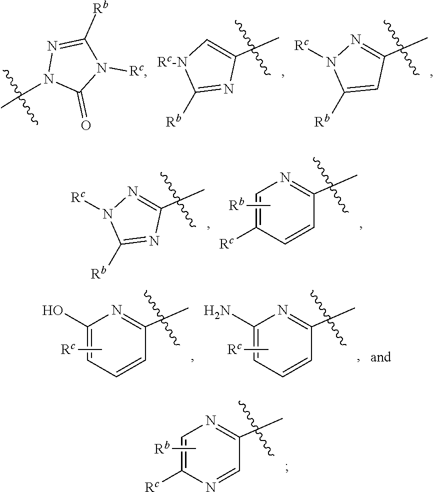
where
R^{b} is C₁₋₆alkyl substituted with a member selected from the group consisting of: OH, halo, CN, OC₁₋₆alkyl, OC₁₋₆haloalkyl and OC₃₋₆cycloalkyl;
R^{c} is selected from the group consisting of: C₁₋₆alkyl, C₁₋₆haloalkyl, C₃₋₆cycloalkyl, and tetrahydro-2H-pyranyl; and
R³ is selected from the group consisting of:
(a) O—(C₁₋₆alkyl), N(C₁₋₆alkyl)₂, piperidinyl, piperidinyl substituted with CH₃, O—C₃₋₆cycloalkyl, and N—C₃₋₆cycloalkyl; 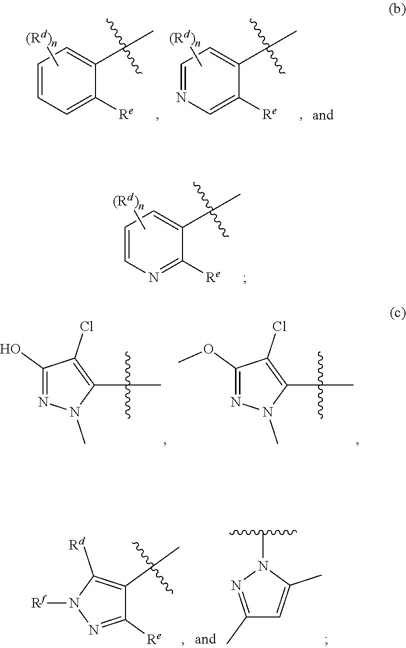
where
R^{d} is independently selected from the group consisting of: H; halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; N(CH₃)₂; OH; CN and OC₁₋₆alkyl;
R^{e} is selected from the group consisting of: halo; C₁₋₆alkyl; C₁₋₆alkyl substituted with a member selected from the group consisting of: OH, OCH₃, SCH₃, and OCF₃; C₁₋₆haloalkyl; C₁₋₆haloalkyl substituted with a member selected from the group consisting of: OH, and OCH₃; OH; OC₁₋₆alkyl; and C₁₋₆cycloalkyl;
R^{f} is selected from the group consisting of: H; C₁₋₆alkyl; C₁₋₆alkyl substituted with a member selected from the group consisting of: OH, OCH₃, SCH₃, and OCF₃; C₁₋₆haloalkyl; and C₁₋₆haloalkyl substituted with a member selected from the group consisting of: OH, and OCH₃; and
n is 1, or 2;
or a pharmaceutically acceptable salt, solvate, stereoisomer, tautomer, isotopic variant, or N-oxide thereof.

2. The compound according to claim 1, wherein Y is CH; or a pharmaceutically acceptable salt, solvate, stereoisomer, tautomer, isotopic variant, or N-oxide thereof.

3. The compound according to claim 1, wherein Y is N; or a pharmaceutically acceptable salt, solvate, stereoisomer, tautomer, isotopic variant, or N-oxide thereof.

4. The compound according to claim 1, wherein R¹ is C₁₋₄alkyl; C₁₋₄alkyl substituted with OH, or OCH₃; C₂₋₆alkenyl; C₁₋₄haloalkyl; C₁₋₄haloalkyl substituted with OH, or OCH₃; C₃₋₆cycloalkyl; C₃₋₆cycloalkyl independently substituted with one, two, three or four members each independently selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.

5. The compound according to claim 1, wherein R¹ is CH(CH₃)₂; or a pharmaceutically acceptable salt, solvate, stereoisomer, tautomer, isotopic variant, or N-oxide thereof.

6. The compound according to claim 1, wherein R¹ is CH(CH₃)(CF₃); or a pharmaceutically acceptable salt, solvate, stereoisomer, tautomer, isotopic variant, or N-oxide thereof.

7. The compound according to claim 1, wherein R¹ is 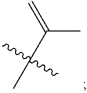 [SMILES: *C(=C)C]
or a pharmaceutically acceptable salt, solvate, stereoisomer, tautomer, isotopic variant, or N-oxide thereof.

8. The compound according to claim 1, wherein R¹ is cyclopropyl, cyclobutyl, cyclopentyl, or cyclohexyl; cyclopropyl, cyclobutyl, cyclopentyl, or cyclohexyl each independently substituted with one, two, three or four members selected from the group consisting of: halo, OH, C₁₋₄alkyl, and C₁₋₄haloalkyl; oxetanyl; tetrahydrofuranyl; and tetrahydropyranyl; or a pharmaceutically acceptable salt, solvate, stereoisomer, tautomer, isotopic variant, or N-oxide thereof.

9. The compound according to claim 1, wherein R² is 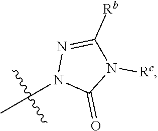
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

10. The compound according to claim 1, wherein R² is 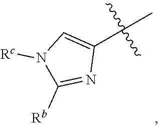
where
R^{b} is C₁₋₄alkyl substituted with OH; and R^{c} is C₁₋₄alkyl; or a pharmaceutically acceptable salt, solvate, stereoisomer, tautomer, isotopic variant, or N-oxide thereof.

11. The compound according to claim 1, wherein R² is 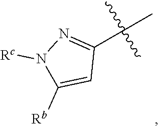
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

12. The compound according to claim 1, wherein R² is 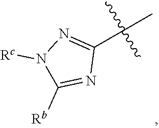
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

13. The compound according to claim 1, wherein R² is 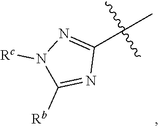
where
R^{b} is C₁₋₄alkyl substituted with OH; and R^{c} is C₁₋₄alkyl; or a pharmaceutically acceptable salt, solvate, stereoisomer, tautomer, isotopic variant, or N-oxide thereof.

14. The compound according to claim 1, wherein R² is 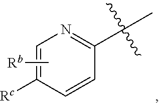
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

15. The compound according to claim 1, wherein R² is 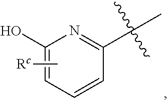
where
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

16. The compound according to claim 1, wherein R² is 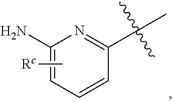
where
R^{c} is C₁₋₄alkyl, C₁₋₄haloalkyl, or C₁₋₆cycloalkyl; or a pharmaceutically acceptable salt, solvate, stereoisomer, tautomer, isotopic variant, or N-oxide thereof.

17. The compound according to claim 1, wherein R² is 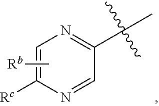
where
R^{b} is C₁₋₄alkyl substituted with OH, halo, CN, OC₁₋₄alkyl, OC₁₋₄haloalkyl or OC₃₋₆cycloalkyl; and
R^{c} is C₁₋₄alkyl, C₁₋₄haloalkyl, or C₃₋₆cycloalkyl; or a pharmaceutically acceptable salt, solvate, stereoisomer, tautomer, isotopic variant, or N-oxide thereof.

18. The compound according to claim 1,
wherein R² is 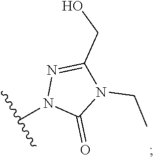 [SMILES: *n1nc(CO)n(CC)c1=O]
or a pharmaceutically acceptable salt, solvate, stereoisomer, tautomer, isotopic variant, or N-oxide thereof.

19. The compound according to claim 1, wherein R³ is O—(C₁₋₄alkyl), N(C₁₋₄alkyl)₂, piperidinyl, piperidinyl substituted with CH₃, O—C₃₋₆cycloalkyl, or N—C₃₋₆cycloalkyl; or a pharmaceutically acceptable salt, solvate, stereoisomer, tautomer, isotopic variant, or N-oxide thereof.

20. The compound according to claim 1, wherein R³ is 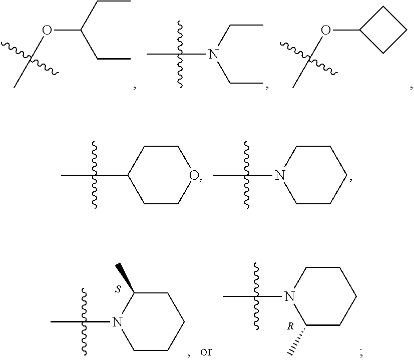
or a pharmaceutically acceptable salt, solvate, stereoisomer, tautomer, isotopic variant, or N-oxide thereof.

21. The compound according to claim 1, wherein R³ is 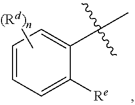
where
R^{d} is independently selected from the group consisting of: H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; CN; and OC₁₋₄alkyl;
R^{e} is halo; C₁₋₄alkyl; C₁₋₆alkyl substituted with OH, OCH₃, SCH₃, or OCF₃; C₁₋₄haloalkyl; or C₁₋₄haloalkyl substituted with OH, or OCH₃; and
n is 1 or 2;
or a pharmaceutically acceptable salt, solvate, stereoisomer, tautomer, isotopic variant, or N-oxide thereof.

22. The compound according to claim 1,
wherein R³ is 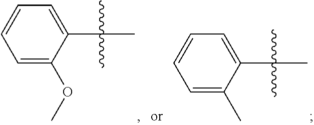
or a pharmaceutically acceptable salt, solvate, stereoisomer, tautomer, isotopic variant, or N-oxide thereof.

23. The compound according to claim 1, wherein R³ is 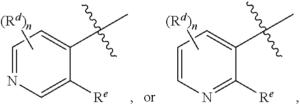
wherein
R^{d} is independently selected from the group consisting of: CH₃, OCH₃ and OH; R is halo, CH₃, or OCH₃; and n is 1 or 2;
or a pharmaceutically acceptable salt, solvate, stereoisomer, tautomer, isotopic variant, or N-oxide thereof.

24. The compound according to claim 1, wherein R³ is 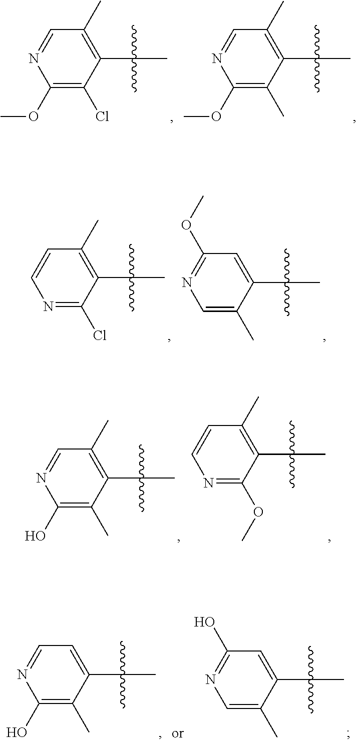
or a pharmaceutically acceptable salt, solvate, stereoisomer, tautomer, isotopic variant, or N-oxide thereof.

25. The compound according to claim 1, wherein R³ is selected from the group consisting of: 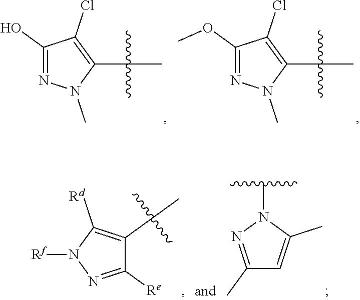
where
R^{d} is H; halo; C₁₋₄alkyl; C₁₋₄alkyl substituted with OH, OCH₃, SCH₃, or OCF₃; C₁₋₄haloalkyl; C₁₋₄haloalkyl substituted with OH, or OCH₃; or OC₁₋₄alkyl;
R^{e} is halo; C₁₋₄alkyl; C₁₋₄alkyl substituted with OH, OCH₃, SCH₃, or OCF₃; C₁₋₄haloalkyl; C₃₋₆cycloalkyl; or C₁₋₄haloalkyl substituted with OH, or OCH₃; and
R^{f} is H; C₁₋₄alkyl; C₁₋₄alkyl substituted with OH, OCH₃, SCH₃, or OCF₃; C₁₋₄haloalkyl; or C₁₋₄haloalkyl substituted with OH, or OCH₃;
or a pharmaceutically acceptable salt, solvate, stereoisomer, tautomer, isotopic variant, or N-oxide thereof.

26. The compound according to claim 1, wherein R³ is 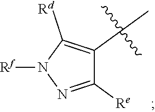
wherein
R^{d} is H, Cl, C₁₋₄alkyl or C₁₋₄haloalkyl;
R^{e} is halo, C₁₋₄alkyl, C₁₋₄haloalkyl or cyclopropyl; and
R_{f} is H;
or a pharmaceutically acceptable salt, solvate, stereoisomer, tautomer, isotopic variant, or N-oxide thereof.

27. The compound according to claim 1, wherein R³ is 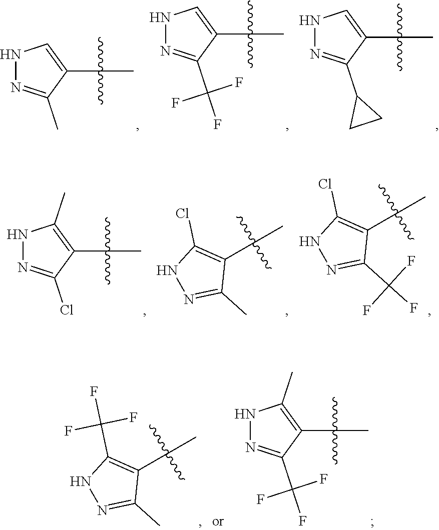
or a pharmaceutically acceptable salt, solvate, stereoisomer, tautomer, isotopic variant, or N-oxide thereof.

28. The compound according to claim 1, wherein R³ is 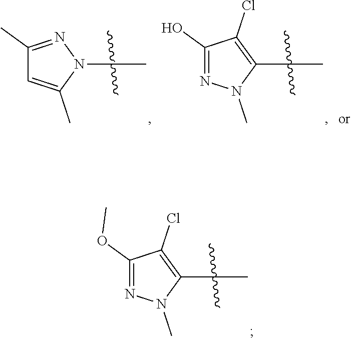
or a pharmaceutically acceptable salt, solvate, stereoisomer, tautomer, isotopic variant, or N-oxide thereof.

29. The compound according to claim 1, having the structure of Formula (IA): 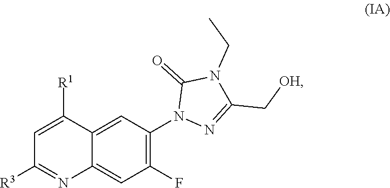
wherein
R¹ is selected from the group consisting of: C₁₋₆alkyl, C₁₋₆haloalkyl and 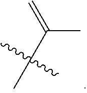 [SMILES: *C(=C)C]
and
R³ is selected from the group consisting of: 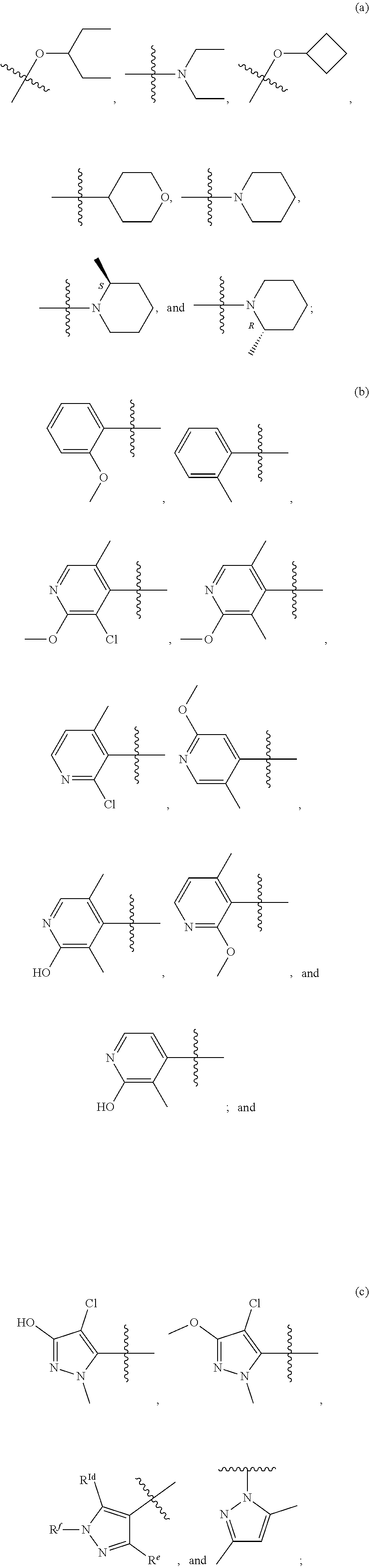
wherein
R^{d} is H, Cl, C₁₋₄alkyl or C₁₋₄haloalkyl;
R^{e} is halo, C₁₋₄alkyl, C₁₋₄haloalkyl or cyclopropyl; and
R^{f} is H;
or a pharmaceutically acceptable salt, solvate, stereoisomer, tautomer, isotopic variant, or N-oxide thereof.

30. The compound according to claim 1, having the structure of Formula (IB): 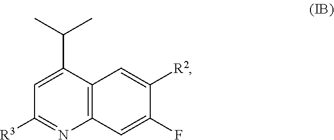
wherein 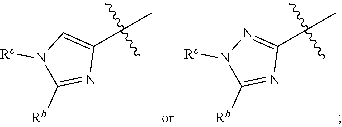
R² is
where
R^{b} is C₁₋₄alkyl substituted with OH;
and R^{c} is C₁₋₄alkyl;
R³ is 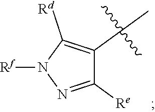
wherein
R^{d} is H, Cl, C₁₋₄alkyl or C₁₋₄haloalkyl;
R^{e} is halo, C₁₋₄alkyl, C₁₋₄haloalkyl or cyclopropyl; and
R^{f} is H;
or a pharmaceutically acceptable salt, solvate, stereoisomer, tautomer, isotopic variant, or N-oxide thereof.

31. The compound according to claim 1, having the structure of Formula (IC): 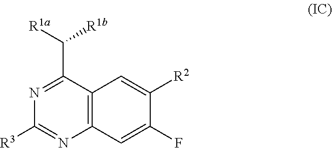
R^{1a} is CH₃;
R^{1b} is CH₃;
R² is 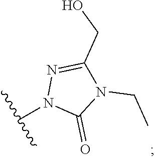 [SMILES: *n1nc(CO)n(CC)c1=O]
and
R³ is 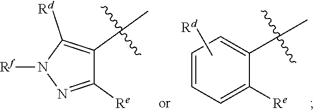
wherein
R^{d} is selected from the group consisting of: H, halo, C₁₋₆alkyl, and C₁₋₆haloalkyl;
R^{e} is selected from the group consisting of: halo, C₁₋₆alkyl, C₁₋₆haloalkyl, and cyclopropyl; and
R^{f} is H;
or a pharmaceutically acceptable salt, solvate, stereoisomer, tautomer, isotopic variant, or N-oxide thereof.

32. The compound according to claim 29, wherein R¹ is CH(CH₃)₂, CH(CH₃)(CF₃) or

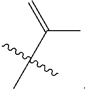 [SMILES: *C(=C)C]

33. The compound according to claim 30, wherein R^{c} is CH₃.

34. The compound according to claim 31, wherein R^{e} is C₁₋₄alkyl.

35. A compound selected from the group consisting of:
4-Ethyl-1-(7-fluoro-4-isopropyl-2-(2-methoxyphenyl)quinolin-6-yl)-3-(hydroxymethyl)-1H-1,2,4-triazol-5 (4H)-one;
1-(2-(3-Chloro-5-methyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(5-methyl-3-(trifluoromethyl)-1H-pyrazol-4-yl) quinolin-6-yl)-3-(hydroxymethyl)-1H-1,2,4-triazol-5 (4H)-one;
4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one;
4-Ethyl-1-(7-fluoro-4-isopropyl-2-(pentan-3-yloxy)quinolin-6-yl)-3-(hydroxymethyl)-1H-1,2,4-triazol-5 (4H)-one;
1-(2-Cyclobutoxy-7-fluoro-4-isopropylquinolin-6-yl)-4-ethyl-3-(hydroxymethyl)-1H-1,2,4-triazol-5 (4H)-one;
1-(2-(3-Chloro-2-methoxy-5-methylpyridin-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(2-methoxy-5-methylpyridin-4-yl)quinolin-6-yl)-3-(hydroxymethyl)-1H-1,2,4-triazol-5 (4H)-one;
4-Ethyl-1-(7-fluoro-4-isopropyl-2-(o-tolyl)quinolin-6-yl)-3-(hydroxymethyl)-1H-1,2,4-triazol-5 (4H)-one;
4-Ethyl-2-(7-fluoro-4-isopropyl-2-(o-tolyl)quinazolin-6-yl)-5-(hydroxymethyl)-2,4-dihydro-3H-1,2,4-triazol-3-one;
4-Ethyl-2-(7-fluoro-4-isopropyl-2-(2-methoxy-4-methylpyridin-3-yl)quinolin-6-yl)-5-(hydroxymethyl)-2,4-dihydro-3H-1,2,4-triazol-3-one;
1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one;
1-(2-(Diethylamino)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(piperidin-1-yl)quinolin-6-yl)-3-(hydroxymethyl)-1H-1,2,4-triazol-5 (4H)-one;
1-(2-(3-Cyclopropyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-2-(7-fluoro-4-isopropyl-2-(3-methyl-1H-pyrazol-4-yl)quinolin-6-yl)-5-(hydroxymethyl)-2,4-dihydro-3H-1,2,4-triazol-3-one;
(S)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
(R)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
4-Ethyl-2-(7-fluoro-2-(2-hydroxy-3,5-dimethylpyridin-4-yl)-4-isopropylquinolin-6-yl)-5-(hydroxymethyl)-2,4-dihydro-3H-1,2,4-triazol-3-one;
4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-one;
6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide;
4-Ethyl-1-(7-fluoro-2-(2-hydroxy-3-methylpyridin-4-yl)-4-isopropylquinolin-6-yl)-3-(hydroxymethyl)-1H-1,2,4-triazol-5 (4H)-one;
4-Ethyl-1-(7-fluoro-4-isopropyl-2-(3-(trifluoromethyl)-1H-pyrazol-4-yl)quinolin-6-yl)-3-(hydroxymethyl)-1H-1,2,4-triazol-5 (4H)-one;
4-Ethyl-1-(7-fluoro-2-(2-hydroxy-5-methylpyridin-4-yl)-4-isopropylquinolin-6-yl)-3-(hydroxymethyl)-1H-1,2,4-triazol-5 (4H)-one;
2-(2-(4-Chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
2-(2-(4-Chloro-3-methoxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one;
(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol;
1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-ol;
(3-(2-(5-Chloro-3-methyl-1H-pyrazol-4-yl)-7-fluoro-4-isopropylquinolin-6-yl)-1-methyl-1H-1,2,4-triazol-5-yl)methanol;
1-(3-(2-(5-Chloro-3-methyl-1H-pyrazol-4-yl)-7-fluoro-4-isopropylquinolin-6-yl)-1-methyl-1H-1,2,4-triazol-5-yl)ethan-1-ol;
(4-(2-(5-Chloro-3-methyl-1H-pyrazol-4-yl)-7-fluoro-4-isopropylquinolin-6-yl)-1-methyl-1H-imidazol-2-yl)methanol;
(4-(7-Fluoro-4-isopropyl-2-(3-methyl-5-(trifluoromethyl)-1H-pyrazol-4-yl)quinolin-6-yl)-1-methyl-1H-imidazol-2-yl)methanol;
4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one;
1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one;
(S*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
(R*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
4-Ethyl-1-(7-fluoro-4-isopropyl-2-(o-methyl-d₃-phenyl)quinolin-6-yl)-3-(hydroxymethyl)-1H-1,2,4-triazol-5 (4H)-one; and
4-Ethyl-1-(7-fluoro-4-(prop-1-en-2-yl)-2-(o-tolyl)quinolin-6-yl)-3-(hydroxymethyl)-1H-1,2,4-triazol-5 (4H)-one;
or a pharmaceutically acceptable salt, solvate, stereoisomer, tautomer, isotopic variant, or N-oxide thereof.

36. A pharmaceutical composition comprising: (A) an effective amount of a compound according to claim 1, or a pharmaceutically acceptable salt, solvate, stereoisomer, tautomer, isotopic variant, or N-oxide thereof; and (B) at least one pharmaceutically acceptable excipient.

37. A pharmaceutical composition comprising an effective amount of a compound of claim 35, or a pharmaceutically acceptable salt, solvate, stereoisomer, tautomer, isotopic variant, or N-oxide thereof; and at least one pharmaceutically acceptable excipient.

38. A method of treating a subject suffering from or diagnosed with a disease, disorder, or medical condition comprising inhibiting or altering dihydroorotate oxygenase enzyme activity in the subject by administering to the subject an effective amount of at least one compound according to claim 1, or a pharmaceutically acceptable salt, solvate, stereoisomer, tautomer, isotopic variant, or N-oxide thereof.

39. The method according to claim 38, wherein the disorder, disease or medical condition is selected from the group consisting of: inflammatory disorders and autoimmune disorders.

40. The method according to claim 38, wherein the disorder, disease or medical condition is cancer.

41. The method according to claim 38, wherein the disorder, disease or medical condition is selected from the group consisting of: lymphomas, leukemias, carcinomas, and sarcomas.

42. The method according to claim 38, wherein the disorder, disease or medical condition is selected from the group consisting of: acute lymphoblastic leukemia, acute myeloid leukemia, (acute) T-cell leukemia, acute lymphoblastic leukemia, acute lymphocytic leukemia, acute monocytic leukemia, acute promyelocytic leukemia, bisphenotypic B myelomonocytic leukemia, chronic lymphocytic leukemia, chronic myelogenous leukemia, chronic myeloid leukemia, chronic myelomonocytic leukemia, large granular lymphocytic leukemia, plasma cell leukemia, and also myelodysplastic syndrome, which can develop into an acute myeloid leukemia.

43. The method according to claim 38, wherein the disorder, disease or medical condition is acute myeloid leukemia.

44. The method according to any claim 38, wherein the at least one compound comprises a compound selected from the group consisting of:
4-Ethyl-1-(7-fluoro-4-isopropyl-2-(2-methoxyphenyl)quinolin-6-yl)-3-(hydroxymethyl)-1H-1,2,4-triazol-5 (4H)-one;
1-(2-(3-Chloro-5-methyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(5-methyl-3-(trifluoromethyl)-1H-pyrazol-4-yl) quinolin-6-yl)-3-(hydroxymethyl)-1H-1,2,4-triazol-5 (4H)-one;
4-Ethyl-1-(7-fluoro-4-isopropyl-2-(2-methoxy-3,5-dimethylpyridin-4-yl)quinolin-6-yl)-3-(hydroxymethyl)-1H-1,2,4-triazol-5 (4H)-one;
4-Ethyl-1-(7-fluoro-4-isopropyl-2-(pentan-3-yloxy)quinolin-6-yl)-3-(hydroxymethyl)-1H-1,2,4-triazol-5 (4H)-one;
1-(2-Cyclobutoxy-7-fluoro-4-isopropylquinolin-6-yl)-4-ethyl-3-(hydroxymethyl)-1H-1,2,4-triazol-5 (4H)-one;
1-(2-(3-Chloro-2-methoxy-5-methylpyridin-4-yl)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(2-methoxy-5-methylpyridin-4-yl)quinolin-6-yl)-3-(hydroxymethyl)-1H-1,2,4-triazol-5 (4H)-one;
4-Ethyl-1-(7-fluoro-4-isopropyl-2-(o-tolyl)quinolin-6-yl)-3-(hydroxymethyl)-1H-1,2,4-triazol-5 (4H)-one;
4-Ethyl-2-(7-fluoro-4-isopropyl-2-(o-tolyl)quinazolin-6-yl)-5-(hydroxymethyl)-2,4-dihydro-3H-1,2,4-triazol-3-one;
4-Ethyl-2-(7-fluoro-4-isopropyl-2-(2-methoxy-4-methylpyridin-3-yl)quinolin-6-yl)-5-(hydroxymethyl)-2,4-dihydro-3H-1,2,4-triazol-3-one;
1-(2-(3,5-Dimethyl-1H-pyrazol-1-yl)-7-fluoro-4-isopropylquinolin-6-yl)-4-ethyl-3-(hydroxymethyl)-1H-1,2,4-triazol-5 (4H)-one;
1-(2-(Diethylamino)-7-fluoro-4-isopropylquinolin-6-yl)-4-ethyl-3-(hydroxymethyl)-1H-1,2,4-triazol-5 (4H)-one;
4-Ethyl-1-(7-fluoro-4-isopropyl-2-(piperidin-1-yl)quinolin-6-yl)-3-(hydroxymethyl)-1H-1,2,4-triazol-5 (4H)-one;
1-(2-(3-Cyclopropyl-1H-pyrazol-4-yl)-7-fluoro-4-isopropylquinolin-6-yl)-4-ethyl-3-(hydroxymethyl)-1H-1,2,4-triazol-5 (4H)-one;
4-Ethyl-2-(7-fluoro-4-isopropyl-2-(3-methyl-1H-pyrazol-4-yl)quinolin-6-yl)-5-(hydroxymethyl)-2,4-dihydro-3H-1,2,4-triazol-3-one;
(S)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
(R)-4-Ethyl-2-(7-fluoro-4-isopropyl-2-(2-methylpiperidin-1-yl)quinolin-6-yl)-5-(hydroxymethyl)-2,4-dihydro-3H-1,2,4-triazol-3-one;
4-Ethyl-2-(7-fluoro-2-(2-hydroxy-3,5-dimethylpyridin-4-yl)-4-isopropylquinolin-6-yl)-5-(hydroxymethyl)-2,4-dihydro-3H-1,2,4-triazol-3-one;
4-Ethyl-1-(7-fluoro-4-isopropyl-2-(tetrahydro-2H-pyran-4-yl)quinolin-6-yl)-3-(hydroxymethyl)-1H-1,2,4-triazol-5 (4H)-one;
6-(4-Ethyl-3-(hydroxymethyl)-5-oxo-4,5-dihydro-1H-1,2,4-triazol-1-yl)-7-fluoro-4-isopropyl-2-(o-tolyl)quinoline 1-oxide;
4-Ethyl-1-(7-fluoro-2-(2-hydroxy-3-methylpyridin-4-yl)-4-isopropylquinolin-6-yl)-3-(hydroxymethyl)-1H-1,2,4-triazol-5 (4H)-one;
4-Ethyl-1-(7-fluoro-4-isopropyl-2-(3-(trifluoromethyl)-1H-pyrazol-4-yl)quinolin-6-yl)-3-(hydroxymethyl)-1H-1,2,4-triazol-5 (4H)-one;
4-Ethyl-1-(7-fluoro-2-(2-hydroxy-5-methylpyridin-4-yl)-4-isopropylquinolin-6-yl)-3-(hydroxymethyl)-1H-1,2,4-triazol-5 (4H)-one;
2-(2-(4-Chloro-3-hydroxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
2-(2-(4-Chloro-3-methoxy-1-methyl-1H-pyrazol-5-yl)-7-fluoro-4-isopropylquinolin-6-yl)-4-ethyl-5-(hydroxymethyl)-2,4-dihydro-3H-1,2,4-triazol-3-one;
1-(2-(5-Chloro-3-(trifluoromethyl)-1H-pyrazol-4-yl)-7-fluoro-4-isopropylquinolin-6-yl)-4-ethyl-3-(hydroxymethyl)-1H-1,2,4-triazol-5 (4H)-one;
(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)methanol;
1-(3-(7-Fluoro-4-isopropyl-2-(3-methyl-5-(trifluoromethyl)-1H-pyrazol-4-yl)quinolin-6-yl)-1-methyl-1H-1,2,4-triazol-5-yl)ethan-1-ol;
(3-(2-(5-Chloro-3-methyl-1H-pyrazol-4-yl)-7-fluoro-4-isopropylquinolin-6-yl)-1-methyl-1H-1,2,4-triazol-5-yl)methanol;
1-(3-(2-(5-Chloro-3-methyl-1H-pyrazol-4-yl)-7-fluoro-4-isopropylquinolin-6-yl)-1-methyl-1H-1,2,4-triazol-5-yl)ethan-1-ol;
(4-(2-(5-Chloro-3-methyl-1H-pyrazol-4-yl)-7-fluoro-4-isopropylquinolin-6-yl)-1-methyl-1H-imidazol-2-yl)methanol;
(4-(7-Fluoro-4-isopropyl-2-(3-methyl-5-(trifluoromethyl)-1H-pyrazol-4-yl)quinolin-6-yl)-1-methyl-1H-imidazol-2-yl)methanol;
4-Ethyl-2-(7-fluoro-4-isopropyl-2-(3-methyl-5-(trifluoromethyl)-1H-pyrazol-4-yl)quinazolin-6-yl)-5-(hydroxymethyl)-2,4-dihydro-3H-1,2,4-triazol-3-one;
1-(2-(2-Chloro-4-methylpyridin-3-yl)-7-fluoro-4-isopropylquinolin-6-yl)-4-ethyl-3-(hydroxymethyl)-1H-1,2,4-triazol-5 (4H)-one;
(S*)-4-Ethyl-)-(7-fluoro-2-(o-tolyl)-4-(1,1,1-trifluoropropan-2-yl)quinolin-6-yl)-3-(hydroxymethyl)-1H-1,2,4-triazol-5 (4H)-one;
(R*)-4-Ethyl-1-(7-fluoro-2-(o-tolyl)-4-(1,1,1-trifluoropropan-2-yl)quinolin-6-yl)-3-(hydroxymethyl)-1H-1,2,4-triazol-5 (4H)-one;
4-Ethyl-1-(7-fluoro-4-isopropyl-2-(o-methyl-d₃-phenyl)quinolin-6-yl)-3-(hydroxymethyl)-1H-1,2,4-triazol-5 (4H)-one; and
4-Ethyl-1-(7-fluoro-4-(prop-1-en-2-yl)-2-(o-tolyl)quinolin-6-yl)-3-(hydroxymethyl)-1H-1,2,4-triazol-5 (4H)-one;
or a pharmaceutically acceptable salt, solvate, stereoisomer, tautomer, isotopic variant, or N-oxide thereof.
